(19) United States
(12) Patent Application Publication
(10) Pub. No.: US 2025/0032614 A1
(43) Pub. Date: Jan. 30, 2025

(54) METHODS FOR TREATING STIFF PERSON SYNDROME AND MYASTHENIA GRAVIS USING ANTI-CD19 CAR-T CELL THERAPEUTICS

(71) Applicant: Kyverna Therapeutics, Inc., Emeryville, CA (US)

(72) Inventors: Dominique C. Borie, Palo Alto, CA (US); James Chung, Thousand Oaks, CA (US); Kunbin Qu, Emeryville, CA (US); Marshelle Warren Smith, Waltham, MA (US)

(21) Appl. No.: 18/827,646

(22) Filed: Sep. 6, 2024

Related U.S. Application Data
(63) Continuation of application No. PCT/US2024/039857, filed on Jul. 26, 2024.
(60) Provisional application No. 63/570,054, filed on Mar. 26, 2024.
(60) Provisional application No. 63/620,673, filed on Jan. 12, 2024.
(60) Provisional application No. 63/596,770, filed on Nov. 7, 2023.
(60) Provisional application No. 63/596,951, filed on Nov. 7, 2023.
(60) Provisional application No. 63/516,378, filed on Jul. 28, 2023.

(51) Int. Cl. A61K 39/00; A61P 21/00; A61P 21/04

(52) U.S. Cl. CPC A61K 39/4611; A61K 39/4631; A61K 39/464412; A61P 21/00; A61P 21/04; A61K 2239/21; A61K 2239/22; A61K 2239/31; A61K 2239/38

(57) ABSTRACT

Provided herein are methods and compositions for treating a subject having myasthenia gravis using T cells engineered with a chimeric antigen receptor that binds CD19. Also provided herein are methods and compositions for treating a subject having stiff person syndrome using T cells engineered with a chimeric antigen receptor that binds CD19.

### RELATED APPLICATIONS

This application is a continuation of International Application No. PCT/US2024/039857 filed on Jul. 26, 2024, which claims the benefit of and priority to U.S. Provisional Patent Application No. 63/516,378, filed on Jul. 28, 2023; U.S. Provisional Patent Application No. 63/596,770, filed on Nov. 7, 2023; U.S. Provisional Patent Application No. 63/596,951, filed on Nov. 7, 2023; U.S. Provisional Patent Application No. 63/620,673, filed on Jan. 12, 2024; and U.S. Provisional Patent Application No. 63/570,054, filed on Mar. 26, 2024, the entire contents of each of which are incorporated by reference herein for all purposes.

### SEQUENCE LISTING

This application contains a Sequence Listing which has been submitted electronically in XML format and is hereby incorporated by reference in its entirety. The XML copy, created on Jul. 26, 2024, is titled KVN-010USWOC1.xml and is 53,597 bytes in size.

### TECHNICAL FIELD

The present disclosure generally relates to T cells engineered to express chimeric antigen receptors (CARs) (e.g., fully human anti-CD19 CARs) and their use in the treatment and/or prevention of stiff person syndrome and myasthenia gravis.

### BACKGROUND

One of the most common autoimmune disorders affecting the neuromuscular junction is myasthenia gravis. Myasthenia gravis is a heterogeneous disease from a phenotypic and pathogenesis standpoint, associated with a spectrum of clinical symptoms ranging from purely ocular to severe weakness of limb, bulbar, and respiratory muscles. The weakness is due to an antibody-mediated, immunologic attack directed at proteins in the postsynaptic membrane of the neuromuscular junction (acetylcholine receptors or receptor-associated proteins), highlighting the central role of B lymphocytes in the pathogenesis. The age of onset is variable from childhood to late adulthood with disease peaks in younger adult women and older men.

Stiff person syndrome (SPS) is a rare disorder of the central nervous system characterized by rigidity and stimulus triggered painful muscle spasms of predominantly axial and proximal limb muscles. SPS has an insidious onset with gradual worsening over time and, if left untreated, can lead to permanent disability and mortality.

There is no cure for myasthenia gravis or SPS. Myasthenia gravis is generally treated with medications known as acetylcholinesterase inhibitors, such as neostigmine and pyridostigmine. Immunosuppressants, such as prednisone or azathioprine, may also be used, and in most cases must be continued long-term to maintain the disease. However, acetylcholinesterase inhibitors are toxic at high doses and only provide limited benefits in severely affected myasthenia gravis patients. Further, long-term immunosuppressant use increases the risks of infection, malignancy, cardiovascular disease, and bone marrow suppression, among other things. Similar issues are present in the treatment of SPS, as there is also no cure for this disease. SPS is generally treated with medications such as muscle relaxers, such as diazepam, clonazepam, baclofen, and tizanidine. Long term immunosuppressant usage is also common among SPS patients, leading to increased risks associated therewith. Thus, with regard to present treatment options for myasthenia gravis and SPS, there are limitations to their chronic use and of the use of multiple such agents in addition to their efficacy limitations. There remains a need for new therapies that overcome these limitations.

### SUMMARY

The present disclosure encompasses the recognition that myasthenia gravis is a chronic autoimmune disease mediated by autoantibodies affecting the neuromuscular junction and characterized by muscle weakness. The present disclosure appreciates that there are two clinical forms of myasthenia gravis: ocular myasthenia gravis and generalized myasthenia gravis. In ocular myasthenia gravis, weakness is limited to the eyelids and extraocular muscles whereas in generalized myasthenia gravis, weakness involves a combination of ocular, bulbar, limb, and respiratory muscles. The clinical manifestations of myasthenia gravis can vary from mild focal weakness in some patients to severe paresis, bulbar weakness and respiratory failure in others. Symptom severity can vary substantially in an individual over the course of a day and over time. Acute exacerbations of myasthenic symptoms can occur spontaneously or be precipitated by infection, surgery, pregnancy, childbirth, and certain medications and when severe, can lead to life-threatening neuromuscular respiratory failure known as myasthenic crisis needing intensive care treatment. The present disclosure further appreciates that a significant population of patients with myasthenia gravis are refractory to treatment, or limited in treatment options due to toxicities associated with conventional immunosuppressive and immunomodulatory therapies. For example, approximately 10 percent of patients with generalized myasthenia gravis have a treatment refractory form or are not suitable for treatment with standard of care options.

Provided herein are methods and compositions for treating a subject with myasthenia gravis (e.g., ocular myasthenia gravis or generalized myasthenia gravis). In some embodiments, the myasthenia gravis is Class I, Class II (e.g., Class IIa or IIb), Class III (e.g., Class IIIa or IIIb), Class IV (e.g., Class IVa or IVb), or Class V according to the Myasthenia Gravis Foundation of America (MGFA) clinical classification. In some embodiments, the myasthenia gravis is Class I. In some embodiments, the myasthenia gravis is Class II. In some embodiments, the myasthenia gravis is Class III. In some embodiments, the myasthenia gravis is Class IV. In some embodiments, the myasthenia gravis is Class IVa. In some embodiments, the myasthenia gravis is Class IVb. In some embodiments, the myasthenia gravis is Class V. Also provided herein are methods and compositions for treating a subject with stiff person syndrome (SPS) (e.g., treatment refractory SPS). Notably, provided herein are methods and compositions for treating a subject with myasthenia gravis or SPS for which conventional therapeutic regimens have been shown to be ineffective (e.g., due to their toxicity profiles or limitations in their pharmacological action). In some embodiments, conventional therapeutic regimens (i.e., standard of care (SOC) therapy) are withdrawn so that patients are free of any supportive immunomodulatory drugs for a duration of time (e.g., at least 1 week, 2 weeks, 3 weeks, 4 weeks, 1 month, 2 months, 3 months, 4 months, 6 months, 8 months, 1 year, 2 years, 3 years, or more) after receiving provided methods and compositions. In some embodiments, conventional therapeutic regimens (i.e., SOC therapy) are withdrawn ahead of collecting host cells (e.g., lymphocytes, such as T cells) to be engineered by any method described herein (e.g., at least 1 day, 2 days, 3 days, 4 days, 5 days, 6 days, 7 days, 8 days, 9 days, 10 day, 11 days, 12 days, 13 days, 14 days, 3 weeks, 4 weeks, or more before collecting host cells). Further provided herein are methods and compositions for treating a subject with myasthenia gravis or SPS, where said methods and compositions are used in place of known conventional therapeutic regimens. In some embodiments, the present disclosure provides for methods and compositions that result in elimination of B cells at tissue sites (e.g., in a subject with myasthenia gravis or SPS) normally not accessible to conventional treatments.

The methods of the present disclosure use T cells engineered to express anti-CD19 CAR constructs, which can reduce or deplete B cells responsible for one or more clinical symptoms of myasthenia gravis. In some embodiments, the methods of the present disclosure use T cells engineered to express anti-CD19 CAR constructs, which can reduce or deplete B cells responsible for one or more clinical symptoms of SPS. In many embodiments, the anti-CD19 CAR constructs have a lower toxicity profile as compared to conventional treatment. In some embodiments, an anti-CD19 CAR is substantially non-toxic to a subject receiving treatment with the CAR therapy. In some embodiments, such low toxicity or non-toxic CAR therapies provided by the present disclosure allow for higher doses and/or multiple doses which result in depletion of B cells at sites not treatable with conventional autoimmune treatments due to their toxicity profile. In some embodiments, a provided CAR therapy exhibits low risk of immunogenicity even when used in multiple dosing regimens at least in part due the CAR being a human construct, incorporating a fully human CD19-binding site. Indeed, CAR therapies provided herein remarkably exhibit low levels of toxicity commonly associated with CAR therapy (e.g., anti-CD19 CAR therapy), including cytokine-release syndrome (CRS) and neurologic toxicities.

In some embodiments, the present disclosure provides methods of and compositions for treating myasthenia gravis (e.g., Class I, Class II, Class III, Class IV, or Class V myasthenia gravis) with an anti-CD19 CAR. In some embodiments, the present disclosure provides methods of and compositions for treating SPS. In many embodiments of any of the provided methods and compositions described herein, an anti-CD19 CAR comprises a fully human chimeric antigen receptor comprising an extracellular antigen-binding domain, a transmembrane domain, and an intracellular domain, wherein the intracellular domain comprises a cytoplasmic signaling domain and one or more co-stimulatory domains. See, e.g., U.S. Pat. No. 10,287,350, which is incorporated by reference herein in its entirety.

The present disclosure provides, among other things, a method of treating stiff person syndrome (SPS), the method comprising administering to a subject in need thereof a therapeutically effective amount of T cells that comprises a vector comprising a nucleic acid encoding a chimeric antigen receptor (CAR), wherein the CAR comprises, from N-terminus to C-terminus: (a) an antigen-binding fragment of an anti-CD19 antibody; (b) a transmembrane domain; and (c) an intracellular T cell signaling domain from human CD3ζ.

In some embodiments, the subject has a high titer of anti-GAD65 antibodies in the serum (according to appropriate medical standard, e.g., at least 20 nM). In some embodiments, the subject is positive (e.g., tests positive) for anti-GAD65 antibodies in the cerebrospinal fluid (CSF). In some embodiments, the subject is positive (e.g., tests positive) for anti-glycine receptor (GlyR) antibodies in the serum. In some embodiments, the subject has a high titer of anti-GAD65 antibodies in the serum, is positive (e.g., tests positive) for anti-GAD65 antibodies in the CSF, and/or is positive (e.g., tests positive) for anti-GlyR antibodies in the serum. In some embodiments, the titer of anti-GAD65 antibodies prior to receiving a provided treatment is about 15 nmole/L, about 16 nmole/L, about 17 nmole/L, about 18 nmole/L, about 19 nmole/L, about 20 nmole/L, about 21 nmole/L, about 22 nmole/L, about 25 nmole/L, or greater. In some embodiments, the titer of anti-GAD65 antibodies is about 20 nmole/L.

In some embodiments, the subject has a high titer of anti-glycine receptor (anti-GlyR) antibodies in the serum, is positive (e.g., tests positive) for anti-GlyR antibodies in the CSF, and/or is positive (e.g., tests positive) anti-GlyR antibodies in the serum. In some embodiments, the subject has a high titer of anti-GABARAP antibodies in the serum, is positive (e.g., tests positive) for anti-GABARAP antibodies in the CSF, and/or is positive (e.g., tests positive) for anti-GABARAP antibodies in the serum. In some embodiments, the subject has a high titer of anti-amphiphysin antibodies in the serum, is positive (e.g., tests positive) for anti-amphiphysin antibodies in the CSF, and/or is positive (e.g., tests positive) for anti-amphiphysin antibodies in the serum.

In some embodiments, the subject has a stiffness index greater than or equal to 2.

In some embodiments, the subject has: (a) rigidity of limb and axial (trunk) muscles prominent in the abdominal and thoracolumbar paraspinal areas and making bending difficult; (b) continuous contraction of agonist and antagonist muscles; and/or (c) episodic spasms precipitated by unexpected noises, tactile stimuli, or emotional upset. In some embodiments, the subject has: (a) rigidity of limb and axial (trunk) muscles prominent in the abdominal and thoracolumbar paraspinal areas and making bending difficult; (b) continuous contraction of agonist and antagonist muscles; and (c) episodic spasms precipitated by unexpected noises, tactile stimuli, or emotional upset. In some embodiments, the subject has no other neurologic disease that could explain the stiffness and rigidity. In some embodiments, the subject has active symptoms (e.g., the symptoms of (a)-(c) above) with inadequate response to at least one immunomodulatory therapy selected from intravenous immunoglobulin (IVIG) therapy, rituximab, or plasmapheresis.

In some embodiments, the subject is ambulatory.

In some embodiments, the subject is positive (e.g., tests positive) for anti-amphiphysin antibodies. In some embodiments, the subject is negative (e.g., tests negative) for anti-amphiphysin antibodies. In some embodiments, the subject is positive (e.g., tests positive) for anti-GAD (e.g., anti-GAD65) antibodies. In some embodiments, the subject is negative (e.g., tests negative) for anti-GAD (e.g., anti-GAD65) antibodies. In some embodiments, the subject is positive (e.g., tests positive) for anti-GABARAP antibodies. In some embodiments, the subject is negative (e.g., tests negative) for anti-GABARAP antibodies. In some embodiments, the subject is positive (e.g., tests positive) for anti-GlyR antibodies. In some embodiments, the subject is negative (e.g., tests negative) for anti-GlyR antibodies.

The present disclosure provides, among other things, a method of treating myasthenia gravis, the method comprising administering to a subject in need thereof a therapeutically effective amount of T cells that comprises a vector comprising a nucleic acid encoding a chimeric antigen receptor (CAR), wherein the CAR comprises, from N-terminus to C-terminus: (a) an antigen-binding fragment of an anti-CD19 antibody; (b) a transmembrane domain; and (c) an intracellular T cell signaling domain from human CD3ζ.

In some embodiments, the myasthenia gravis is generalized myasthenia gravis (e.g., anti-AchR positive generalized myasthenia gravis). In some embodiments, the myasthenia gravis is of Class I, Class II (e.g., Class IIa or IIb), Class III (e.g., Class IIIa or IIIb), Class IV (e.g., Class IVa or IVb), or Class V according to the Myasthenia Gravis Foundation of America (MGFA) clinical classification. In some embodiments, the myasthenia gravis is of Class I according to the MGFA clinical classification. In some embodiments, the myasthenia gravis is of Class II according to the MGFA clinical classification. In some embodiments, the myasthenia gravis is of Class IIA according to the MGFA clinical classification. In some embodiments, the myasthenia gravis is of Class IIB according to the MGFA clinical classification. In some embodiments, the myasthenia gravis is of Class III according to the MGFA clinical classification. In some embodiments, the myasthenia gravis is of Class IV according to the MGFA clinical classification. In some embodiments, the myasthenia gravis is of Class IVa according to the MGFA clinical classification. In some embodiments, the myasthenia gravis is of Class IVb according to the MGFA clinical classification. In some embodiments, the myasthenia gravis is of Class V according to the MGFA clinical classification. In some embodiments, the subject is maintained at Class IIB disease by continuous treatment with intravenous immunoglobulin (IVIG) therapy or plasma exchange (PLEX).

In some embodiments, the subject has an MG-Activity of Daily Living (MG-ADL) total score of 6, 7, 8, 9, 10, 11, 12, 13, 14, 15, or greater. In some embodiments, the subject has an MG-ADL total score of greater than or equal to 6. In some embodiments, the subject has an MG-ADL total score of greater than or equal to 14. In some embodiments, the subject has an MG-ADL total score of greater than or equal to 15. In some embodiments, the subject has a score greater than or equal to 2 for at least one of the sub-components in the MG-ADL score, for talking, chewing, swallowing, or breathing.

In some embodiments, the subject has failed at least one monoclinal antibody treatment. For example, in some embodiments, the subject has failed, in the preceding 2 years, treatment with one or more (e.g., two or more) monoclonal antibodies with different mechanisms of action, or has failed at least one monoclonal antibody and requires chronic plasmapheresis or IVIG therapy to control symptoms. In some embodiments, the subject has failed two different monoclonal antibodies in the preceding 1 to 2 years. In some embodiments, the subject is on a stable dose of one or more glucocorticoids and/or other immunotherapies for at least 1 month or on a stable dose of azathioprine for at least 2 months. In some embodiments, the subject has received no change in dose of acetylcholinesterase inhibitors for at least 2 weeks. In some embodiments, the subject has not received IVIg or plasma exchange within the preceding 4 weeks.

In some embodiment, the subject has a forced vital capacity of about 3 liters or less, about 2.9 liters or less, about 2.8 liters or less, about 2.7 liters or less, about 2.6 liters or less, about 2.5 liters or less, about 2.4 liters or less, about 2.3 liters or less, about 2.2 liters or less, about 2.1 liters or less, about 2 liters or less, about 1.9 liters or less, about 1.8 liters or less, about 1.7 liters or less, about 1.6 liters or less, about 1.5 liters or less, about 1.4 liters or less, about 1.3 liters or less, about 1.2 liters or less, about 1.1 liters or less, or about 1 liter or less.

In some embodiments, the subject is positive (e.g., tests positive) for an autoantibody that binds acetylcholine receptor (AChR), muscle-specific kinase (MuSK), or lipoprotein-related protein 4 (LRP4). In some embodiments, the subject tests positive for anti-acetylcholine receptor (AChR) antibodies. In some embodiments, the subject tests negative for anti-AChR antibodies. In some embodiments, the subject tests positive for anti-muscle-specific kinase (MuSK) antibodies. In some embodiments, the subject tests negative for anti-MuSK antibodies. In some embodiments, the subject tests positive for anti-RyR antibodies. In some embodiments, the subject tests negative for anti-RyR antibodies. In some embodiments, the subject tests positive for titin antibodies. In some embodiments, the subject tests negative for titin antibodies. In some embodiments, the subject tests positive for anti-striated muscle antibodies. In some embodiments, the subject tests negative for anti-striated muscle antibodies. In some embodiments, the subject tests positive for anti-lipoprotein-related protein 4 (LRP4) antibodies. In some embodiments, the subject tests negative for anti-LRP4 antibodies. In some embodiments, the subject tests positive for IgG1 autoantibodies (e.g., IgG1 autoantibodies that bind any one of the autoantigens above). In some embodiments, the subject tests negative for IgG1 autoantibodies. In some embodiments, the subject tests positive for anti-voltage-gated calcium channel (VGCC, N type) antibodies. In some embodiments, the subject tests negative for anti-VGCC N type antibodies. In some embodiments, the subject tests positive for anti-calcium channel antibodies. In some embodiments, the subject tests negative for anti-calcium channel antibodies. In some embodiments, the subject tests negative for anti-AChR antibodies, but tests positive for one or more other myasthenia gravis related antibodies (e.g., anti-RyR antibodies, anti-titin antibodies, anti-striated muscle antibodies, anti-LRP4 antibodies, anti-VGCC N type antibodies, etc.). In some embodiments, the subject is seronegative for antibodies known to be associated with myasthenia gravis. In some embodiments, a subject with myasthenia gravis has also been identified as having one or more other co-existing autoimmune diseases (e.g., the subject has been diagnosed with concomitant myasthenia gravis and one or more other autoimmune diseases). In some embodiments, a subject with myasthenia gravis has also been identified as having co-existing Lambert-Eaton myasthenic syndrome (LEMS) (e.g., the subject has been diagnosed with concomitant myasthenia gravis and LEMS). In some embodiments, a subject with myasthenia gravis has also been identified as having co-existing Stiff Person Syndrome (SPS) (e.g., the subject has been diagnosed with concomitant myasthenia gravis and SPS). In some embodiments, a subject with myasthenia gravis has also been identified as having co-existing rheumatoid arthritis (e.g., the subject has been diagnosed with concomitant myasthenia gravis and rheumatoid arthritis).

In some embodiments of any of the foregoing aspects, directed to methods of treating myasthenia gravis or SPS, an anti-CD19 antibody is a human antibody.

In some embodiments, an antigen-binding fragment of the anti-CD19 antibody comprises a heavy chain variable domain and a light chain variable domain, the heavy chain variable domain comprising CDR1, CDR2, and CDR3 amino acid sequences of SEQ ID NOs: 1, 2, and 3, respectively, and the light chain variable domain comprising CDR1, CDR2, and CDR3 amino acid sequences of SEQ ID NOs: 4, 5, and 6, respectively.

In some embodiments, an antigen-binding fragment of the anti-CD19 antibody comprises a heavy chain variable domain and a light chain variable domain, the heavy chain variable domain comprising CDR1, CDR2, and CDR3 amino acid sequences of SEQ ID NOs: 25, 26, and 3, respectively, and the light chain variable domain comprising CDR1, CDR2, and CDR3 amino acid sequences of SEQ ID NOs: 4, 5, and 6, respectively.

In some embodiments, a heavy chain variable domain comprises an amino acid sequence at least 90% identical to SEQ ID NO: 7, and the light chain variable domain comprises an amino acid sequence at least 90% identical to SEQ ID NO: 8. In some embodiments, a heavy chain variable domain comprises the amino acid sequence of SEQ ID NO: 7, and the light chain variable domain comprises the amino acid sequence of SEQ ID NO: 8.

In some embodiments, an antigen-binding fragment of the anti-CD19 antibody comprises the amino acid sequence of SEQ ID NO: 17.

In some embodiments, a transmembrane domain is from human CD8.

In some embodiments, a transmembrane domain comprises an amino acid sequence at least 90% identical to SEQ ID NO: 11. In some embodiments, a transmembrane domain comprises the amino acid sequence of SEQ ID NO: 11.

In some embodiments, an intracellular T cell signaling domain from human CD3ζ comprises an amino acid sequence at least 90% identical to SEQ ID NO: 23. In some embodiments, an intracellular T cell signaling domain from human CD3ζ comprises the amino acid sequence of SEQ ID NO: 23.

In some embodiments, a CAR further comprises an intracellular T cell signaling domain from human CD28. In some embodiments, an intracellular T cell signaling domain from human CD28 comprises the amino acid sequence of SEQ ID NO: 21.

In some embodiments, a CAR does not comprise an intracellular T cell signaling domain from 4-1BB.

In some embodiments, a CAR comprises an amino acid sequence of SEQ ID NO: 10 or 13.

In some embodiments, a vector is a lentivirus vector. In some embodiments, a vector further comprises a murine stem cell virus (MSCV) U3 promoter operably linked to the nucleic acid.

In some embodiments, at least 10% (e.g., at least 15%, at least 20%, at least 25%, at least 30%, at least 40%, at least 50%, at least 60%, at least 70%, at least 80%, at least 90%, at least 95%, or at least 99%) of the provided T cells express a CAR (e.g., any CAR provided herein). In some embodiments, provided T cells comprise at least 10% (e.g., at least 15%, at least 20%, at least 25%, at least 30%, at least 40%) of CD8+ cytotoxic T cells. In some embodiments, provided T cells comprise at least 10% (e.g., at least 15%, at least 20%, at least 25%, at least 30%, at least 40%, at least 50%) of CD4+ helper T cells. In some embodiments, the provided CAR+ T cells comprise at least 10% (e.g., at least 15%, at least 20%, at least 25%, at least 30%, at least 40%) of CD8+ cytotoxic T cells and/or at least 10% (e.g., at least 15%, at least 20%, at least 25%, at least 30%, at least 40%, at least 50%) of CD4+ helper T cells.

In some embodiments, a therapeutically effective dose is in a range of about 5×10⁷ to 1×10⁸, about 5×10⁷ to 9×10⁷, about 5×10⁷ to 8×10⁷, about 5×10⁷ to 7×10⁷, about 5×10⁷ to 6×10⁷, about 6×10⁷ to 1×10⁸, about 7×10⁷ to 1×10⁸, about 8×10⁷ to 1×10⁸, about 9×10⁷ to 1×10⁸, about 6×10⁷ to 9×10⁷, or about 7×10⁷ to 8×10⁷ of the T cells. In some embodiments, a therapeutically effective dose is in a range of about 5×10⁷ to 1×10⁸ of the T cells. In some embodiments, a therapeutically effective dose is about 5×10⁷ of the T cells. In some embodiments, a therapeutically effective dose is about 1×10⁸ of the T cells.

In some embodiments, provided T cells are administered by intravenous infusion.

In some embodiments, the subject receives a single dose of the T cells.

In some embodiments, the subject has received a lymphodepletion treatment. In some embodiments, lymphodepletion treatment comprises intravenous administration of cyclophosphamide (e.g., at a dose of 300 mg/m²) and of fludarabine (e.g., at a dose of 30 mg/m²) prior to administration of the T cells, e.g., once every day for 3 days, starting 5 to 7 days prior to administration of the T cells. In some embodiments, lymphodepletion treatment comprises intravenous administration of cyclophosphamide (e.g., at a dose of 300 mg/m²) and of fludarabine (e.g., at a dose of 30 mg/m²) prior to administration of the T cells, e.g., once every day for 3 days, starting 6 days prior to administration of the T cells. In some embodiments, lymphodepletion treatment comprises intravenous administration of cyclophosphamide (e.g., at a dose of 300 mg/m²) and of fludarabine (e.g., at a dose of 30 mg/m²) prior to administration of the T cells, e.g., once every day for 3 days, starting 5 days prior to administration of the T cells. In some embodiments, the subject does not receive a lymphodepletion treatment prior to administration of the T cells. In some embodiments, the subject has received a minimized lymphodepletion treatment. In some embodiments, a minimized lymphodepletion treatment comprises intravenous administration of cyclophosphamide (e.g., at a dose of 150 mg/m²) and of fludarabine (e.g., at a dose of 15 mg/m²) prior to administration of the T cells, e.g., once every day for 3 days, starting 5 to 7 days prior to administration of the T cells. In some embodiments, a minimized lymphodepletion treatment reduces lymphocytes in a subject by about 30%, 35%, 40%, 45%, 50%, 55%, 60%, 65%, or 70% relative to the subject before receiving lymphodepletion treatment or another suitable control. In some embodiments, the subject has received a minimized lymphodepletion treatment resulting in about 50% reduction of lymphocytes in the subject relative to the subject before receiving lymphodepletion treatment or another suitable control. In some embodiments, the subject has not received a lymphodepletion treatment.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

Unless otherwise defined, all technical and scientific terms used herein have the same meaning as commonly understood by one of ordinary skill in the art to which this disclosure pertains. Although methods and materials similar or equivalent to those described herein can be used in accordance with the present disclosure, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1 is a schematic of a lentivirus vector encoding an anti-CD19 CAR transgene.

FIG. 2 is a series of graphs showing representative flow cytometric analyses of the frequency of circulating anti-CD19 CAR T cells among total CD3+ T cells in a subject with myasthenia gravis (see Example 2) on different days after infusion of 1×10⁸ KYV-101 CAR T cells along with the CD4+/CD8+ composition of CAR+ and CAR− T cells.

FIG. 3 is a series of graphs showing various measurements taken before and after infusion of an anti-CD19 CAR T cell in a subject with myasthenia gravis (see Example 2). The left panel displays the B-cell count (in cells/μL) before and after infusion (infusion=day 0) of anti-CD19 CAR T cells. The middle panel shows concentration of the pathogenic autoantibodies against the acetylcholine receptor (anti-AchR antibodies) immediately before infusion of anti-CD19 CAR T cells and at a follow-up day (day 62). The right panel shows the concentration of IgG titres, all of which are within the protective range, for tetanus (cIU/mL), varicella zoster virus (VZV, cIE/mL), rubella (IU/mL), mumps (AU/mL), and measles (AU/mL) before (day −7) and after (day 48) treatment with CAR T cells.

FIG. 4 is a series of graphs showing a summary of the clinical course of a subject treated with a provided anti-CD19 CAR T cell therapy (see Example 2), including arm holding test (top panel), walking distance (with/circle or without/square walker) (middle panel), and the multiparametric Besinger disease activity (left y-axis, circles) and the quantitative QMG myasthenia gravis score (right y-axis, squares) (bottom panel). Myasthenic crises requiring intubation and mechanical ventilation are highlighted by lightning bolt markers.

FIG. 5 is a series of graphs showing treatment history of a subject (see Example 2) up until infusion of CD19-CAR T-cells (top panel), along with the course of anti-AChR antibody titers (middle panel) and the subject's Besinger score (bottom panel). Fields of active medication in the top panel are depicted as shaded in relative to the background grid (e.g., prednisone, PYD retard, and PYD are active throughout the duration covered in the grid, whereas MMF was no longer active at day −7, and FluCy was no longer active at day 0). Abbreviations: MMF, mycophenolate mofetil; LD, lymphodepletion; FluCy, fludarabine and cyclophosphamide; PYD, pyridostigmine).

FIG. 6 Peripheral blood frequency of anti-CD19 CAR T cells (among total CD3+ T cells) (top panel) and their concentration (in cells/l) (middle panel) was determined upon CAR T-cell infusion (at day 0) by flow cytometry. Concentration of CD4+ and CD8+ CAR T cells was determined (bottom panel) in the infused product and the patient's peripheral blood.

FIG. 7 is a series of graphs showing various measurements related to tolerability of anti-CD19 CAR T cell infusion. (A) Average daily body temperature during the patient's hospitalization phase as measured following anti-CD19 CAR T-cell infusion. (B-D) Leukocyte count, platelet count, and hemoglobin (Hb) level were routinely determined. (E-F) Total IgG and IgM serum concentrations. (G-H) Serum levels of alanineaminotransferase (ALAT), and of aspartate-aminotransferase (ASAT) transaminases. The shaded area indicates the reference range.

FIGS. 8A-8D are a series of graphs showing functional testing of disease activity. FIG. 8A shows Besinger-Toyka score calculated throughout the treatment course by testing all eight possible items and dividing by eight. FIG. 8B shows quantitative score for myasthenia gravis (QMG) during treatment. All thirteen items were tested and divided by thirteen. FIG. 8C shows vital capacity in liters during treatment. FIG. 8D shows Myasthenia Gravis Activities of Daily Living (MG-ADL) evaluated throughout treatment. All eight items were assessed to ensure comprehensive analysis.

FIG. 9A-9D are a series of graphs showing laboratory analyses of T and B lymphocytes and antibody levels. FIG. 9A shows flow cytometric analyses of the counts of circulating anti-CD19 CAR-T cells at different days after infusion of 1×10⁸ KYV-101 CAR-T cells. FIG. 9B shows flow cytometric analyses of the counts of circulating CD19 B cells at different days after infusion of 1×10⁸ KYV-101 CAR-T cells. FIG. 9C shows serum concentrations of the pathogenic autoantibodies against the acetylcholine receptor (anti-AChR antibodies) before and after treatment with anti-CD19 CAR-T cells (dashed line: reference detection limit). FIG. 9D shows serum concentrations of the pathogenic autoantibodies against the voltage gated calcium channel (VGCC) N-type before and after treatment with anti-CD19 CAR-T cells (dashed line: reference detection limit).

FIG. 10 is a graph showing the reduction in QMG score observed in two myasthenia gravis patients treated with a provided CAR-T therapy. Patient #1 is described in Example 2, and Patient #2 is described in Example 3.

FIG. 11 is a timeline showing the course of treatment of a SPS patient prior to receiving KYV-101 CAR-T cell therapy.

FIG. 12 is a series of graphs showing (A) CAR T cell counts, (B) B cell counts, (C) Percentage of CD4 positive and CD8 positive T cells in the CD3 positive T cell population, and (D) the ratio of CD4 positive T cells to CD8 positive T cells in samples of a SPS patient treated with KYV-101 CAR-T cell therapy.

FIG. 13 is a graph showing anti-GAD antibody titer in a SPS patient before and after receiving KYV-101 CAR-T cell therapy.

FIG. 14 is a graph showing changes in stiffness in a SPS patient as judged by modified Ashworth scale (MAS) before and after receiving KYV-101 CAR-T cell therapy.

FIG. 15 is a graph showing changes in diazepam regimens in a SPS patient before and after receiving KYV-101 CAR-T cell therapy.

FIG. 16 is a graph showing changes in walking speed in a SPS patient treated with KYV-101 CAR-T cell therapy.

FIG. 17 is a graph showing changes in uninterrupted walking distance in a SPS patient before and after treatment with KYV-101 CAR-T cell therapy.

### DETAILED DESCRIPTION

### Definitions

“About” a number, as used herein, refers to range including the number and ranging from 10% below that number to 10% above that number. “About” a range refers to 10% below the lower limit of the range, spanning to 10% above the upper limit of the range.

As used herein, the term “antibody” refers to any immunoglobulin, whether naturally occurring or wholly or partially synthetically produced. All derivatives thereof which maintain specific binding ability are also included in the term. In some embodiments, the term “antibody” refers to any protein having a binding domain which is homologous or largely homologous to an immunoglobulin-binding domain. Antibody proteins may be derived from natural sources, or partly or wholly synthetically produced. An antibody may be monoclonal or polyclonal. An antibody may be a member of any immunoglobulin class, including any of the human classes: IgG, IgM, IgA, IgD, and IgE. In certain embodiments, an antibody may be a member of the IgG immunoglobulin class.

As used herein, “derived from” or “derivative” indicates a structural similarity and a functional similarity between a subject molecule and a reference molecule (e.g., between polynucleotides, polypeptides, etc.). With respect to structural similarity, the subject molecule does not necessarily comprise the same sequence (e.g., nucleic acid sequence, amino acid sequence, etc.) as the reference molecule, but has at least 75%, 80%, 85%, 90%, 91%, 92%, 93%, 94%, 95%, 96%, 97%, 98%, 99% or higher identity to the sequence (e.g., nucleic acid sequence, amino acid sequence, etc.) of the reference molecule or a fragment thereof, the fragment comprising at least 10%, 20%, 30%, 40%, 50%, 60%, 70%, 80%, 90%, 91%, 92%, 93%, 94%, 95%, 96%, 97%, 98%, 99%, or more of the sequence of the reference molecule. With respect to functional similarity, the subject molecule has at least 1%, 5%, 10%, 20%, 30%, 40%, 50%, 60%, 70%, 80%, 90%, 95%, 96%, 97%, 98%, 99% or more of an activity of the reference molecule or the fragment thereof as determined in a suitable assay. For example, a subject polypeptide may be considered to be derived from a reference polypeptide when the subject polypeptide has structural similarity, as defined above, to the reference polypeptide and retains certain function(s), such as certain intermolecular or intramolecular interactions (e.g., binding to a protein, e.g., a particular receptor, or a signaling activity), though such interactions could be stronger, equivalent, or weaker than that of the reference polypeptide. As another non-limiting example, a subject polynucleotide may be considered to be derived from a reference polynucleotide when the subject polynucleotide has structural similarity to the reference polynucleotide, as defined above, and encodes a protein or protein fragment that is a derivative of the protein encoded by the reference polynucleotide, or has the same or similar function (e.g., as a regulatory element, e.g., promoter or enhancer) as the reference polynucleotide. Functional similarity takes into account the context of the disclosure. For example, when applied to a subject intracellular T cell signaling domain derived from a reference protein (e.g., CD3ζ, CD28), the subject intracellular T cell signaling domain has structural and functional similarities to an intracellular T cell signaling domain of the reference protein as known in the art. Similarly, when applied to a subject transmembrane domains derived from a reference protein, the subject transmembrane domain has structural and functional similarities to a transmembrane domain of the reference protein as known in the art. In one non-limiting example, an intracellular T cell signaling domain derived from a CD3ζ molecule retains sufficient CD3ζ structure such that it has the ability to transduce a signal (e.g., ZAP-70 activation) under appropriate conditions.

As used herein, the term “functional fragment” of a reference biomolecule, e.g., a polynucleotide or polypeptide, refers to a shorter and/or smaller derivative of the reference biomolecule that has at least 75%, 80%, 85%, 90%, 91%, 92%, 93%, 94%, 95%, 96%, 97%, 98%, 99% or more sequence identity to the sequence of a fragment of the reference biomolecule.

As used herein the term “operably linked” refers to polynucleotide sequences placed into a functional relationship with one another. For instance, a promoter or enhancer is operably linked to a coding sequence if it regulates, or contributes to modulation of, the transcription of a coding sequence. Operably linked DNA sequences encoding regulatory sequences are typically contiguous to a coding sequence. However, enhancers can function when separated from a promoter by up to several kilobases or more. Additionally, multi-cistronic constructs can include multiple coding sequences which use only one promoter by including a 2A self-cleaving peptide, an IRES element, etc. Accordingly, some polynucleotide elements may be operably linked but not contiguous.

As used herein, the term “patient” or “subject” are used interchangeably to refer to any organism to which a compositions disclosed herein may be administered, e.g., for experimental, diagnostic, prophylactic, and/or therapeutic purposes. Typical patients include animals (e.g., mammals such as mice, rats, rabbits, non-human primates, and/or humans). In some embodiments, a patient or subject is a human. In some embodiments, a patient or subject is an adult human (e.g., at least 18 years of age). In some embodiments, a patient or subject is 18 to 75 years of age, inclusive. In many embodiments, a patient is a human subject suffering from myasthenia gravis.

### Methods and Compositions for Treating Myasthenia Gravis

The present disclosure provides methods and compositions that can be used to treat a subject identified as having myasthenia gravis. In some embodiments, the myasthenia gravis is Class I, Class II (e.g., Class IIa or IIb), Class III (e.g., Class IIIa or IIIb), Class IV (e.g., Class IVa or IVb), or Class V myasthenia gravis. In some embodiments, the myasthenia gravis is Class I myasthenia gravis. In some embodiments, the myasthenia gravis is Class II (e.g., Class IIa or IIb) myasthenia gravis. In some embodiments, the myasthenia gravis is Class III (e.g., Class IIIa or IIIb) myasthenia gravis. In some embodiments, the myasthenia gravis is Class IV (e.g., Class IVa or IVb) myasthenia gravis. In some embodiments, the myasthenia gravis is Class V myasthenia gravis. In some embodiments, the myasthenia gravis is anti-acetylcholine receptor (anti-AchR) positive (e.g., the subject tests positive for anti-AchR antibodies). In some embodiments, the myasthenia gravis is anti-AchR negative (i.e., the subject tests negative for anti-AchR antibodies). In some embodiments, the myasthenia gravis is refractory myasthenia gravis. In some embodiments, the myasthenia gravis is Class IV refractory anti-AchR negative myasthenia gravis. In some embodiments, a subject with myasthenia gravis is determined to have a mean anti-AChR antibody titer of about 50×10⁻⁹ M, 60×10⁻⁹ M, 70×10⁻⁹ M, 80×10⁻⁹ M, 90×10⁻⁹ M, 100×10⁻⁹ M, 200×10⁻⁹ M, or greater. In some embodiments, a subject with myasthenia gravis is positive for one or more non-anti-AChR antibodies that are associated with the disease (e.g., anti-MuSK antibodies, anti-RyR antibodies, anti-striated muscle antibodies, anti-titin antibodies, anti-LRP4 antibodies, etc.). In some embodiments, a subject with myasthenia gravis is positive for one or more antibodies that are associated with Lambert-Eaton syndrome (e.g., anti-VGCC N type antibodies). In some embodiments, a subject may be anti-AchR negative but positive for one or more other autoantibodies (e.g., anti-MuSK antibodies, anti-RyR antibodies, anti-striated muscle antibodies, anti-titin antibodies, anti-LRP4 antibodies, anti-VGCC N type antibodies, etc.). In some embodiments, the autoantibodies are IgG1 antibodies. In some embodiments, a subject with myasthenia gravis is seronegative for antibodies known to be associated with myasthenia gravis.

It is understood that anti-AChR antibodies include three types. Anti-AChR “binding antibodies” bind to the AChR protein on the muscle end plate and activate the complement system; anti-AChR “blocking antibodies” bind to the AChR on the muscle end plate and impairs binding of Ach with the receptor; anti-AChR “modulating antibodies” bind to the AChR on the muscle end plate and cause endocytosis of the receptor. A subject can have one or more types of AChR antibodies. In some embodiments, a subject with myasthenia gravis is positive for anti-AChR binding antibodies. In some embodiments, a subject with myasthenia gravis is negative for anti-AChR binding antibodies. In some embodiments, a subject (e.g., a subject with or suspected to have myasthenia gravis) is determined to be positive for anti-AChR binding antibodies if the antibodies are present at 0.5 nmol/L or greater in serum as measured by quantitative radioimmunoassay, or a suitable alternative assay. In some embodiments, a subject is determined to be negative for anti-AChR binding antibodies if the antibodies are present at 0.0-0.4 nmol/L in serum as measured by quantitative radioimmunoassay, or a suitable alternative assay. In some embodiments, a subject with myasthenia gravis is positive for anti-AChR blocking antibodies. In some embodiments, a subject with myasthenia gravis is negative for anti-AChR blocking antibodies. In some embodiments, a subject (e.g., a subject with or suspected to have myasthenia gravis) is determined to be positive for anti-AChR blocking antibodies if 42% or greater blocking is measured by semi-quantitative flow cytometry, or a suitable alternative assay. In some embodiments, a subject is determined to be negative for anti-AChR blocking antibodies if 0-26% blocking is measured by semi-quantitative flow cytometry, or a suitable alternative assay. In some embodiments, a subject with myasthenia gravis is positive for anti-AChR modulating antibodies. In some embodiments, a subject with myasthenia gravis is negative for anti-AChR modulating antibodies. In some embodiments, a subject (e.g., a subject with or suspected to have myasthenia gravis) is determined to be positive for anti-AChR modulating antibodies if 46% or greater modulating is measured by semi-quantitative flow cytometry, or a suitable alternative assay. In some embodiments, a subject is determined to be negative for anti-AChR modulating antibodies if 0-45% modulating is measured semi-quantitative flow cytometry, or a suitable alternative assay. A plurality of the types of anti-AChR antibodies can be measured, e.g., according to Howard et al., Ann. N.Y. Acad. Sci. (1987) 505:526-38.

The subject suitable for the treatment of the present disclosure can has one or more of the following characteristics: (i) is ≥18 years of age with a diagnosis of generalized MG; (ii) presence of autoantibodies to AChR, MuSK, or LRP4; (iii) Myasthenia Gravis Foundation of America (MGFA) Class IIB-IV; (iv) MG-ADL total score of ≥6 at screening and at predose baseline (i.e., prior to receiving a provided treatment), and a score of ≥2 on one or more individual sub-components for talking, chewing, swallowing, or breathing; (v) failed treatment over 1 year or more with 2 or more immunosuppressive/immunomodulatory therapies or; failed at least 1 immunosuppressive therapy and required chronic plasmapheresis, or IVIG to control symptoms; (vi) on a stable dose of glucocorticoids and/or other immunotherapies for ≥1 month prior to screening for eligibility in receiving a provided treatment (for azathioprine, being on a stable dose for ≥2 months prior); (vii) no change in dose of acetylcholinesterase inhibitors for ≥2 weeks prior to screening for eligibility in receiving a provided treatment; (viii) no use of IVIG or PLEX within 4 weeks of pre-dose baseline (i.e., prior to receiving a provided treatment); or (ix) Eastern Cooperative Oncology Group (ECOG) performance status of 0 or 1. In certain embodiments, the subject has all the characteristics above in this paragraph.

The present disclosure provides, among other things, methods and compositions for reducing the number of B cells in a tissue in a subject having myasthenia gravis. The present disclosure also provides engineered T cells (e.g., T cells engineered to express any CAR described herein) and method of making engineered T cells for use in treating myasthenia gravis.

The present disclosure appreciates that B cells express a wide array of cell surface molecules during their differentiation and proliferation, e.g., CD19. CD19 is widely expressed on B cells during all phases of B cell development from pro-B cells to plasmablasts. The present disclosure further appreciates, that because of the ubiquity of CD19 on B cells, CD19 can function as a therapeutic target for certain provided methods and compositions (e.g., methods and compositions for treating myasthenia gravis). Accordingly, in some embodiments, provided herein are methods and compositions for reducing the number of B cells in a subject (e.g., in a tissue of a subject) via targeting of CD19. In some embodiments, the present disclosure provides for methods and compositions for treating a subject having myasthenia gravis via targeting of CD19. In some embodiments, the present disclosure provides for engineered T cells that target CD19. In some embodiments, the present disclosure provides for engineered nucleic acids that express one or more polypeptides that target CD19. In many embodiments of the present disclosure a CAR that binds to CD19 is used to target cells that express CD19 (e.g., B cells).

### Chimeric Antigen Receptors

In some embodiments, a chimeric antigen receptor (CAR) of the present disclosure comprises an extracellular domain, a transmembrane domain, and an intracellular domain. In some embodiments, an extracellular domain is or comprises an antigen-binding domain (e.g., a CD19 binding domain, such as an anti-CD19 scFv). In some embodiments, the extracellular domain is or comprises a means for binding CD19. In some embodiments, a transmembrane domain is or comprises a transmembrane domain or functional fragment thereof derived from any suitable cell membrane-associated polypeptide, e.g., obtained from a membrane-binding polypeptide or transmembrane polypeptide. In some embodiments, a transmembrane domain is or comprises a transmembrane domain or functional fragment thereof derived from a T cell receptor alpha chain, a T cell receptor beta chain, a CD3 zeta chain, a CD28 polypeptide, or a CD8 polypeptide (e.g., a CD8α polypeptide). In some embodiments, an intracellular domain is or comprises an intracellular signaling domain (e.g., any of intracellular signaling domains described herein, e.g., derived from a CD28 or CD3 polypeptide). In some embodiments, an intracellular signaling domain comprises one or more signaling sequences or motifs. In some embodiments, one or more signaling sequences, or signaling motifs, are essential for the functional signaling capacity of a polypeptide (e.g., an intracellular signaling domain). In some embodiments, a signaling sequence is a sequence derived from a CD3 polypeptide (e.g., a CD3 zeta polypeptide). In some embodiments, a signaling sequence is derived from a CD28 polypeptide. In some embodiments, a signaling sequence is or comprises a co-stimulatory domain (e.g., any co-stimulatory domain described herein, e.g., derived from a CD28 polypeptide). In some embodiments, a CAR of the present disclosure is a human CAR.

### Extracellular Domain

In some embodiments, an extracellular domain used in accordance with the present disclosure comprises an antigen-binding domain (e.g., any antigen-binding domain described herein). The extracellular domain can be or include a means for binding CD19 (e.g., human CD19). In some embodiments, an antigen-binding domain is or comprises an antibody sequence (e.g., an immunoglobulin) or antigen-binding fragment thereof (e.g., any antibody or antigen-binding fragment thereof described herein). Anticalins or other alternative scaffolds are also contemplated.

In some embodiments, the antigen-binding domain comprises one or more Fab, Fab′, F(ab′)₂, Fv, domain antibody (dAb), single-chain antibody (scFv), chimeric antibody, diabody, triabody, tetrabody, scAb, or single domain antibody (e.g., VHH or VNAR) polypeptide sequences. In some embodiments, the antigen-binding domain comprises at least a portion of an immunoglobulin that is sufficient to confer specific antigen-binding to a polypeptide (e.g., an antibody fragment comprising an antigen-binding portion). In some embodiments, the antigen-binding domain comprises an scFv. In some embodiments, the scFv comprises a VH and VL domain of an antibody. In some embodiments, the scFv comprises a spacer sequence between the VH and the VL. In some embodiments, the scFv comprises a spacer sequence as set forth in SEQ ID NO: 9 between the VH and the VL. In some embodiments, the antigen-binding domain is humanized, or fully human (e.g., derived from a suitable human polypeptide). Exemplary methods of generating fully human antibodies are described in Lu et al., (2020) J. Biomed. Sci. (2020) 27(1):1.

In some embodiments, the antigen-binding domain binds to a target antigen (e.g., a polypeptide). In some embodiments, the antigen-binding domain binds specifically to a target antigen (e.g., a polypeptide). In some embodiments, the antigen-binding domain binds to a CD19 polypeptide (e.g., a CD19 polypeptide present at the surface of a cell, e.g., a B cell). In some embodiments, the antigen-binding domain binds specifically to a CD19 polypeptide. In some embodiments, the antigen-binding domain comprises an antibody, or antigen-binding fragment thereof, that binds to a CD19 polypeptide. In some embodiments, the antigen-binding domain comprises a scFv sequence that binds to a CD19 polypeptide (e.g., an anti-CD19 scFv).

It is generally understood that CD19 expression is largely restricted to B lymphocytes. CD19 has two N-terminal extracellular Ig-like domains separated by a non-Ig-like domain, a hydrophobic transmembrane domain, and a large C-terminal cytoplasmic domain. The CD19 protein forms a complex with several membrane proteins including complement receptor type 2 (CD21) and tetraspanin (CD81) and this complex reduces the threshold for antigen-initiated B cell activation. Activation of this B-cell antigen receptor complex activates the phosphatidylinositol 3-kinase signaling pathway and the subsequent release of intracellular stores of calcium ions. An example of a human CD19 polypeptide sequence includes, without limitation, NCBI reference sequence: NP_001171569.1, and fragments and derivatives thereof.

In some embodiments, an antigen-binding domain comprises a variable region of an anti-CD19 antibody. In some embodiments, an antigen-binding domain comprises a variable region of an anti-CD19 monoclonal antibody. In some embodiments, an antigen-binding domain comprises a variable region of a mouse or human anti-CD19 monoclonal antibody. An anti-CD19 monoclonal antibody can be obtained or derived from a subject (e.g., a mouse, a rat, a rabbit, a human, etc.) using any suitable method. In some embodiments, an antigen-binding domain comprises a light chain variable region and a heavy chain variable region of a mouse, human, or humanized anti-CD19 monoclonal antibody. In some embodiments, an antigen-binding domain comprises a light chain variable region of a mouse, human, or humanized anti-CD19 monoclonal antibody. In some embodiments, an antigen-binding domain comprises a heavy chain variable region of a mouse, human, or humanized anti-CD19 monoclonal antibody. The 47G4 antibody (described in U.S. Patent Application Publication No. 2010/0104509, which is incorporated herein by reference in its entirety) is one example of a human anti-CD19 monoclonal antibody that can be used in accordance with the present disclosure.

In some embodiments, the antigen-binding domain that binds CD19 comprises a heavy chain variable domain and a light chain variable domain, the heavy chain variable domain comprising CDR1, CDR2, and CDR3 amino acid sequences having at least 80%, at least 85%, at least 90%, at least 95%, at least 96%, at least 97%, at least 98%, at least 99%, or greater sequence identity to SEQ ID NO: 1, SEQ ID NO: 2, and SEQ ID NO: 3, respectively, and the light chain variable domain comprising CDR1, CDR2, and CDR3 amino acid sequences having at least 80%, at least 85%, at least 90%, at least 95%, at least 96%, at least 97%, at least 98%, at least 99%, or greater sequence identity to SEQ ID NO: 4, SEQ ID NO: 5, and SEQ ID NO: 6, respectively. In some embodiments, the antigen-binding domain that binds CD19 comprises a heavy chain variable domain and a light chain variable domain, the heavy chain variable domain comprising CDR1, CDR2, and CDR3 amino acid sequences as set forth in SEQ ID NO: 1, SEQ ID NO: 2, and SEQ ID NO: 3, respectively, and the light chain variable domain comprising CDR1, CDR2, and CDR3 amino acid sequences as set forth in SEQ ID NO: 4, SEQ ID NO: 5, and SEQ ID NO: 6, respectively.

In some embodiments, the antigen-binding domain that binds CD19 comprises a heavy chain variable domain and a light chain variable domain, the heavy chain variable domain comprising CDR1, CDR2, and CDR3 amino acid sequences having at least 80%, at least 85%, at least 90%, at least 95%, at least 96%, at least 97%, at least 98%, at least 99%, or greater sequence identity to SEQ ID NO: 25, SEQ ID NO: 26, and SEQ ID NO: 3, respectively, and the light chain variable domain comprising CDR1, CDR2, and CDR3 amino acid sequences having at least 80%, at least 85%, at least 90%, at least 95%, at least 96%, at least 97%, at least 98%, at least 99%, or greater sequence identity to SEQ ID NO: 4, SEQ ID NO: 5, and SEQ ID NO: 6, respectively. In some embodiments, the antigen-binding domain that binds CD19 comprises a heavy chain variable domain and a light chain variable domain, the heavy chain variable domain comprising CDR1, CDR2, and CDR3 amino acid sequences as set forth in SEQ ID NO: 25, SEQ ID NO: 26, and SEQ ID NO: 3, respectively, and the light chain variable domain comprising CDR1, CDR2, and CDR3 amino acid sequences as set forth in SEQ ID NO: 4, SEQ ID NO: 5, and SEQ ID NO: 6, respectively.

In some embodiments, the antigen-binding domain that binds CD19 comprises a heavy chain variable domain, the heavy chain variable domain comprising CDR1, CDR2, and CDR3 amino acid sequences having at least 80%, at least 85%, at least 90%, at least 95%, at least 96%, at least 97%, at least 98%, at least 99%, or greater sequence identity to SEQ ID NO: 1, SEQ ID NO: 2, and SEQ ID NO: 3, respectively. In some embodiments, the antigen-binding domain that binds CD19 comprises a heavy chain variable domain, the heavy chain variable domain comprising CDR1, CDR2, and CDR3 amino acid sequences as set forth in SEQ ID NO: 1, SEQ ID NO: 2, and SEQ ID NO: 3, respectively.

In some embodiments, the antigen-binding domain that binds CD19 comprises a heavy chain variable domain, the heavy chain variable domain comprising CDR1, CDR2, and CDR3 amino acid sequences having at least 80%, at least 85%, at least 90%, at least 95%, at least 96%, at least 97%, at least 98%, at least 99%, or greater sequence identity to SEQ ID NO: 25, SEQ ID NO: 26, and SEQ ID NO: 3, respectively. In some embodiments, the antigen-binding domain that binds CD19 comprises a heavy chain variable domain, the heavy chain variable domain comprising CDR1, CDR2, and CDR3 amino acid sequences as set forth in SEQ ID NO: 25, SEQ ID NO: 26, and SEQ ID NO: 3, respectively.

In some embodiments, the antigen-binding domain that binds CD19 comprises a light chain variable domain, the light chain variable domain comprising CDR1, CDR2, and CDR3 amino acid sequences having at least 80%, at least 85%, at least 90%, at least 95%, at least 96%, at least 97%, at least 98%, at least 99%, or greater sequence identity to SEQ ID NO: 4, SEQ ID NO: 5, and SEQ ID NO: 6, respectively. In some embodiments, the antigen-binding domain that binds CD19 comprises a light chain variable domain, the light chain variable domain comprising CDR1, CDR2, and CDR3 amino acid sequences as set forth in SEQ ID NO: 4, SEQ ID NO: 5, and SEQ ID NO: 6, respectively.

In some embodiments, the antigen-binding domain that binds CD19 comprises a heavy chain variable domain and a light chain variable domain, the heavy chain variable domain comprising an amino acid sequence with at least 70%, at least 75%, at least 80%, at least 85%, at least 90%, at least 95%, at least 96%, at least 97%, at least 98%, at least 99%, or greater sequence identity to SEQ ID NO: 7, and the light chain variable domain comprising an amino acid sequence with at least 70%, at least 75%, at least 80%, at least 85%, at least 90%, at least 95%, at least 96%, at least 97%, at least 98%, at least 99%, or greater sequence identity to SEQ ID NO: 8. In some embodiments, the antigen-binding domain that binds CD19 comprises a heavy chain variable domain and a light chain variable domain, the heavy chain variable domain comprising an amino acid sequence with at least 95% sequence identity to SEQ ID NO: 7, and the light chain variable domain comprising an amino acid sequence with at least 95% sequence identity to SEQ ID NO: 8. In some embodiments, the antigen-binding domain that binds CD19 comprises a heavy chain variable domain and a light chain variable domain, the heavy chain variable domain comprising an amino acid sequence with at least 90% sequence identity to SEQ ID NO: 7, and the light chain variable domain comprising an amino acid sequence with at least 90% sequence identity to SEQ ID NO: 8. In some embodiments, the antigen-binding domain that binds CD19 comprises a heavy chain variable domain and a light chain variable domain, the heavy chain variable domain comprising an amino acid sequence as set forth in SEQ ID NO: 7, and the light chain variable domain comprising an amino acid sequence as set forth in SEQ ID NO: 8.

In some embodiments, the antigen-binding domain that binds CD19 comprises a heavy chain variable domain, the heavy chain variable domain comprising an amino acid sequence with at least 70%, at least 75%, at least 80%, at least 85%, at least 90%, at least 95%, at least 96%, at least 97%, at least 98%, at least 99%, or greater sequence identity to SEQ ID NO: 7. In some embodiments, the antigen-binding domain that binds CD19 comprises a heavy chain variable domain, the heavy chain variable domain comprising an amino acid sequence with at least 90% sequence identity to SEQ ID NO: 7. In some embodiments, the antigen-binding domain that binds CD19 comprises a heavy chain variable domain, the heavy chain variable domain comprising an amino acid sequence with at least 95% sequence identity to SEQ ID NO: 7. In some embodiments, the antigen-binding domain that binds CD19 comprises a heavy chain variable domain, the heavy chain variable domain comprising an amino acid sequence as set forth in SEQ ID NO: 7.

In some embodiments, the antigen-binding domain that binds CD19 comprises a light chain variable domain, the light chain variable domain comprising an amino acid sequence with at least 70%, at least 75%, at least 80%, at least 85%, at least 90%, at least 95%, at least 96%, at least 97%, at least 98%, at least 99%, or greater sequence identity to SEQ ID NO: 8. In some embodiments, the antigen-binding domain that binds CD19 comprises a light chain variable domain, the light chain variable domain comprising an amino acid sequence with at least 90% sequence identity to SEQ ID NO: 8. In some embodiments, the antigen-binding domain that binds CD19 comprises a light chain variable domain, the light chain variable domain comprising an amino acid sequence with at least 95% sequence identity to SEQ ID NO: 8. In some embodiments, the antigen-binding domain that binds CD19 comprises a light chain variable domain, the light chain variable domain comprising an amino acid sequence as set forth in SEQ ID NO: 8.

In some embodiments, the antigen-binding domain that binds CD19 comprises a spacer sequence between two domains or components. In some embodiments, an antigen-biding domain comprises a spacer sequence between a heavy chain variable domain and a light chain variable domain. In some embodiments, a spacer comprises a sequence as set forth in SEQ ID NO: 9.

In some embodiments, the antigen-binding domain that binds CD19 comprises an amino acid sequence having at least 70%, at least 75%, at least 80%, at least 85%, at least 90%, at least 95%, at least 96%, at least 97%, at least 98%, at least 99%, or greater sequence identity to SEQ ID NO: 17. In some embodiments, the antigen-binding domain that binds CD19 comprises an amino acid sequence having at least 90% sequence identity to SEQ ID NO: 17. In some embodiments, the antigen-binding domain that binds CD19 comprises an amino acid sequence as set forth in SEQ ID NO: 17.

In some embodiments, the antigen-binding domain that binds CD19 is encoded by a nucleic acid sequence having at least 70%, at least 75%, at least 80%, at least 85%, at least 90%, at least 95%, at least 96%, at least 97%, at least 98%, at least 99%, or greater sequence identity to SEQ ID NO: 18. In some embodiments, the antigen-binding domain that binds CD19 is encoded by a nucleic acid sequence having at least 90% sequence identity to SEQ ID NO: 18. In some embodiments, an antigen-binding domain is encoded a nucleic acid sequence as set forth in SEQ ID NO: 18.

Other antigen-binding domains that bind CD19 can also be included in the CAR disclosed herein. Exemplary antigen-binding domains are described in International Application Publication No. WO2017062952 and U.S. Application Publication No. US20220220200.

In some embodiments, the antigen-binding domain that binds CD19 comprises a heavy chain variable domain and a light chain variable domain, the heavy chain variable domain comprising CDR1, CDR2, and CDR3 amino acid sequences as set forth in SEQ ID NOs: 35, 36, and 37, respectively, and the light chain variable domain comprising CDR1, CDR2, and CDR3 amino acid sequences as set forth in SEQ ID NOs: 38, 39, and 40, respectively. In some embodiments, the heavy chain variable domain comprises an amino acid sequence with at least 70%, at least 75%, at least 80%, at least 85%, at least 90%, at least 95%, at least 96%, at least 97%, at least 98%, at least 99%, or greater sequence identity to SEQ ID NO: 29, and the light chain variable domain comprises an amino acid sequence with at least 70%, at least 75%, at least 80%, at least 85%, at least 90%, at least 95%, at least 96%, at least 97%, at least 98%, at least 99%, or greater sequence identity to SEQ ID NO: 30.

In some embodiments, the antigen-binding domain that binds CD19 comprises a heavy chain variable domain and a light chain variable domain, the heavy chain variable domain comprising CDR1, CDR2, and CDR3 amino acid sequences as set forth in SEQ ID NOs: 41, 42, and 43, respectively, and the light chain variable domain comprising CDR1, CDR2, and CDR3 amino acid sequences as set forth in SEQ ID NOs: 44, 45, and 46, respectively. In some embodiments, the heavy chain variable domain comprises an amino acid sequence with at least 70%, at least 75%, at least 80%, at least 85%, at least 90%, at least 95%, at least 96%, at least 97%, at least 98%, at least 99%, or greater sequence identity to SEQ ID NO: 31, and the light chain variable domain comprises an amino acid sequence with at least 70%, at least 75%, at least 80%, at least 85%, at least 90%, at least 95%, at least 96%, at least 97%, at least 98%, at least 99%, or greater sequence identity to SEQ ID NO: 32.

In some embodiments, the antigen-binding domain that binds CD19 comprises a heavy chain variable domain and a light chain variable domain, the heavy chain variable domain comprising CDR1, CDR2, and CDR3 amino acid sequences as set forth in SEQ ID NOs: 47, 48, and 49, respectively, and the light chain variable domain comprising CDR1, CDR2, and CDR3 amino acid sequences as set forth in SEQ ID NOs: 50, 51, and 52, respectively. In some embodiments, the heavy chain variable domain comprises an amino acid sequence with at least 70%, at least 75%, at least 80%, at least 85%, at least 90%, at least 95%, at least 96%, at least 97%, at least 98%, at least 99%, or greater sequence identity to SEQ ID NO: 33, and the light chain variable domain comprises an amino acid sequence with at least 70%, at least 75%, at least 80%, at least 85%, at least 90%, at least 95%, at least 96%, at least 97%, at least 98%, at least 99%, or greater sequence identity to SEQ ID NO: 34.

In some embodiments, the extracellular domain of the CAR further comprises a hinge region. In some embodiments, a hinge region is positioned between (e.g., links together), an extracellular domain and a transmembrane domain. In some embodiments, the hinge region is a short sequence of amino acids that can facilitate structural flexibility between polypeptide domains, e.g., between an extracellular domain and a transmembrane domain (see, e.g. Woof et al., Nat. Rev. Immunol. 4(2):89-99 (2004)). In some embodiments, a hinge region may include all, or a portion of, an extracellular region of any suitable transmembrane protein (e.g., CD8α).

In some embodiments, the hinge region is derived from a CD8α protein or a CD28 protein. In some embodiments, a hinge region is derived from a CD8α protein. In some embodiments, the hinge region is derived from a CD28 protein. In some embodiments, a hinge region is or comprises a hinge region or functional fragment thereof from a CD28 protein. In some embodiments, the hinge region is or comprises a hinge region or functional fragment thereof from a CD8α protein. In some embodiments, the hinge region is derived from a human CD8α protein or a human CD28 protein. In some embodiments, the hinge region is derived from a human CD8α protein. In some embodiments, the hinge region is derived from a human CD28 protein. In some embodiments, the hinge region is or comprises a hinge region or functional fragment thereof from a human CD28 protein. In some embodiments, the hinge region is or comprises a hinge region or functional fragment thereof from a human CD8α protein.

In some embodiments, a hinge region comprises an amino acid sequence having at least 70%, at least 75%, at least 80%, at least 85%, at least 90%, at least 95%, at least 96%, at least 97%, at least 98%, at least 99%, or greater sequence identity to SEQ ID NO: 28. In some embodiments, a hinge region comprises an amino acid sequence as set forth in SEQ ID NO: 28.

In some embodiments, a hinge region is derived from the same polypeptide as a transmembrane domain. In some embodiments, a hinge region and a transmembrane domain are derived from a CD8 polypeptide. In some embodiments, a hinge region and a transmembrane domain are derived from a CD8α polypeptide. In some embodiments, a hinge region and transmembrane domain comprise an amino acid sequence having at least 70%, at least 75%, at least 80%, at least 85%, at least 90%, at least 95%, at least 96%, at least 97%, at least 98%, at least 99%, or greater sequence identity to SEQ ID NO: 19. In some embodiments, a hinge region and transmembrane domain comprise an amino acid sequence having at least 90% sequence identity to SEQ ID NO: 19. In some embodiments, a hinge region and transmembrane domain comprise an amino acid sequence as set forth in SEQ ID NO: 19.

In some embodiments, a hinge region and transmembrane domain are encoded by nucleic acid sequence having at least 70%, at least 75%, at least 80%, at least 85%, at least 90%, at least 95%, at least 96%, at least 97%, at least 98%, at least 99%, or greater sequence identity to SEQ ID NO: 20. In some embodiments, a hinge region and transmembrane domain are encoded by a nucleic acid sequence having at least 90% sequence identity to SEQ ID NO: 20. In some embodiments, a hinge region and transmembrane domain are encoded by a nucleic acid sequence as set forth in SEQ ID NO: 20.

### Transmembrane Domain

In some embodiments, the transmembrane domain of the CAR is derived from a natural source (e.g., a natural or wild-type polypeptide). In some embodiments, the transmembrane domain, as used in accordance with the present disclosure, is derived from any suitable transmembrane protein or polypeptide known in the art. In some embodiments, a transmembrane domain is derived from a CD3 epsilon polypeptide, a CD4 polypeptide, a CD5 polypeptide, a CD8 polypeptide, a CD9 polypeptide, a CD16 polypeptide, a CD22 polypeptide, a CD28 polypeptide, a CD33 polypeptide, a CD37 polypeptide, a CD45 polypeptide, a CD64 polypeptide, a CD80 polypeptide, a CD86 polypeptide, a CD134 polypeptide, a CD137 polypeptide, a CD154 polypeptide, a T cell receptor alpha chain polypeptide, a T cell receptor beta chain polypeptide, a T cell receptor zeta chain polypeptide, or any combination thereof. In some embodiments, a transmembrane is or comprises a transmembrane domain or functional fragment thereof from a CD3 epsilon polypeptide, a CD4 polypeptide, a CD5 polypeptide, a CD8 polypeptide, a CD9 polypeptide, a CD16 polypeptide, a CD22 polypeptide, a CD28 polypeptide, a CD33 polypeptide, a CD37 polypeptide, a CD45 polypeptide, a CD64 polypeptide, a CD80 polypeptide, a CD86 polypeptide, a CD134 polypeptide, a CD137 polypeptide, a CD154 polypeptide, a T cell receptor alpha chain polypeptide, a T cell receptor beta chain polypeptide, a T cell receptor zeta chain polypeptide, or any derivatives thereof and/or any combination thereof. In some embodiments, a transmembrane is synthetically derived, or engineered. In some embodiments, a synthetically derived or engineered transmembrane domain comprises predominantly hydrophobic residues (e.g., leucine, valine, etc.). In some embodiments, an engineered transmembrane domain is or comprises any engineered transmembrane domain known in the field.

The present disclosure appreciates that CD8 is a transmembrane glycoprotein that functions as a co-receptor for the T-cell receptor (TCR), and is expressed primarily on the surface of T-cells (e.g., cytotoxic T-cells). The most common form of CD8 exists as a dimer composed of a CD8α and CD8β chain. In some embodiments, a transmembrane domain is derived from a CD8α protein. In some embodiments, a transmembrane protein comprises an amino acid sequence having at least 70%, at least 75%, at least 80%, at least 85%, at least 90%, at least 95%, at least 96%, at least 97%, at least 98%, at least 99%, or greater sequence identity to SEQ ID NO: 11. In some embodiments, a transmembrane protein comprises an amino acid sequence as set forth in SEQ ID NO: 11.

The present disclosure further appreciates that CD28 is expressed on T-cells and provides co-stimulatory signals required for T-cell activation. CD28 is the receptor for CD80 (B7.1) and CD86 (B7.2). In some embodiments, a CAR of the present disclosure comprises a CD28 transmembrane domain. In some embodiments, the transmembrane protein comprises an amino acid sequence having at least 70%, at least 75%, at least 80%, at least 85%, at least 90%, at least 95%, at least 96%, at least 97%, at least 98%, at least 99%, or greater sequence identity to SEQ ID NO: 12. In some embodiments, a transmembrane protein comprises an amino acid sequence as set forth in SEQ ID NO: 12.

### Intracellular Signaling Domain

In some embodiments, an intracellular signaling domain of the CAR disclosed herein is derived from a polypeptide found in humans (e.g., an intracellular signaling domain or fragment thereof found in any suitable human polypeptide). In some embodiments, the intracellular signaling domain provided herein is derived from a 4-1BB polypeptide, a B7-H3 polypeptide, a CD2 polypeptide, a CD3 gamma polypeptide, a CD3 delta polypeptide, a CD3 zeta polypeptide, a CD7 polypeptide, a CD27 polypeptide, a CD28 polypeptide, a CD30 polypeptide, a CD40 polypeptide, an FcεRI polypeptide (e.g., an FcεRI gamma chain polypeptide), an FcγRI polypeptide, LIGHT polypeptide, NKG2C polypeptide, OX40 polypeptide, PD-1 polypeptide, or any derivatives thereof or any combination thereof. In some embodiments, the intracellular signaling domain is derived from a CD3 zeta polypeptide. In some embodiments, the intracellular signaling domain is derived from a CD28 polypeptide. In some embodiments, the intracellular signaling domain is derived from a CD28 polypeptide and a CD3 zeta polypeptide.

In some embodiments, the intracellular signaling domain comprises at least one intracellular signaling domain or functional fragment thereof from a 4-1BB polypeptide, a B7-H3 polypeptide, a CD2 polypeptide, a CD3 gamma polypeptide, a CD3 delta polypeptide, a CD3 zeta polypeptide, a CD7 polypeptide, a CD27 polypeptide, a CD28 polypeptide, a CD30 polypeptide, a CD40 polypeptide, an FcεRI polypeptide (e.g., an FcεRI gamma chain polypeptide), an FcγRI polypeptide, LIGHT polypeptide, NKG2C polypeptide, OX40 polypeptide, PD-1 polypeptide, or any derivatives thereof or any combination thereof. In some embodiments, the intracellular signaling domain comprises an intracellular signaling domain or functional fragment thereof from a CD3 zeta polypeptide. In some embodiments, the intracellular signaling domain comprises an intracellular signaling domain or functional fragment thereof from a CD28 polypeptide. In some embodiments, the intracellular signaling domain comprises an intracellular signaling domain or functional fragment thereof from a CD28 polypeptide and an intracellular signaling domain or functional fragment thereof from a CD3 zeta polypeptide. In some embodiments, the intracellular signaling domain comprises, from N-terminus to C-terminus, an intracellular signaling domain or functional fragment thereof from a CD28 polypeptide and an intracellular signaling domain or functional fragment thereof from a CD3 zeta polypeptide.

In some embodiments, the intracellular signaling domain of the present disclosure comprises at least one signaling sequence from a 4-1BB polypeptide, a B7-H3 polypeptide, a CD2 polypeptide, a CD3 gamma polypeptide, a CD3 delta polypeptide, a CD3 zeta polypeptide, a CD7 polypeptide, a CD27 polypeptide, a CD28 polypeptide, a CD30 polypeptide, a CD40 polypeptide, an FCεRI polypeptide (e.g., an FCεRI gamma chain polypeptide), an FcγRI polypeptide, LIGHT polypeptide, NKG2C polypeptide, OX40 polypeptide, PD-1 polypeptide, or any combination thereof. In some embodiments, the intracellular signaling domain comprises at least one signaling sequence from a CD3 zeta polypeptide. In some embodiments, the intracellular signaling domain comprises at least one signaling sequence from a CD28 polypeptide. In some embodiments, the intracellular signaling domain comprises at least one signaling sequence from a CD28 polypeptide and at least one signaling sequence from a CD3 zeta polypeptide. In some embodiments, the intracellular signaling domain comprises, from N-terminus to C-terminus, at least one signaling sequence from a CD28 polypeptide and at least one signaling sequence from a CD3 zeta polypeptide.

In some embodiments, an intracellular signaling domain is or comprises at least one signaling sequence or signaling motif. In some embodiments, a signaling sequence (or signaling motif) comprises one or more (e.g., two, three, four, five, or more) co-stimulatory domains (e.g., any co-stimulatory domain described herein). In some embodiments, a signaling sequence comprises one co-stimulatory domain. In some embodiments, a signaling sequence comprises two co-stimulatory domains. In some embodiments, a signaling sequence comprises three co-stimulatory domains. In some embodiments, a signaling sequence comprises two or more of the same co-stimulatory domains. In some embodiments, a signaling sequence comprises two or more different co-stimulatory domains.

In some embodiments, a signaling sequence as used in accordance with the present disclosure is or comprises one or more immunoreceptor tyrosine-based activation motifs (ITAMs). In some embodiments, a signal sequence is or comprises a consensus sequence of YXXL/I, where Y is a tyrosine residue, L/I is a leucine or isoleucine residue, and X is any amino acid residue. In some embodiments, a signal sequence is or comprises a consensus sequences of YXXL/IX₍₆₋₈₎YXXL (SEQ ID NO: 54), where Y is a tyrosine residue, L/I is a leucine or isoleucine residue, and X is any amino acid residue. In some embodiments, a signaling sequence comprises a YNMN (SEQ ID NO: 53) motif. In some embodiments, a signaling sequence comprises at least one ITAM sequence from a CD3 polypeptide (e.g., a CD3 zeta polypeptide). In some embodiments, a signaling sequence comprises at least one ITAM sequence from a CD28 polypeptide.

It is understood that the most common intracellular signaling domain used in CAR therapies is an intracellular signaling domain of CD3 zeta (CD3ζ). CD3 zeta associates with T cell receptors to produce a signal and contains ITAMs. In some embodiments, an intracellular signaling domain is or comprises a CD3 zeta intracellular signaling domain. In some embodiments, an intracellular signaling domain comprises an intracellular signaling domain or a functional fragment thereof from a CD3 zeta polypeptide.

In some embodiments, an intracellular signaling domain comprises an amino acid sequence having at least 70%, at least 75%, at least 80%, at least 85%, at least 90%, at least 95%, at least 96%, at least 97%, at least 98%, at least 99%, or greater sequence identity to SEQ ID NO: 23. In some embodiments, an intracellular signaling domain comprises an amino acid sequence as set forth in SEQ ID NO: 23.

In some embodiments, an intracellular signaling domain is encoded by nucleic acid sequence having at least 70%, at least 75%, at least 80%, at least 85%, at least 90%, at least 95%, at least 96%, at least 97%, at least 98%, at least 99%, or greater sequence identity to SEQ ID NO: 24. In some embodiments, an intracellular signaling domain is encoded by a nucleic acid sequence as set forth in SEQ ID NO: 24.

In some embodiments, an intracellular signaling domain comprises a CD28 intracellular signaling domain. In some embodiments, an intracellular signaling domain comprises an intracellular signaling domain or a functional fragment thereof from a CD28 polypeptide. In some embodiments, a CD28 polypeptide intracellular signaling domain or functional fragment thereof comprises a co-stimulatory domain.

In some embodiments, an intracellular signaling domain disclosed herein comprises an amino acid sequence having at least 70%, at least 75%, at least 80%, at least 85%, at least 90%, at least 95%, at least 96%, at least 97%, at least 98%, at least 99%, or greater sequence identity to SEQ ID NO: 21. In some embodiments, the intracellular signaling domain comprises an amino acid sequence as set forth in SEQ ID NO: 21.

In some embodiments, an intracellular signaling domain is encoded by nucleic acid sequence having at least 70%, at least 75%, at least 80%, at least 85%, at least 90%, at least 95%, at least 96%, at least 97%, at least 98%, at least 99%, or greater sequence identity to SEQ ID NO: 22. In some embodiments, an intracellular signaling domain is encoded by a nucleic acid sequence as set forth in SEQ ID NO: 22.

### Chimeric Antigen Receptors (CARs)

In some embodiments, a CAR of the present disclosure comprises an extracellular domain, a hinge domain, a transmembrane domain, and an intracellular signaling domain. In some embodiments, a CAR of the present disclosure comprises a signal peptide sequence (also referred to as a targeting signal, localization signal, localization sequence, leader sequence, or leader peptide), an extracellular domain, a hinge domain, a transmembrane domain, and an intracellular signaling domain. In some embodiments, a CAR of the present disclosure comprises, from N-terminus to C-terminus, an extracellular domain, a hinge domain, a transmembrane domain, and an intracellular signaling domain. In some embodiments, a CAR of the present disclosure comprises, from N-terminus to C-terminus, a signal peptide sequence, an extracellular domain, a hinge domain, a transmembrane domain, and an intracellular signaling domain. In some embodiments, the signal peptide sequence is cleaved from the CAR during or after its insertion into a membrane (e.g., ER membrane) during synthesis of the CAR protein. In some embodiments, domains or components (e.g., extracellular domains, hinge regions, transmembrane domains, intracellular signaling domains, etc.) of a CAR are directly linked, or are contiguous. In some embodiments, domains or components of a CAR are not-directly linked, or are non-contiguous.

In some embodiments, a CAR as described herein comprises an intracellular signaling domain, wherein the intracellular signaling domain comprises: (a) a CD3 zeta intracellular signaling domain or functional fragment thereof; and (b) at least one of a 4-1BB, an OX40, or a CD28 intracellular signaling domain or functional fragment thereof. In some embodiments, a 4-1BB intracellular signaling domain or functional fragment thereof, an OX40 intracellular signaling domain, and/or a CD28 intracellular signaling domain or functional fragment thereof is or comprises a co-stimulatory domain.

In some embodiments, a CAR of the present disclosure comprises: (a) a CD28 transmembrane domain; and (b) an intracellular signaling domain comprising: (i) a CD3ζ intracellular signaling domain or functional fragment thereof; and (ii) a CD28 intracellular signaling domain or functional fragment thereof. In some embodiments, a CD28 intracellular signaling domain or functional fragment thereof is or comprises a CD28 co-stimulatory domain.

In some embodiments, a CAR of the present disclosure comprises: (a) a CD8α transmembrane domain; (b) an intracellular signaling domain comprising: (i) a CD3ζ intracellular signaling domain or functional fragment thereof; and (ii) a CD28, an FcεRI gamma chain, and/or a 4-1BB intracellular signaling domain or functional fragment thereof. In some embodiments, a CAR of the present disclosure comprises: (a) a CD8α transmembrane domain; (b) an intracellular signaling domain comprising: (i) a CD3ζ intracellular signaling domain or functional fragment thereof; and (ii) a CD28, an FcεCRI gamma chain, and a 4-1BB intracellular signaling domain or functional fragment thereof. In some embodiments, a CAR of the present disclosure comprises: (a) a CD8α transmembrane domain; (b) an intracellular signaling domain comprising: (i) a CD3ζ intracellular signaling domain or functional fragment thereof; and (ii) an FcεRI gamma chain intracellular signaling domain or functional fragment thereof. In some embodiments, a CAR of the present disclosure comprises: (a) a CD8α transmembrane domain; (b) an intracellular signaling domain comprising: (i) a CD3ζ intracellular signaling domain or functional fragment thereof; and (ii) a 4-1BB intracellular signaling domain or functional fragment thereof. In some embodiments, a CD28 intracellular signaling domain or functional fragment thereof is or comprises a CD28 co-stimulatory domain. In some embodiments, a FcεCRI intracellular signaling domain or functional fragment thereof is or comprises a FcεRI co-stimulatory domain. In some embodiments, a 4-1BB intracellular signaling domain or functional fragment thereof is or comprises a 4-1BB co-stimulatory domain.

In some embodiments, a CAR of the present disclosure comprises (a) a CD8α transmembrane domain, and (b) an intracellular signaling domain comprising: (i) a CD3ζ intracellular signaling domain or functional fragment thereof, and (ii) a CD27 and/or a CD28 intracellular signaling domain or functional fragment thereof. In some embodiments, a CD27 intracellular signaling domain or functional fragment thereof is or comprises a CD27 co-stimulatory domain. In some embodiments, a CD28 intracellular signaling domain or functional fragment thereof is or comprises a CD28 co-stimulatory domain.

In some embodiments, a CAR of the present disclosure comprises (a) a CD28 transmembrane domain, and (b) an intracellular signaling domain comprising: (i) a CD3ζ intracellular signaling domain or functional fragment thereof; and (ii) a CD27, a 4-1BB, and/or an FcεRI gamma chain intracellular signaling domain or functional fragment thereof. In some embodiments, a CAR of the present disclosure comprises (a) a CD28 transmembrane domain, and (b) an intracellular signaling domain comprising: (i) a CD3ζ intracellular signaling domain or functional fragment thereof; and (ii) a CD27, a 4-1BB, and an FcεCRI gamma chain intracellular signaling domain or functional fragment thereof. In some embodiments, a CAR of the present disclosure comprises (a) a CD28 transmembrane domain, and (b) an intracellular signaling domain comprising: (i) a CD3ζ intracellular signaling domain or functional fragment thereof; and (ii) a CD27 intracellular signaling domain or functional fragment thereof. In some embodiments, a CAR of the present disclosure comprises (a) a CD28 transmembrane domain, and (b) an intracellular signaling domain comprising: (i) a CD3ζ intracellular signaling domain or functional fragment thereof; and (ii) a 4-1BB intracellular signaling domain or functional fragment thereof. In some embodiments, a CAR of the present disclosure comprises (a) a CD28 transmembrane domain, and (b) an intracellular signaling domain comprising: (i) a CD3ζ intracellular signaling domain or functional fragment thereof; and (ii) an FcεCRI gamma chain intracellular signaling domain or functional fragment thereof. In some embodiments, a CD27 intracellular signaling domain or functional fragment thereof is or comprises a CD27 co-stimulatory domain. In some embodiments, a FcεRI intracellular signaling domain or functional fragment thereof is or comprises a FcεRI co-stimulatory domain. In some embodiments, a 4-1BB intracellular signaling domain or functional fragment thereof is or comprises a 4-1BB co-stimulatory domain.

The present disclosure also includes functional variants of any of CAR, or CAR domain/component, described herein. CAR functional variants encompass, for example, variants of a CAR described herein (a parent CAR) that retains the ability to recognize a particular target cell to a similar extent, the same extent, or to a higher extent, as the parent CAR. In reference to a nucleic acid sequence encoding a parent CAR, a nucleic acid sequence encoding a functional variant of the CAR can be for example, about 10% identical, about 25% identical, about 30% identical, about 50% identical, about 65% identical, about 80% identical, about 90% identical, about 95% identical, or about 99% identical to the nucleic acid sequence encoding the parent CAR. In some embodiments, a parent CAR comprises an amino acid sequence as set forth in SEQ ID NO: 10 or 13. Alternatively or additionally, in some embodiments, a CAR functional variant comprises the amino acid sequence of a parent CAR with at least one non-conservative amino acid substitution. In some embodiments, a non-conservative amino acid substitution does not compromise or inhibit a biological activity of a CAR functional variant. In some embodiments, a non-conservative amino acid substitution may enhance a biological activity of a CAR functional variant, such that biological activity of the functional variant is increased relative to its parent CAR.

The present disclosure further provides for CARs comprising an extracellular domain directed to any target molecule of interest (e.g., comprising any of known antigen-binding domain, e.g., antibody, scFv, etc.), and further comprising any transmembrane domain described herein (including any hinge domain described herein), any intracellular signaling domain described herein (including any signal sequences or motifs, any co-stimulatory domains, etc., described herein), present in any combination.

In some embodiments, a CAR comprises: (a) a hinge region, (b) a transmembrane domain derived from a human CD8α polypeptide, (c) an intracellular signaling domain comprising: (i) a human CD3ζ intracellular signaling domain or fragment thereof; and (ii) a human CD28 intracellular signaling domain or fragment thereof, wherein the CD28 intracellular signaling domain or fragment thereof is or comprises a co-stimulatory domain. In some embodiments, a CAR comprises: (a) a hinge region derived from a human CD8α polypeptide, (b) a transmembrane domain derived from a human CD8α polypeptide, (c) an intracellular signaling domain comprising: (i) a human CD3ζ intracellular signaling domain; and (ii) a human CD28 intracellular signaling domain. In some embodiments, a CAR comprises a sequence as set forth in SEQ ID NO: 27.

In some embodiments, a CAR comprises: (a) a hinge region, (b) a transmembrane domain derived from a human CD8α polypeptide, (c) an intracellular signaling domain comprising: (i) a human CD3ζ intracellular signaling domain or fragment thereof; and (ii) a CD27 and/or a CD28 intracellular signaling domain or fragment thereof, wherein the CD27 and/or CD28 intracellular signaling domain or fragment thereof is or comprises a co-stimulatory domain.

In some embodiments, a CAR comprises: (a) a hinge region, (b) a transmembrane domain derived from a human CD8α polypeptide, (c) an intracellular signaling domain comprising: (i) a human CD3ζ intracellular signaling domain or fragment thereof; and (ii) a human CD28, a human CD27, and/or an FcεRI gamma chain intracellular signaling domain or fragment thereof, wherein the human CD28, the human CD27, and/or the FcεRI gamma chain intracellular signaling domain or fragment thereof are or comprise a co-stimulatory domain.

In some embodiments, a CAR can comprises: (a) a hinge region, (b) a transmembrane domain derived from a human CD8α polypeptide, (c) an intracellular signaling domain comprising: (i) a human CD3ζ intracellular signaling domain; and (ii) a human CD28 and/or an FcεRI gamma chain intracellular signaling domain, wherein the CD28 and/or the FcεRI gamma chain intracellular signaling domain or fragment thereof are or comprise a co-stimulatory domain.

In some embodiments, a CAR as described herein, further comprises a signal peptide sequence. In some embodiments, a signal peptide is positioned at the amino terminus of an extracellular domain (e.g., at the N-terminus of an antigen-binding domain). A signal peptide as used in accordance with the present disclosure may comprise any suitable signal peptide sequence. In some embodiments, a signal peptide sequence is a human granulocyte macrophage colony-stimulating factor (GM-CSF) receptor signal peptide sequence or a CD8α signal peptide sequence. In some embodiments, a CAR provided herein comprises a human scFv comprising a CD8α signal peptide sequence. In some embodiments, a signal peptide sequence comprises an amino acid sequence as set forth in SEQ ID NO: 15.

In some embodiments, a provided CAR comprises: (a) a CD8α hinge region comprising SEQ ID NO: 28, (b) a CD8α transmembrane domain comprising SEQ ID NO: 11, (c) a CD28 intracellular signaling domain comprising SEQ ID NO: 21, and (d) a CD3ζ intracellular signaling domain comprising SEQ ID NO: 23. In some embodiments, a provided CAR comprises, from N-terminus to C-terminus: (a) a CD8α hinge region comprising SEQ ID NO: 28, (b) a CD8α transmembrane domain comprising SEQ ID NO: 11, (c) a CD28 intracellular signaling domain comprising SEQ ID NO: 21, and (d) a CD3ζ intracellular signaling domain comprising SEQ ID NO: 23.

In some embodiments, a provided CAR comprises: (a) an antigen-binding domain comprising SEQ ID NO: 17, (b) a CD8α hinge region comprising SEQ ID NO: 28, (c) a CD8α transmembrane domain comprising SEQ ID NO: 11, (d) a CD28 intracellular signaling domain comprising SEQ ID NO: 21, and (e) a CD3ζ intracellular signaling domain comprising SEQ ID NO: 23. In some embodiments, a provided CAR comprises, from N-terminus to C-terminus: (a) an antigen-binding domain comprising SEQ ID NO: 17, (b) a CD8α hinge region comprising SEQ ID NO: 28, (c) a CD8α transmembrane domain comprising SEQ ID NO: 11, (d) a CD28 intracellular signaling domain comprising SEQ ID NO: 21, and (e) a CD3ζ intracellular signaling domain comprising SEQ ID NO: 23.

In some embodiments, a provided CAR comprises: (a) a CD8α signal peptide sequence comprising SEQ ID NO: 15, (b) an antigen-binding domain comprising SEQ ID NO: 17, (c) a CD8α hinge region as set forth in SEQ ID NO: 28, (d) a CD8α transmembrane domain as set forth in SEQ ID NO: 11, (e) a CD28 intracellular signaling domain as set forth in SEQ ID NO: 21, and (f) a CD3ζ intracellular signaling domain as set forth in SEQ ID NO: 23. In some embodiments, a provided CAR comprises, from N-terminus to C-terminus: (a) a CD8α signal peptide sequence comprising SEQ ID NO: 15, (b) an antigen-binding domain comprising SEQ ID NO: 17, (c) a CD8α hinge region as set forth in SEQ ID NO: 28, (d) a CD8α transmembrane domain as set forth in SEQ ID NO: 11, (e) a CD28 intracellular signaling domain as set forth in SEQ ID NO: 21, and (f) a CD3ζ intracellular signaling domain as set forth in SEQ ID NO: 23.

In some embodiments, a CAR having any of the combinations of transmembrane domain, intracellular domain(s), and optionally hinge domain, as described above, further comprises an extracellular domain that binds CD19 (e.g., human CD19). In some embodiments, the extracellular domain comprises a means for binding CD19. Exemplary CD19-binding domains are described in the “Extracellular Domain” subsection above.

In some embodiments, a CAR of the present disclosure comprises an amino acid sequence having at least 70%, at least 75%, at least 80%, at least 85%, at least 90%, at least 95%, at least 96%, at least 97%, at least 98%, at least 99%, or greater sequence identity to SEQ ID NO: 10. In some embodiments, a CAR of the present disclosure comprises an amino acid sequence having at least 80% sequence identity to SEQ ID NO: 10. In some embodiments, a CAR of the present disclosure comprises an amino acid sequence having at least 85% sequence identity to SEQ ID NO: 10. In some embodiments, a CAR of the present disclosure comprises an amino acid sequence having at least 90% sequence identity to SEQ ID NO: 10. In some embodiments, a CAR of the present disclosure comprises an amino acid sequence having at least 95% sequence identity to SEQ ID NO: 10. In some embodiments, a CAR of the present disclosure comprises an amino acid sequence having at least 96% sequence identity to SEQ ID NO: 10. In some embodiments, a CAR of the present disclosure comprises an amino acid sequence having at least 97% sequence identity to SEQ ID NO: 10. In some embodiments, a CAR of the present disclosure comprises an amino acid sequence having at least 98% sequence identity to SEQ ID NO: 10. In some embodiments, a CAR of the present disclosure comprises an amino acid sequence having at least 99% sequence identity to SEQ ID NO: 10. In some embodiments, a CAR of the present disclosure comprises an amino acid sequence as set forth in SEQ ID NO: 10.

In some embodiments, a CAR of the present disclosure comprises an amino acid sequence having at least 70%, at least 75%, at least 80%, at least 85%, at least 90%, at least 95%, at least 96%, at least 97%, at least 98%, at least 99%, or greater sequence identity to SEQ ID NO: 13. In some embodiments, a CAR of the present disclosure comprises an amino acid sequence having at least 80% sequence identity to SEQ ID NO: 13. In some embodiments, a CAR of the present disclosure comprises an amino acid sequence having at least 85% sequence identity to SEQ ID NO: 13. In some embodiments, a CAR of the present disclosure comprises an amino acid sequence having at least 90% sequence identity to SEQ ID NO: 13. In some embodiments, a CAR of the present disclosure comprises an amino acid sequence having at least 95% sequence identity to SEQ ID NO: 13. In some embodiments, a CAR of the present disclosure comprises an amino acid sequence having at least 96% sequence identity to SEQ ID NO: 13. In some embodiments, a CAR of the present disclosure comprises an amino acid sequence having at least 97% sequence identity to SEQ ID NO: 13. In some embodiments, a CAR of the present disclosure comprises an amino acid sequence having at least 98% sequence identity to SEQ ID NO: 13. In some embodiments, a CAR of the present disclosure comprises an amino acid sequence having at least 99% sequence identity to SEQ ID NO: 13. In some embodiments, a CAR of the present disclosure comprises an amino acid sequence as set forth in SEQ ID NO: 13.

In some embodiments, a CAR of the present disclosure is encoded by nucleic acid sequence having at least 70%, at least 75%, at least 80%, at least 85%, at least 90%, at least 95%, at least 96%, at least 97%, at least 98%, at least 99%, or greater sequence identity to SEQ ID NO: 14. In some embodiments, a CAR of the present disclosure is encoded by a nucleic acid sequence having at least 80% sequence identity to SEQ ID NO: 14. In some embodiments, a CAR of the present disclosure is encoded by a nucleic acid sequence having at least 85% sequence identity to SEQ ID NO: 14. In some embodiments, a CAR of the present disclosure is encoded by a nucleic acid sequence having at least 90% sequence identity to SEQ ID NO: 14. In some embodiments, a CAR of the present disclosure is encoded by a nucleic acid sequence having at least 95% sequence identity to SEQ ID NO: 14. In some embodiments, a CAR of the present disclosure is encoded by a nucleic acid sequence having at least 96% sequence identity to SEQ ID NO: 14. In some embodiments, a CAR of the present disclosure is encoded by a nucleic acid sequence having at least 97% sequence identity to SEQ ID NO: 14. In some embodiments, a CAR of the present disclosure is encoded by a nucleic acid sequence having at least 98% sequence identity to SEQ ID NO: 14. In some embodiments, a CAR of the present disclosure is encoded by a nucleic acid sequence having at least 99% sequence identity to SEQ ID NO: 14. In some embodiments, a CAR of the present disclosure is encoded by a nucleic acid sequence as set forth in SEQ ID NO: 14.

It has been observed that T cells engineered to express a CD19-CAR that incorporates a 4-1BB costimulatory domain produced substantially higher background levels of IFNγ, in the absence CD19-expressing target cells, than T cells engineered to express a CD19-CAR that incorporates a costimulatory domain derived from CD28, CD27, or FcεRI gamma chain. Without wishing to be bound by theory, it is hypothesized that the higher background level of cytokine production is undesirable for treating autoimmune diseases. Therefore, in certain embodiments, a CAR disclosed herein does not comprise an intracellular T cell signaling domain derived from 4-1BB.

### Nucleic Acid Constructs

The present disclosure further provides for an engineered nucleic acid, or nucleic acid construct, comprising a nucleic acid sequence that encodes any polypeptide described herein, e.g., any CAR described herein. In some embodiments, an engineered nucleic acid comprises a promoter operably linked to a nucleic acid sequence that encodes a CAR (e.g., any CAR described herein). Any appropriate promoter may be operably linked to any of the engineered nucleic acid sequences described herein. Non-limiting examples of promoters that may be used in accordance with the present disclosure include EF1a, SFFV, PGK, CMV, CAG, UbC, murine stem cell virus (MSCV), MND, EF1a hybrid promoters, CAG hybrid promoters, or derivatives or functional fragments thereof. In some embodiments, a promoter is an EF1a promoter. In some embodiments, promoter is a SFFV promoter. In some embodiments, a promoter is a PGK promoter. In some embodiments, a promoter is a CMV promoter. In some embodiments, a promoter is a CAG promoter. In some embodiments, a promoter is a UbC promoter. In some embodiments, a promoter is a MSCV promoter. In some embodiments, a promoter is a MND promoter.

In some cases, an engineered nucleic acid comprises sufficient cis-acting elements (e.g., a promoter and/or an enhancer) that supplement expression of a provided engineered nucleic acid sequence where the remaining elements needed for expression can be supplied by a host cell (e.g., a mammalian cell, e.g., a T cell) or in an in vitro expression system.

The present disclosure also provides for vectors, or plasmids, comprising any engineered nucleic acid as described herein. The present disclosure provides for transposons, cosmids, viral vectors (e.g., any adenoviral vectors (e.g., pSV or pCMV vectors), adeno-associated virus (AAV) vectors, lentivirus vectors, and retroviral vectors), and any Gateway® vectors comprising any engineered nucleic acid described herein. In some embodiments, a viral vector is selected from the group consisting of: a lentiviral vector, a retroviral vector, an adenoviral vector, and an adeno-associated viral (AAV) vector. In some embodiments, a viral vector is a lentiviral vector. In some embodiments, a viral vector is a retroviral vector. In some embodiments, a viral vector is an adenoviral vector. In some embodiments, a viral vector is a AAV vector.

Exemplary lentiviral vectors that may be used in accordance with the present disclosure include vectors derived from human immunodeficiency virus-1 (HIV-1), human immunodeficiency virus-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV).

Retroviral vectors typically are constructed such that the majority of sequences coding for the structural genes of the virus are deleted and replaced by a gene of interest or expression cassette of interest (e.g., an engineered nucleic acid as described here). Most often, the structural genes (i.e., gag, pol, and env), are removed from the retroviral backbone using genetic engineering techniques known in the art. This may include digestion with the appropriate restriction endonuclease or, in some instances, with Bal 31 exonuclease to generate fragments containing appropriate portions of the packaging signal. Accordingly, in some embodiments, a minimum retroviral vector comprises from 5′ to 3′: a 5′ long terminal repeat (LTR), a packaging signal, an optional exogenous promoter and/or enhancer, an exogenous gene of interest (or engineered nucleic acid), and a 3′ LTR. In some embodiments, if no exogenous promoter is provided, gene expression may be driven by the 5′ LTR, which is a weak promoter and requires the presence of Tat to activate expression. In many embodiments, structural genes can be provided in separate vectors for manufacture of the lentivirus, rendering the produced virions replication-defective. Specifically, with respect to lentivirus, the packaging system may comprise a single packaging vector encoding the Gag, Pol, Rev, and Tat genes, and a third, separate vector encoding the envelope protein Env (usually VSV-G due to its wide infectivity). To improve the safety of the packaging system, the packaging vector can be split, expressing Rev from one vector, Gag and Pol from another vector. Tat can also be eliminated from the packaging system by using a retroviral vector comprising a chimeric 5′ LTR, wherein the U3 region of the 5′ LTR is replaced with a heterologous regulatory element.

Nucleic acids (e.g., genes) to be packaged into a retrovirus (e.g., a lentivirus) can be incorporated into the proviral backbone in several general ways. The most straightforward constructions are ones in which the structural genes of the retrovirus are replaced by a single gene which then is transcribed under the control of the viral regulatory sequences within the LTR. Retroviral vectors have also been constructed which can introduce more than one gene into target cells. Usually, in such vectors one gene is under the regulatory control of the viral LTR, while the second gene is expressed either off a spliced message or is under the regulation of its own, internal promoter.

Accordingly, nucleic acids (e.g., genes) to be packaged into a retrovirus are flanked by 5′ and 3′ LTRs, which serve to promote transcription and polyadenylation of the virion RNAs, respectively. The term “long terminal repeat” or “LTR” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals, and sequences needed for replication and integration of the viral genome. The U3 region contains the enhancer and promoter elements. The U5 region is the sequence between the primer binding site and the R region and contains the polyadenylation sequence. The R (repeat) region is flanked by the U3 and U5 regions. In certain embodiments, the R region comprises a trans-activation response (TAR) genetic element, which interacts with the trans-activator (tat) genetic element to enhance viral replication. This element is not required in embodiments wherein the U3 region of the 5′ LTR is replaced by a heterologous promoter.

In some embodiments, a retroviral vector comprises a modified 5′ LTR and/or 3′ LTR. Modifications of the 3′ LTR are often made to improve the safety of lentiviral or retroviral systems by rendering viruses replication-defective. In some embodiments, a retroviral vector is a self-inactivating (SIN) vector. As used herein, a SIN retroviral vector refers to a replication-defective retroviral vector in which the 3′ LTR U3 region has been modified (e.g., by deletion or substitution) to prevent viral transcription beyond the first round of viral replication. This is because the 3′ LTR U3 region is used as a template for the 5′ LTR U3 region during viral replication and, thus, the viral transcript cannot be made without the U3 enhancer-promoter. In some embodiments, a 3′ LTR is modified such that the U5 region is replaced, for example, with an ideal polyadenylation sequence. It should be noted that modifications to the LTRs such as modifications to the 3′ LTR, the 5′ LTR, or both 3′ and 5′ LTRs, are also included in some embodiments of the present disclosure.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus, because there is no complete U3 sequence in the virus production system.

Adjacent to a 5′ LTR are sequences necessary for reverse transcription of the genome (the tRNA primer binding site) and for efficient packaging of viral RNA into particles (the Psi site). As used herein, the term “packaging signal” or “packaging sequence” refers to sequences located within the retroviral genome which are required for encapsidation of retroviral RNA strands during viral particle formation (see e.g., Clever et al., 1995 J. Virology, 69(4):2101-09). The packaging signal may be a minimal packaging signal (also referred to as the psi [Ψ] sequence) needed for encapsidation of the viral genome.

In some embodiments, a retroviral vector (e.g., lentiviral vector) further comprises a FLAP. As used herein, the term “FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou et al. (2000) Cell 101:173. During reverse transcription, central initiation of the plus-strand DNA at the cPPT and central termination at the CTS lead to the formation of a three-stranded DNA structure: a central DNA flap. While not wishing to be bound by any theory, the DNA flap may act as a cis-active determinant of lentiviral genome nuclear import and/or may increase the titer of the virus. In some embodiments, retroviral vector backbones comprise one or more FLAP elements upstream or downstream of the heterologous genes of interest in the vectors. For example, in some embodiments, a transfer plasmid includes a FLAP element. In some embodiments, a vector of the present disclosure comprises a FLAP element isolated from HIV-1.

In some embodiments, a retroviral vector (e.g., lentiviral vector) further comprises an export element. In some embodiments, retroviral vectors comprise one or more export elements. The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) RRE (see e.g., Cullen et al., (1991) J. Virol. 65: 1053; and Cullen et al., (1991) Cell 58: 423) and the hepatitis B virus post-transcriptional regulatory element (HPRE). Generally, the RNA export element is placed within the 3′ UTR of a gene, and can be inserted as one or multiple copies.

In some embodiments, a retroviral vector (e.g., lentiviral vector) further comprises a posttranscriptional regulatory element. A variety of posttranscriptional regulatory elements can increase expression of a heterologous nucleic acid, e.g., woodchuck hepatitis virus posttranscriptional regulatory element (WPRE; see Zufferey et al., (1999) J. Virol., 73:2886); the posttranscriptional regulatory element present in hepatitis B virus (HPRE) (Huang et al., Mol. Cell. Biol., 5:3864); an optimized posttranscriptional regulatory element (oPRE; see Schambach et al., (2006) Gene Therapy 13, 641-45); and the like (Liu et al., (1995), Genes Dev., 9:1766). The posttranscriptional regulatory element is generally positioned at the 3′ end the heterologous nucleic acid sequence. This configuration results in synthesis of an mRNA transcript whose 5′ portion comprises the heterologous nucleic acid coding sequences and whose 3′ portion comprises the posttranscriptional regulatory element sequence. In some embodiments, vectors of the present disclosure lack or do not comprise a posttranscriptional regulatory element such as a WPRE or HPRE, because in some instances these elements increase the risk of cellular transformation and/or do not substantially or significantly increase the amount of mRNA transcript or increase mRNA stability. Therefore, in certain embodiments, vectors of the present disclosure lack or do not comprise a WPRE or HPRE as an added safety measure.

Elements directing the efficient termination and polyadenylation of the heterologous nucleic acid transcripts increase heterologous gene expression. Transcription termination signals are generally found downstream of the polyadenylation signal. Accordingly, in some embodiments, a retroviral vector (e.g., lentiviral vector) further comprises a polyadenylation signal. The term “polyadenylation signal” or “polyadenylation sequence” as used herein denotes a DNA sequence which directs both the termination and polyadenylation of the nascent RNA transcript by RNA polymerase H. Efficient polyadenylation of the recombinant transcript is desirable as transcripts lacking a polyadenylation signal are unstable and are rapidly degraded. Illustrative examples of polyadenylation signals that can be used in a vector of the present disclosure, include an ideal polyadenylation sequence (e.g., AATAAA, ATTAAA AGTAAA), a bovine growth hormone polyadenylation sequence (BGHpA), a rabbit P-globin polyadenylation sequence (rpgpA), or another suitable heterologous or endogenous polyadenylation sequence known in the art.

In some embodiments, a retroviral vector further comprises an insulator element. Insulator elements may contribute to protecting retrovirus-expressed sequences, e.g., therapeutic genes, from integration site effects, which may be mediated by cis-acting elements present in genomic DNA and lead to deregulated expression of transferred sequences (i.e., position effect; see, e.g., Burgess-Beusse et al., (2002) Proc. Natl. Acad. Sci., USA, 99:16433; and Zhan et al., 2001, Hum. Genet., 109:471). In some embodiments, a retroviral vector comprises an insulator element in one or both LTRs or elsewhere in the region of the vector that integrates into the cellular genome. Suitable insulators for use in the present disclosure include, but are not limited to, the chicken β-globin insulator (see Chung et al., (1993). Cell 74:505; Chung et al., (1997) Proc. Natl. Acad. Sci., USA 94:575; and Bell et al., 1999. Cell 98:387). Examples of insulator elements include, but are not limited to, an insulator from a β-globin locus, such as chicken HS4.

Non-limiting examples of lentiviral vectors include pLVX-EF1alpha-AcGFP1-C1 (Clontech Catalog #631984), pLVX-EF1alpha-IRES-mCherry (Clontech Catalog #631987), pLVX-Puro (Clontech Catalog #632159), pLVX-IRES-Puro (Clontech Catalog #632186), pLenti6/V5-DEST™ (Thermo Fisher), pLenti6.2/V5-DEST™ (Thermo Fisher), pLKO.1 (Plasmid #10878 at Addgene), pLKO.3G (Plasmid #14748 at Addgene), pSico (Plasmid #11578 at Addgene), pLJM1-EGFP (Plasmid #19319 at Addgene), FUGW (Plasmid #14883 at Addgene), pLVTHM (Plasmid #12247 at Addgene), pLVUT-tTR-KRAB (Plasmid #11651 at Addgene), pLL3.7 (Plasmid #11795 at Addgene), pLB (Plasmid #11619 at Addgene), pWPXL (Plasmid #12257 at Addgene), pWPI (Plasmid #12254 at Addgene), EF.CMV.RFP (Plasmid #17619 at Addgene), pLenti CMV Puro DEST (Plasmid #17452 at Addgene), pLenti-puro (Plasmid #39481 at Addgene), pULTRA (Plasmid #24129 at Addgene), pLX301 (Plasmid #25895 at Addgene), pHIV-EGFP (Plasmid #21373 at Addgene), pLV-mCherry (Plasmid #36084 at Addgene), pLionII (Plasmid #1730 at Addgene), plnducer10-mir-RUP-PheS (Plasmid #44011 at Addgene). These vectors can be modified to be suitable for therapeutic use. For example, a selection marker (e.g., puro, EGFP, or mCherry) can be deleted or replaced with a second exogenous gene of interest. Further examples of lentiviral vectors are disclosed in U.S. Pat. Nos. 7,629,153, 7,198,950, 8,329,462, 6,863,884, 6,682,907, 7,745,179, 7,250,299, 5,994,136, 6,287,814, 6,013,516, 6,797,512, 6,544,771, 5,834,256, 6,958,226, 6,207,455, 6,531,123, and 6,352,694, and PCT Publication No. WO2017/091786.

In some embodiments, an engineered nucleic acid of the present disclosure comprises a nucleic acid sequence encoding a CAR, wherein the nucleic acid sequence has at least 70%, at least 75%, at least 80%, at least 85%, at least 90%, at least 95%, at least 96%, at least 97%, at least 98%, at least 99%, or greater sequence identity to SEQ ID NO: 14. In some embodiments, an engineered nucleic acid of the present disclosure comprises a nucleic acid sequence having at least 80% sequence identity to SEQ ID NO: 14. In some embodiments, an engineered nucleic acid of the present disclosure comprises a nucleic acid sequence having at least 85% sequence identity to SEQ ID NO: 14. In some embodiments, an engineered nucleic acid of the present disclosure comprises a nucleic acid sequence having at least 90% sequence identity to SEQ ID NO: 14. In some embodiments, an engineered nucleic acid of the present disclosure comprises a nucleic acid sequence having at least 95% sequence identity to SEQ ID NO: 14. In some embodiments, an engineered nucleic acid of the present disclosure comprises a nucleic acid sequence having at least 96% sequence identity to SEQ ID NO: 14. In some embodiments, an engineered nucleic acid of the present disclosure comprises a nucleic acid sequence having at least 97% sequence identity to SEQ ID NO: 14. In some embodiments, an engineered nucleic acid of the present disclosure comprises a nucleic acid sequence having at least 98% sequence identity to SEQ ID NO: 14. In some embodiments, an engineered nucleic acid of the present disclosure comprises a nucleic acid sequence having at least 99% sequence identity to SEQ ID NO: 14. In some embodiments, an engineered nucleic acid of the present disclosure comprises a nucleic acid sequence as set forth in SEQ ID NO: 14.

In certain embodiments, a lentivirus vector disclosed herein comprises a truncated 5′ LTR (e.g., with deletion of its U3 region), an HIV-1Ψ packaging sequence, a MSCV promoter operably linked to a nucleic acid encoding a CAR (e.g., any of the CARs as disclosed herein), and a truncated 3′ LTR (e.g., with deletion of its U3 region). In certain embodiments, the lentivirus vector further comprises a RRE, a cPPT/CTS, and/or an oPRE. In certain embodiments, the lentivirus vector comprises a truncated 5′ LTR (e.g., with deletion of its U3 region), an HIV-1Ψ packaging sequence, a RRE, a cPPT/CTS, a MSCV promoter operably linked to a nucleic acid encoding a CAR (e.g., any of the CARs as disclosed herein), an oPRE, and a truncated 3′ LTR (e.g., with deletion of its U3 region). In certain embodiments, the lentivirus vector is pseudotyped with VSV-G envelope protein.

### Methods of Making Engineered T Cells

Also provided herein are methods of making an engineered T cell, the methods comprising introducing into a host T cell an engineered nucleic acid comprising a nucleic acid sequence encoding a CAR (e.g., any CAR described herein). Accordingly in some embodiments, an engineered T cell refers to a genetically modified T cell that has been modified to express a CAR, e.g., any provided anti-CD19 CAR.

In some embodiments, a host T cell used to make an engineered T cell can be any T cell such as a cultured T cell, e.g., a primary T cell, or a T cell derived from a cultured T cell line, e.g., a Jurkat, SupT1, etc., or a T cell obtained from a mammal. In some embodiments, a T cell used to make an engineered T cell can be selected from naive T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. In some embodiments, a host T cell used to make an engineered T cell can be a CD3+ cell. In some embodiments, a host T cell can be CD4+, CD8+, or CD4+ and CD8+. In some embodiments, a host T cell can be any type of T cell, e.g., CD4+/CD8+ double positive T cells, CD4+ helper T cells (e.g., Th1 and Th2 cells), CD8+ T cells (e.g., cytotoxic T cells), memory T cells, naive T cells, regulatory T cells, T6T cells, etc. In some embodiments, a host T cell used to make an engineered T cell can be any T cell at any stage of development. Additional types of helper T cells include Th3 (Treg) cells, Th17 cells, Th9 cells, or Tfh cells. Additional types of memory T cells include cells such as central memory T cells (Tcm cells), effector memory T cells (Tem cells and TEMRA cells). In some embodiments, obtained host T cells are substantially free of non-T cells.

The T cells can be obtained from various biological samples of a subject (e.g., a human subject). Non-limiting examples of biological sample include cells, tissue (e.g., tissue obtained by biopsy), blood, serum, plasma, or any sample derived therefrom. In certain embodiments, the sample is a whole blood sample or an apheresis (e.g., leukapheresis) sample obtained from the subject. In certain embodiments, the method comprises obtaining the sample from the subject. In certain embodiments, the method comprises having obtained the sample from the subject.

In certain embodiments, the T cells are isolated from the sample. Isolation of T cells may include an initial purification of T cells from a mixture of plasma, lymphocytes, platelets, red blood cells, monocytes, and granulocytes. Methods for isolation of T cells from a biological sample, such as a whole blood sample or a leukapheresis sample, are well-known. Exemplary methods may include leukapheresis, elutriation, density gradient centrifugation, enrichment by selection, and the like. For example, the method may include obtaining or having obtained a biological sample, such as a fresh, refrigerated, frozen, or cryopreserved leukapheresis product or alternative source of hematopoietic tissue, such as a whole blood sample, bone marrow sample, or a tumor or organ biopsy or removal (e.g., thymectomy) from an entity, such as a laboratory, hospital, or healthcare provider, and performing the aforementioned isolation steps to produce an enriched population of T cells (e.g., starting population of T cells) suitable for expression of a heterologous protein.

Furthermore, the purity of the T cell population can be increased by using one or more selection steps, such as negative selection or positive selection. Negative selection typically involves removal of undesired cell types from a mixed population of cells in a sample using one or more agents that selectively bind to the undesired cell type, whereas positive selection typically involves isolation of the desired cell population using one or more agents that selectively bind to the desired cell type. Enrichment of a T cell population by negative selection can be accomplished, for example, with a combination of antibodies directed to surface markers unique to the negatively selected cells. One method is cell sorting and/or selection via negative magnetic immuno-adherence or flow cytometry that uses a cocktail of monoclonal antibodies directed to cell surface markers present on the negatively selected cells. For example, to enrich for CD4+ cells by negative selection, a monoclonal antibody cocktail can include antibodies to CD14, CD20, CDb, CD16, HLA-DR, and CD8. On the other hand, a positive selection step can be used to specifically select for the desired cell type. Positive selection of T cells can, in certain embodiments, include incubation of a mixed population of cells that contains the T cells with a CD3-binding agent (e.g., anti-CD3 antibody-conjugated beads) for a time sufficient for positive selection of the desired T cells.

In some embodiments, engineered T cells are made using a mixture of cells (e.g., a mixture of host cells). For example, a mixture of cells may be obtained (e.g., from a subject), and an engineered nucleic acid may be inserted into the mixture of cells such that a mixture of engineered cells is made. In some embodiments, a mixture of cells comprises a mixture of T cells (e.g., any T cells described herein). In some embodiments, a mixture of cells comprises CD4+ and/or CD8+ T cells. In some embodiments, a mixture of cells comprises CD4+ and CD8+ T cells. In some embodiments, a mixture of cells is obtained by enriching for CD4+ and CD8+ T cells, yielding an enriched mixture of CD4+ and CD8+ cells. In certain embodiments, the mixture of cells comprises 1-10%, 1-20%, 1-30%, 1-40%, 1-50%, 1-60%, 10-20%, 10-30%, 10-40%, 10-50%, 10-60%, 20-30%, 20-40%, 20-50%, 20-60%, 30-40%, 30-50%, or 30-60% of CD8+ T cells (e.g., CD8+ cytotoxic T cells) out of all T cells in the population. In certain embodiments, the mixture of cells further comprises 1-10%, 1-20%, 1-30%, 1-40%, 1-50%, 1-60%, 1-70%, 10-20%, 10-30%, 10-40%, 10-50%, 10-60%, 10-70%, 20-30%, 20-40%, 20-50%, 20-60%, 20-70%, 30-40%, 30-50%, 30-60%, or 30-70% of CD4+ T cells (e.g., CD4+ helper T cells) out of all T cells in the population. In certain embodiments, the mixture of cells comprise CD8+ T cells (e.g., CD8+ cytotoxic T cells) and CD4+ T cells (e.g., CD4+ helper T cells) at a ratio of 1:5 to 5:1, 1:4 to 4:1, 1:3 to 3:1, 1:2 to 2:1, 1:5 to 2:1, 1:4 to 2:1, 1:3 to 1:1, or 1:2 to 1:1. In some embodiments, the mixture of cells comprise CD8+ T cells and CD4+ T cells at a ratio of about 1:2. In some embodiments, a host cell or mixture of host cells are expanded before introduction of an engineered nucleic acid or vector or plasmid comprising an engineered nucleic acid. In some embodiments, a host cell or mixture of host cells are allogeneic. In some embodiments, a host cell or mixture of host cells are autologous.

In some embodiments, introducing an engineered nucleic acid (or a vector or plasmid comprising an engineered nucleic acid) to a host cell comprises contacting the host cell with a viral vector (e.g., any viral vector described herein). In some embodiments, a viral vector is selected from the group consisting of: a lentiviral vector, a retroviral vector, an adenoviral vector, transposons, cosmids, and an AAV vector. In some embodiments, a viral vector is a lentiviral vector. In some embodiments, a step of introducing an engineered nucleic acid (or a vector or plasmid comprising an engineered nucleic acid) to a host cell comprises use of viral transduction. Any known method of introducing nucleic acids (including nucleic acid vectors and plasmids) into a host cell may be used in accordance with the present disclosure.

Methods of introducing nucleic acid constructs into a cell (e.g., a eukaryotic cell) are known in the art. Non-limiting examples of methods that can be used to introduce an engineered nucleic acid or nucleic acid construct (e.g., a vector or plasmid comprising an engineered nucleic acid) into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral and lentiviral transduction), and nanoparticle transfection. As used herein, “transformed” and “transduced” are used interchangeably.

In some embodiments, an engineered nucleic acid is introduced to a cell using a lentiviral vector. In some embodiments, the lentiviral vector is used at a multiplicity of infection (MOI) of about 1, about 2, about 3, about 4, about 5, about 6, about 7, about 8, about 9, about 10, or greater. In some embodiments, the lentiviral vector is used at an MOI of about 1. In some embodiments, the lentiviral vector is used at an MOI of about 2. In some embodiments, the lentiviral vector is used at an MOI of about 3. In some embodiments, the lentiviral vector is used at an MOI of about 4. In some embodiments, the lentiviral vector is used at an MOI of about 5. In some embodiments, the lentiviral vector is used at an MOI of about 6. In some embodiments, the lentiviral vector is used at an MOI of about 7. In some embodiments, the lentiviral vector is used at an MOI of about 8. In some embodiments, the lentiviral vector is used at an MOI of about 9. In some embodiments, the lentiviral vector is used at an MOI of about 10.

In some embodiments, a provided method further includes a step of contacting a host T cell with an effective amount of one or more CD3-stimulation agents in the absence of a CD28 stimulating agent under conditions that allow for the stimulation of the host T cell. In other embodiments, a provided method further includes a step of contacting a host T cell with an effective amount of one or more agents that activate both CD3 and CD28 (e.g., a solid surface, such as a polymeric nanomatrix, coated with an anti-CD3 antibody and an anti-CD28 antibody) under conditions that allow for the stimulation of the host T cell.

In some embodiments, the present disclosure provides a method of making an engineered T cell, the method comprising steps of: (a) obtaining a host T cell from a subject; and (b) introducing an engineered nucleic acid (e.g., any engineered nucleic acid described herein) to the host T cell. In some embodiments, a method of making an engineered T cell further comprises a step of contacting a host T cell with an effective amount of one or more CD3-stimulation agents in the absence of a CD28 stimulating agent under conditions that allow for the stimulation of the host T cell. In other embodiments, a method of making an engineered T cell further comprises a step of contacting a host T cell with an effective amount of one or more agents that activate both CD3 and CD28 (e.g., a magnetic bead coated with an anti-CD3 antibody and an anti-CD28 antibody) under conditions that allow for the stimulation of the host T cell. In certain embodiments, the stimulation step is taken prior to step (b).

In some embodiments, the present disclosure provides a method of making an engineered T cell, the method comprising steps of: (a) obtaining a host T cell from a subject; and (b) introducing an engineered nucleic acid (e.g., any engineered nucleic acid described herein, e.g., a vector comprising an engineered nucleic acid, using any method of introducing provided herein) to the host T cell.

In some embodiments, the present disclosure provides a method of making an engineered T cell, the method comprising steps of: (a) obtaining a host T cell mixture from a subject; and (b) introducing an engineered nucleic acid (e.g., any engineered nucleic acid described herein, e.g., a vector comprising an engineered nucleic acid, using any method of introducing provided herein) to the host T cell mixture. In some embodiments, a method of making an engineered T cell further comprises a step of enriching a host T cell mixture for CD4+ and CD8+ T cells prior to introducing an engineered nucleic acid to the host T cell mixture.

In some embodiments, a method of making an engineered T cell further comprises a step of expanding a host T cell or T cell mixture before introduction of an engineered nucleic acid or vector or plasmid comprising an engineered nucleic acid. In certain embodiments, the T cells are expanded after introduction of the engineered nucleic acid, while the nucleic acid is still in the cell culture medium. In certain embodiments, the T cells are expanded for at least 3, 4, 5, 6, 7, or 8 days in the presence of one or more cytokines, including but not limited to IL-2, IL-7, and/or IL-15. In certain embodiments, the T cells are expanded for at least 3, 4, 5, 6, 7, or 8 days in the presence of IL-7 and IL-15.

Also provided herein are engineered T cells produced using any of the methods described herein. The present disclosure provides for engineered T cells comprising an engineered nucleic acid (e.g., any of the engineered nucleic acid described herein). In many embodiments of the present disclosure an engineered T cell comprises an engineered nucleic acid encoding a CAR (e.g., any CAR described herein). In some embodiments, the present disclosure provides an engineered T cell comprising a nucleic acid sequence encoding an amino acid sequence as set forth in SEQ ID NO: 13. In some embodiments, the present disclosure provides an engineered T cell comprising an engineered nucleic acid sequence as set forth in SEQ ID NO: 14.

The population of engineered T cells produced may include T cells of various phenotypes, such as naive T (T_{N}) cells characterized as CD45RO−, CCR7+, and CD95−, central memory T (T_{CM}) cells characterized as CD45RO+ and CCR7+, effector memory T (T_{EM}) cells characterized as CD45RO+ and CCR7−, stem memory T (T_{SCM}) cells characterized as CD45RO−, CCR7+, and CD95+, and effector memory cells re-expressing CD45RA T (T_{EMRA}) cells characterized as CD45RO− and CCR7−. Alternative characterizations of these T cell subsets include but are not limited to naive T (T_{N}) cells characterized as CD45RA+, CCR7+, and CD95−, central memory T (T_{CM}) cells characterized as CD45RA- and CCR7+, effector memory T (T_{EM}) cells characterized as CD45RA- and CCR7−, stem memory T (T_{SCM}) cells characterized as CD45RA+, CCR7+, and CD95+, and effector memory cells re-expressing CD45RA T (T_{EMRA}) cells characterized as CD45RA+ and CCR7−. In some embodiments, the population comprises at least 25%, 30%, 40%, 50%, 60%, 70%, 75%, or 80% CD4+ T cells, out of all T cells in the population. In some embodiments, the population comprises at least 20%, 25%, 30%, 40%, 50%, 55%, or 60% CD8+ T cells, out of all T cells in the population. In some embodiments, the population comprises at least 0.05%, 0.1%, 0.2%, 0.3%, 0.4%, 0.5%, 1%, 2%, 3%, 4%, 5%, 6%, or 7% CD4+ T_{N}, out of all T cells in the population. In some embodiments, the population comprises at least 0.05%, 0.1%, 0.2%, 0.3%, 0.4%, 0.5%, 0.6%, 0.7%, 0.8%, 0.9%, 1%, 5%, 10%, 15%, 20%, 25%, 30%, or 35% CD4+ T_{SCM}, out of all T cells in the population. In some embodiments, the population comprises at least 10%, 15%, 20%, 25%, or 30% CD4+ TCM, out of all T cells in the population. In some embodiments, the population comprises at least 20%, 25%, 30%, 35%, 40%, 45%, 50%, 55%, 60%, 65%, 70%, 75%, 80%, or 85% CD4+ T_{EM}, out of all T cells in the population. In some embodiments, the population comprises at least 5%, 10%, 15%, 20%, 25%, or 30% CD4+ T_{EMRA}, out of all T cells in the population. In some embodiments, the population comprises at least 0.05%, 0.1%, 0.2%, 0.3%, 0.4%, 0.5%, 1%, 5%, 10%, 15%, 20%, or 25% CD8+ T_{N}, out of all T cells in the population. In some embodiments, the population comprises at least 0.05%, 0.1%, 0.2%, 0.3%, 0.4%, 0.5%, 0.6%, 0.7%, 0.8%, 0.9%, 1%, 5%, 10%, 15%, 20%, 25%, 30%, 35%, 40%, 45%, 50%, 55%, or 60% CD8+ T_{SCM}, out of all T cells in the population. In some embodiments, the population comprises at least 0.05%, 0.1%, 0.2%, 0.3%, 0.4%, 0.5%, 0.6%, 0.7%, 0.8%, 0.9%, 1%, 5%, 10%, 15%, 20%, 25%, or 30% CD8+ T_{CM}, out of all T cells in the population. In some embodiments, the population comprises at least 5%, 10%, 15%, 20%, 25%, 30%, 35%, 40%, 45%, or 50% CD8+ T_{EM}, out of all T cells in the population. In some embodiments, the population comprises at least 5%, 6%, 7%, 8%, 9%, 10%, 15%, 20%, 25%, 30%, 35%, 40%, 45%, 50%, 55%, or 60% CD8+ T_{TEMRA}, out of all T cells in the population. It will be understood by a skilled artisan that the percentage of certain T cells present in a population of engineered T cells may vary from patient to patient.

### Compositions

Also provided herein are compositions (e.g., pharmaceutical compositions) comprising any engineered T cell described herein or any engineered nucleic acid described herein. In some embodiments, a provided pharmaceutical compositions can be formulated for intravenous administration. In some embodiments, a pharmaceutical compositions can include a pharmaceutically acceptable carrier (e.g., phosphate buffered saline).

In some embodiments, a provided composition (e.g., pharmaceutical composition) is formulated in a chemically defined freezing medium. In some embodiments, the chemically defined freezing medium comprises about 1% human serum albumin and about 5% dimethyl sulfoxide. In some embodiments, the chemically defined freezing medium comprises about 50% Plasma-Lyte A™ containing about 2% human serum albumin (for final human serum albumin concentration of about 1%) and about 50% CryoStor10 containing dimethyl sulfoxide to a final concentration of about 5%.

### Kits

Also provided herein are kits that include any of the compositions described herein. For example, a kit can include one or more of any of the nucleic acid constructs described herein. In other examples, a kit can include any of engineered T cell described herein or one or more doses of a composition including any engineered T cell described herein. In some embodiments, a kit can include instructions for performing any of the methods described herein.

### Methods of Treatment

### Myasthenia Gravis

Provided herein are methods and compositions for reducing the number of B cells in a subject having myasthenia gravis. In some embodiments, the present disclosure provides a method of reducing the number of B cells producing autoantibodies that mediate autoimmunity in a tissue (e.g., muscle or neuromuscular junction) in a subject having myasthenia gravis, the method comprising administering a therapeutically effective amount of any engineered T cell described herein to the subject. Also provided herein are methods of treating a subject having myasthenia gravis, the method comprising administering a therapeutically effective amount of an engineered T cell (e.g., any engineered T cell described herein, e.g., an anti-CD19 CAR T cell) to the subject.

In some embodiments, the myasthenia gravis is Class I, Class II (e.g., Class IIa or IIb), Class III (e.g., Class IIIa or IIIb), Class IV (e.g., Class IVa or IVb), or Class V myasthenia gravis. In some embodiments, the myasthenia gravis is Class I myasthenia gravis. In some embodiments, the myasthenia gravis is Class II (e.g., Class IIa or IIb) myasthenia gravis. In some embodiments, the myasthenia gravis is Class III (e.g., Class IIIa or IIIb) myasthenia gravis. In some embodiments, the myasthenia gravis is Class IV (e.g., Class IVa or IVb) myasthenia gravis. In some embodiments, the myasthenia gravis is Class V myasthenia gravis. In some embodiments, the myasthenia gravis is an anti-acetylcholine receptor (anti-AChR) positive myasthenia gravis. In some embodiments, the myasthenia 10 gravis is an anti-AChR negative myasthenia gravis. In some embodiments, the myasthenia gravis is a refractory myasthenia gravis. In some embodiments, the myasthenia gravis is generalized myasthenia gravis (e.g., anti-AchR positive generalized myasthenia gravis). In some embodiments, the myasthenia gravis is characterized, at least in part, by deposition of complement C5b-9 complex.

In some embodiments, the subject receiving a provided treatment has an MG-Activity of Daily Living (MG-ADL) total score of 6, 7, 8, 9, 10, or greater. In some embodiments, the subject has an MG-ADL total score of greater than or equal to 6. In some embodiments, the subject has a score of greater than or equal to 2 for at least one or more sub-components for talking, chewing, swallowing, or breathing.

In some embodiments, a subject receiving a provided treatment (e.g., any engineered nucleic acid, engineered T cell, or CAR provided herein) has previously been treated with a lymphodepletion agent (e.g., cyclophosphamide and/or fludarabine). In some embodiments, a subject receiving a provided treatment has previously received a standard of care or approved treatment (e.g., any standard of care or approved treatment described herein) for myasthenia gravis that was ineffective and/or caused one or more adverse side effects. For example, in some embodiments, a subject receiving a presently provided treatment has previously been treated with an acetylcholinesterase inhibitor. In some embodiments, a subject receiving a presently provided treatment has previously been treated with an acetylcholinesterase inhibitor at a stable dose for at least 2 weeks prior to receiving the provided treatment. In some embodiments, a subject receiving a presently provided treatment has previously been treated with a B-cell depleting antibody (e.g., rituximab). In some embodiments, a subject receiving a presently provided treatment has previously been treated with a proteasome inhibitor (e.g., bortezomib). In some embodiments, a subject receiving a presently provided treatment has previously been treated with an immunosuppressive drug (e.g., azathioprine, mycophenolate mofetil (MMF), etc.). In some embodiments, a subject receiving a presently provided treatment has previously been treated with an immunosuppressive drug, such as azathioprine, at a stable dose for at least 2 months prior to receiving the provided treatment. In some embodiments, a subject receiving a presently provided treatment has previously been treated with a corticosteroid (e.g., a glucocorticoid). In some embodiments, a subject receiving a presently provided treatment has previously been treated with a corticosteroid, such as a glucocorticoid, at a stable dose for at least 1 month prior to receiving the provided treatment. In some embodiments, a subject receiving a presently provided treatment has previously been treated with non-steroidal immunosuppressants. In some embodiments, a subject receiving a presently provided treatment has previously been treated with intravenous immunoglobulin therapy. In some embodiments, a subject receiving a presently provided treatment has not been treated with intravenous immunoglobulin therapy for at least 4 weeks prior to receiving the provided treatment. In some embodiments, a subject receiving a presently provided treatment has previously been treated with therapeutic plasma exchange (PLEX). In some embodiments, a subject receiving a presently provided treatment has not been treated with therapeutic plasma exchange (PLEX) for at least 4 weeks prior to receiving the provided treatment. In some embodiments, a subject receiving a presently provided treatment has previously been treated with complement inhibitor (e.g., eculizumab, ravalizumab, zilucoplan, etc.). In some embodiments, a subject receiving a presently provided treatment has previously been treated with an FcRn inhibitor (e.g., efgartigimod, rozanolixizumab, etc.). In some embodiments, a subject receiving a presently provided treatment has previously been treated with an anti-CD38 antibody (e.g., daratumumab). In some embodiments, a subject receiving a presently provided treatment has previously undergone thymectomy. In some embodiments, a subject receiving a provided treatment has previously received a standard of care treatment (e.g., any SOC treatment described herein) and/or one or more (e.g., two or more, three or more, etc.) immunomodulatory therapies, such as immunosuppressive therapies. In some embodiments, a subject receiving a provided treatment has previously received a standard of care treatment (e.g., any SOC treatment described herein) and/or one or more (e.g., two or more, three or more, etc.) immunomodulatory therapies, such as immunosuppressive therapies, and required a further treatment to control symptoms, such as plasmapheresis or intravenous immunoglobulin therapy. In some embodiments, as subject receiving a presently provided treatment has previously been treated with repetitive plasmapheresis. In some embodiments, a subject receiving a presently provided treatment has previously undergone thymectomy. In some embodiments, a subject has previously received a treatment for myasthenia gravis (e.g., any standard treatment, or treatment regimen, e.g., those described herein) that was ineffective and/or caused one or more adverse side effects for about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, or longer before receiving a provided treatment. In some embodiments, a subject has previously received a treatment for myasthenia gravis (e.g., any standard treatment, or treatment regimen, e.g., those described herein) that was ineffective and/or caused one or more adverse side effects for about 1 year prior to receiving a provided treatment. In some embodiments, a subject has previously received a treatment for myasthenia gravis that was ineffective and/or caused one or more adverse side effects for over 1 year prior to receiving a provided treatment. In some embodiments, a subject has previously received two or more immunosuppressive/immunomodulatory therapies (e.g., any of those described herein) that were ineffective and/or caused one or more adverse side effects for over 1 year prior to receiving a provided therapy. In some embodiments, a subject has previously received at least one immunosuppressive/immunomodulatory therapy (e.g., any of those described herein) and required chronic plasmapheresis or IVIg to control symptoms and the treatments were ineffective and/or caused one or more adverse side effects for over 1 year prior to receiving a provided therapy.

In some embodiments, a subject receiving a presently provided treatment has previously received a standard of care or approved treatment for myasthenia gravis (e.g., an immunosuppressive drug), and the standard of care or approved treatment dose is reduced (e.g., by tapering) or the standard of care or approved treatment is terminated (e.g., by washing out) before T cells are obtained from the subject for making engineered T cells (e.g., via any method described herein, such as apheresis, or any common technique). In some embodiments, a standard of care or approved treatment dose is reduced or the standard of care treatment is terminated at least 1 week, at least 2 weeks, at least 3 weeks, at least 4 weeks, at least 5 weeks, at least 6 weeks, at least 7 weeks, or at least 8 weeks before T cells are obtained from the subject for making engineered T cells (e.g., by apheresis). In some embodiments, a standard of care or approved treatment dose is reduced or the standard of care or approved treatment is terminated at least 1 week, at least 2 weeks, at least 3 weeks, at least 4 weeks, at least 5 weeks, at least 6 weeks, at least 7 weeks, or at least 8 weeks prior to a subject receiving a provided treatment. In some embodiments, a standard of care or approved treatment dose is reduced or the standard of care or approved treatment is terminated at least 6 weeks prior to a subject receiving a provided treatment. In some embodiments, an anti-CD20 antibody (e.g., rituximab) treatment is terminated at least 3 months before T cells are obtained from the subject for making engineered T cells. In some embodiments, an anti-CD20 antibody (e.g., rituximab) treatment is terminated at least 3 months prior to a subject receiving a provided treatment. In some embodiments, a corticosteroid (e.g., a glucocorticoid) treatment dose is reduced to lower than or equal to 10 mg per day, e.g., of prednisone (or equivalent), within 3 days before T cells are obtained from the subject for making engineered T cells (e.g., by apheresis). In some embodiments, a subject continues to receive a standard of care or approved treatment (e.g., at the dose used prior to receiving a provided treatment, at a reduced dose, or keeping only a limited number of individual therapeutic components from the original SOC treatment) while also receiving the presently provided treatment. In some embodiments, a subject does not continue to receive a standard of care or approved treatment while receiving the presently provided treatment. In some embodiments, a subject continues to receive a corticosteroid (e.g., a glucocorticoid, such as prednisone) at a reduced dose while also receiving the presently provided treatment. In some embodiments, a subject continues to receive a corticosteroid, such as prednisone at a treatment dose that is reduced to lower than or equal to 10 mg per day while also receiving the provided treatment. In some embodiments, a subject continues to receive a corticosteroid, such as prednisone at a treatment dose that is reduced to lower than or equal to 5 mg per day while also receiving the provided treatment or after having received the provided treatment. In some embodiments, a subject continues to receive a form of pyridostigmine (e.g., short and/or long acting forms) at a standard dose or reduced dose while also receiving the presently provided treatment. In some embodiments, a subject continues to receive a short acting form of pyridostigmine at a treatment dose of about 360 mg/day and a long acting form of pyridostigmine at a treatment dose of about 180 mg/day while also receiving the provided treatment. In some embodiments, an immunosuppressant (e.g., mycophenolate mofetil) is resumed after T cells are obtained from the subject for making engineered T cells (e.g., by apheresis) and withdrawn at least 2 days before lymphodepletion. In some embodiments, a subject continues to receive intravenous immunoglobulin therapy at a treatment dose that is reduced, e.g., at a dose of about 10 g to 20 g every 2-3 weeks, after having received the provided treatment.

In some embodiments, a subject receiving a presently provided treatment has previously received intravenous immunoglobulin (IVIG) therapy, and the IVIG therapy is washed out over a period of about 4 weeks or greater before T cells are obtained from the subject for making engineered T cells (e.g., via any method described herein, such as apheresis, or any common technique). In some embodiments, after T cells are obtained from the subject for making engineered T cells, IVIG therapy is resumed if required (e.g., as determined by a clinician to maintain disease stability). In some embodiments, a subject receiving a presently provided treatment has previously received plasma exchange therapy, and the plasma exchange therapy is washed out over a period of about 4 weeks or greater before T cells are obtained from the subject for making engineered T cells. In some embodiments, a subject receiving a presently provided treatment has previously received efgartigimod, and the efgartigimod is washed out over a period of about 4 weeks or greater before T cells are obtained from the subject for making engineered T cells. In some embodiments, a subject receiving a presently provided treatment has previously received rozanolixizumab, and the rozanolixizumab is washed out over a period of about 4 weeks or greater before T cells are obtained from the subject for making engineered T cells. In some embodiments, a subject receiving a presently provided treatment has previously received eculizumab, and the eculizumab is washed out over a period of about 4 weeks or greater before T cells are obtained from the subject for making engineered T cells. In some embodiments, a subject receiving a presently provided treatment has previously received ravulizumab, and the ravulizumab is washed out over a period of about 4 weeks or greater before T cells are obtained from the subject for making engineered T cells. In some embodiments, a subject receiving a presently provided treatment has previously received an anti-CD20 monoclonal antibody therapy (e.g., rituximab), and the anti-CD20 monoclonal antibody therapy is washed out over a period of about 3 months or greater before T cells are obtained from the subject for making engineered T cells. In some embodiments, a subject receiving a presently provided treatment has previously received cyclophosphamide, and the cyclophosphamide is washed out over a period of about 14 days or greater before T cells are obtained from the subject for making engineered T cells. In some embodiments, a subject receiving a presently provided treatment has previously received mycophenolate mofetil, and the mycophenolate mofetil is washed out over a period of about 7 days or greater before T cells are obtained from the subject for making engineered T cells. In some embodiments, after T cells are obtained from the subject for making engineered T cells, treatment with mycophenolate mofetil is resumed at the prior stable dose. In some embodiments, a subject receiving a presently provided treatment has previously received cyclosporine, and the cyclosporine is washed out over a period of about 7 days or greater before T cells are obtained from the subject for making engineered T cells. In some embodiments, after T cells are obtained from the subject for making engineered T cells, treatment with cyclosporine is resumed at the prior stable dose. In some embodiments, a subject receiving a presently provided treatment has previously received tacrolimus, and the tacrolimus is washed out over a period of about 7 days or greater before T cells are obtained from the subject for making engineered T cells. In some embodiments, after T cells are obtained from the subject for making engineered T cells, treatment with tacrolimus is resumed at the prior stable dose. In some embodiments, a subject receiving a presently provided treatment has previously received methotrexate, and the methotrexate is washed out over a period of about 14 days or greater before T cells are obtained from the subject for making engineered T cells. In some embodiments, after T cells are obtained from the subject for making engineered T cells, treatment with methotrexate is resumed at the prior stable dose. In some embodiments, a subject receiving a presently provided treatment has previously received azathioprine, and the azathioprine is washed out over a period of about 7 days or greater before T cells are obtained from the subject for making engineered T cells. In some embodiments, after T cells are obtained from the subject for making engineered T cells, treatment with azathioprine is resumed at the prior stable dose. In some embodiments, a subject receiving a presently provided treatment has previously received an investigational drug, and the investigational drug is washed out over a period of about 5 half-lives of the investigational drug or greater before T cells are obtained from the subject for making engineered T cells.

In some embodiments, a subject treated with a method or composition of the present disclosure (e.g., any subject described herein) has been diagnosed with concomitant myasthenia gravis and Lambert-Eaton myasthenic syndrome.

In some embodiments, a subject to receive a provided treatment will undergo a treatment scheme that comprises steps of: (1) reducing and/or washing out one or more previous myasthenia gravis medications (e.g., as described herein); (2) obtaining T cells from the subject for making engineered T cells (e.g., by apheresis); (3) optionally resuming one or more of the previous medications from step (1) if required (e.g., as determined by a clinician to maintain disease stability) for a duration of time; (4) optional lymphodepletion (e.g., via any method described herein); and (5) administration of the engineered T cells. In some embodiments, the time between step (1) and step (2) is about 7 days, about 8 days, about 9 days, about 10 days, about 11 days, about 12 days, about 13 days, about 14 days, about 3 weeks, about 4 weeks, about 8 weeks, about 12 weeks, or more. It will be understood by a skilled artisan that the time to effectively reduce and/or wash out one or more previous medications (e.g., the time between step (1) and step (2)) may vary, e.g., may be longer or shorter, or fall between values provided herein, depending on many factors including the type or class of previous medication and its half-life, among other things. In some embodiments, the time between step (2) and step (4) is about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, about 6 weeks, about 7 weeks, about 8 weeks, or more. It will be understood by a skilled artisan that the time between obtaining T cells from a subject for making engineered T cells (e.g., by apheresis) and lymphodepletion (e.g., the time between step (2) and step (4)) may vary, e.g., may be longer or shorter, or fall between values provided herein, depending on many factors including the health of the subject and the quality of the cells recovered for engineering, among other things. In some embodiments, the time between step (4) and step (5) is about 5 days, about 6 days, about 7 days, or more. It will be understood by a skilled artisan that the time between lymphodepletion and administration of the engineered T cells (e.g., the time between step (4) and step (5)) may vary, e.g., may be longer or shorter, or fall between values provided herein, depending on many factors including the method of lymphodepletion and the health of the subject, among other things. In some embodiments, a treatment scheme does not comprise a lymphodepletion step (e.g., a step (4)).

The present disclosure appreciates that therapeutic regimens for myasthenia gravis generally require chronic administration or administration of multiple treatment cycles to effectively treat the disease or disorder. The most common treatments include corticosteroids and immunosuppressive drugs, which can be very toxic to a subject. In some cases, these drugs can also suppress a subject's immune system, resulting in serious infections and/or adverse side effects in bone marrow, liver, and/or kidneys. Accordingly, standard of care treatments such as corticosteroid and immunosuppressive drug combinations present challenges to chronic use. In contrast, use of engineered T cells provided herein offers the premise of a single infusion possibly controlling disease for a prolonged period of time. Use of an engineered T cells as provided herein also permits repeated treatments (e.g., chronic administration, multiple treatment cycles, etc.) due to low toxicity profile and subsequent reduced side effects, e.g., as a result of the fully human nature the scFv, among other things. In many embodiments, use of the provided engineered T cells results in lower toxicity with subsequent reduced adverse effects over time and can be repeated in the future as needed.

In some embodiments, prior to receiving a provided treatment, a subject is administered an antihistamine and/or an antipyretic as a premedication. In some embodiments, an antihistamine is administered orally. In some embodiments, an antihistamine is administered intravenously. In some embodiments, an antihistamine is diphenhydramine. In some embodiments, diphenhydramine is administered at a dose in range of about 25 mg to about 50 mg. In some embodiments, diphenhydramine is administered orally, optionally 45-75 minutes (e.g., 1 hour) prior to administering a provided treatment. In some embodiments, diphenhydramine is administered intravenously, optionally 15-45 minutes (e.g., 30 minutes) prior to administering a provided treatment. In some embodiments, an antipyretic is acetaminophen. In some embodiments, acetaminophen is administered at a dose in a range of about 650 mg to about 1000 mg. In some embodiments, acetaminophen is administered orally or intravenously, optionally 15-45 minutes (e.g., 30 minutes) prior to administering a provided treatment. In some embodiments, a premedication (e.g., antihistamine and/or antipyretic) is administered about 15 minutes, about 30 minutes, about 45 minutes, about 1 hour, about 1.5 hours, about 2 hours, or more prior to administering a provided treatment.

In some embodiments, after receiving a presently provided treatment, or at the time of receiving a presently provided treatment, a subject is administered a prophylactic antimicrobial treatment and/or antiviral treatment. In some embodiments, the prophylactic antimicrobial treatment is a fluoroquinolone (e.g., levofloxacin), cefpodoxime, fluconazole, posaconazole, caspofungin, micafungin, trimethoprim-sulfamethoxazole, pentamidine, dapsone, or atovaquone. In some embodiments, the antiviral treatment is acyclovir or valacyclovir. In some embodiments, fluoroquinolone is administered at a dose of about 500 mg. In some embodiments, cefpodoxime is administered at a dose of about 200 mg. In some embodiments, fluconazole is administered at a dose of about 400 mg. In some embodiments, acyclovir is administered at a dose of about 400-800 mg. In some embodiments, valacyclovir is administered at a dose of about 500 mg. In some embodiments, pentamidine is administered at a dose of about 50 mg or about 100 mg. In some embodiments, a dapsone is administered at a dose of about 50 mg or about 100 mg. In some embodiments, atovaquone is administered at a dose of about 1500 mg. In some embodiments, the antimicrobial treatment and/or antiviral treatment are administered intravenously. In some embodiments, the antimicrobial treatment and/or antiviral treatment are administered orally. In some embodiments, the antimicrobial treatment and/or antiviral treatment are administered at least once per day, at least twice per day, at least three times per day, or more. In some embodiments, the antimicrobial treatment and/or antiviral treatment are administered at least once per day (e.g., at least twice per day, at least three times per day, etc.) for at least 3 days, at least 4 days, at least 5 days, at least 6, days, at least 7 days, at least 2 weeks, at least 4 weeks, at least 2 months, at least 4 months, at least 6 months, or longer. In some embodiments, the antimicrobial treatment and/or antiviral treatment are administered at least once per day (e.g., at least twice per day, at least three times per day, etc.) until the risk of microbial or viral infection has been minimized.

In some embodiments of any of the methods described herein, a step of administering comprises administering two or more doses of an engineered T cell (e.g., any engineered T cell described herein). In some embodiments, a step of administering comprises administering five or more doses of an engineered T cell. In some embodiments, a step of administering comprises administering ten or more doses of an engineered T cell. In some embodiments, a dose of an engineered T cell is fractionated such that the total dose is administered over the course at least two, three, four, five, six, seven, or more days.

The engineered T cells used in the methods of the present disclosure can be generated by a process disclosed herein. In some embodiments, an engineered T cell is generated by introducing into a T cell an engineered nucleic acid comprising a nucleic acid sequence encoding a CAR (e.g., any CAR described herein, e.g., an anti-CD19 CAR). In some embodiments, an engineered nucleic acid further comprises a promoter operably linked to a nucleic sequence encoding a CAR. In some embodiments, an engineered T cell is generated by further contacting the T cell with an effective amount of one or more agents that activate CD3 and CD28 under conditions that allow for stimulation of the T cell. In some embodiments, a T cell is obtained from a subject (e.g., an autologous or allogeneic subject), prior to a step of generating an engineered T cell and a step of administering the engineered T cell. An engineered T cell can be generated or made using any method of making an engineered T cell described herein, e.g., in the “Methods of Making Engineered T Cells” section above.

In some embodiments, the present disclosure provides for a method of reducing the number of B cells in a tissue in a subject having myasthenia gravis, the method comprising a step of administering an engineered T cell (e.g., any engineered T cell described herein). In some embodiments, the present disclosure provides for a method of reducing the number of B cells in a tissue in a subject having myasthenia gravis, the method comprising a step of administering an engineered T cell, wherein the engineered T cell is an anti-CD19 CAR T cell. In some embodiments, an anti-CD19 CAR T cell comprises a nucleic acid encoding an amino acid sequence as set forth in SEQ ID NO: 13. In some embodiments, an anti-CD19 CAR T cell comprises a nucleic acid as set forth in SEQ ID NO: 14.

In some embodiments, the present disclosure provides for a method of treating myasthenia gravis, the method comprising a step of administering an engineered T cell (e.g., any engineered T cell described herein). In some embodiments, the present disclosure provides for a method of treating myasthenia gravis, the method comprising a step of administering an engineered T cell, wherein the engineered T cell is an anti-CD19 CAR T cell. In some embodiments, an anti-CD19 CAR T cell comprises a nucleic acid encoding an amino acid sequence as set forth in SEQ ID NO: 13. In some embodiments, an anti-CD19 CAR T cell comprises a nucleic acid as set forth in SEQ ID NO: 14.

In some embodiments, the present disclosure provides for a method of treating Class I myasthenia gravis, the method comprising a step of administering an engineered T cell (e.g., any engineered T cell described herein). In some embodiments, the present disclosure provides for a method of treating Class I myasthenia gravis, the method comprising a step of administering an engineered T cell, wherein the engineered T cell is an anti-CD19 CAR T cell. In some embodiments, an anti-CD19 CAR T cell comprises a nucleic acid encoding an amino acid sequence as set forth in SEQ ID NO: 13. In some embodiments, an anti-CD19 CAR T cell comprises a nucleic acid as set forth in SEQ ID NO: 14.

In some embodiments, the present disclosure provides for a method of treating Class II myasthenia gravis, the method comprising a step of administering an engineered T cell (e.g., any engineered T cell described herein). In some embodiments, the present disclosure provides for a method of treating Class II myasthenia gravis, the method comprising a step of administering an engineered T cell, wherein the engineered T cell is an anti-CD19 CAR T cell. In some embodiments, an anti-CD19 CAR T cell comprises a nucleic acid encoding an amino acid sequence as set forth in SEQ ID NO: 13. In some embodiments, an anti-CD19 CAR T cell comprises a nucleic acid as set forth in SEQ ID NO: 14.

In some embodiments, the present disclosure provides for a method of treating Class III myasthenia gravis, the method comprising a step of administering an engineered T cell (e.g., any engineered T cell described herein). In some embodiments, the present disclosure provides for a method of treating Class III myasthenia gravis, the method comprising a step of administering an engineered T cell, wherein the engineered T cell is an anti-CD19 CAR T cell. In some embodiments, an anti-CD19 CAR T cell comprises a nucleic acid encoding an amino acid sequence as set forth in SEQ ID NO: 13. In some embodiments, an anti-CD19 CAR T cell comprises a nucleic acid as set forth in SEQ ID NO: 14.

In some embodiments, the present disclosure provides for a method of treating Class IV (e.g., Class IVa or Class IVb)myasthenia gravis, the method comprising a step of administering an engineered T cell (e.g., any engineered T cell described herein). In some embodiments, the present disclosure provides for a method of treating Class IV myasthenia gravis, the method comprising a step of administering an engineered T cell, wherein the engineered T cell is an anti-CD19 CAR T cell. In some embodiments, an anti-CD19 CAR T cell comprises a nucleic acid encoding an amino acid sequence as set forth in SEQ ID NO: 13. In some embodiments, an anti-CD19 CAR T cell comprises a nucleic acid as set forth in SEQ ID NO: 14.

In some embodiments, the present disclosure provides for a method of treating Class V myasthenia gravis, the method comprising a step of administering an engineered T cell (e.g., any engineered T cell described herein). In some embodiments, the present disclosure provides for a method of treating Class V myasthenia gravis, the method comprising a step of administering an engineered T cell, wherein the engineered T cell is an anti-CD19 CAR T cell. In some embodiments, an anti-CD19 CAR T cell comprises a nucleic acid encoding an amino acid sequence as set forth in SEQ ID NO: 13. In some embodiments, 15 an anti-CD19 CAR T cell comprises a nucleic acid as set forth in SEQ ID NO: 14.

In some embodiments, the present disclosure provides for a method of treating anti-AChR positive myasthenia gravis, the method comprising a step of administering an engineered T cell (e.g., any engineered T cell described herein). In some embodiments, the present disclosure provides for a method of treating anti-AChR positive myasthenia gravis, the method comprising a step of administering an engineered T cell, wherein the engineered T cell is an anti-CD19 CAR T cell. In some embodiments, an anti-CD19 CAR T cell comprises a nucleic acid encoding an amino acid sequence as set forth in SEQ ID NO: 13. In some embodiments, an anti-CD19 CAR T cell comprises a nucleic acid as set forth in SEQ ID NO: 14.

In some embodiments, the present disclosure provides for a method of treating anti-AChR negative myasthenia gravis, the method comprising a step of administering an engineered T cell (e.g., any engineered T cell described herein). In some embodiments, the present disclosure provides for a method of treating anti-AChR negative myasthenia gravis, the method comprising a step of administering an engineered T cell, wherein the engineered 30 T cell is an anti-CD19 CAR T cell. In some embodiments, an anti-CD19 CAR T cell comprises a nucleic acid encoding an amino acid sequence as set forth in SEQ ID NO: 13. In some embodiments, an anti-CD19 CAR T cell comprises a nucleic acid as set forth in SEQ ID NO: 14.

In some embodiments, the present disclosure provides for a method of treating refractory myasthenia gravis, the method comprising a step of administering an engineered T cell (e.g., any engineered T cell described herein). In some embodiments, the present disclosure provides for a method of treating refractory myasthenia gravis, the method comprising a step of administering an engineered T cell, wherein the engineered T cell is an anti-CD19 CAR T cell. In some embodiments, an anti-CD19 CAR T cell comprises a nucleic acid encoding an amino acid sequence as set forth in SEQ ID NO: 13. In some embodiments, an anti-CD19 CAR T cell comprises a nucleic acid as set forth in SEQ ID NO: 14.

In some embodiments, the present disclosure provides for a method of treating Class IV (e.g., Class IVa or Class IVb) refractory anti-AChR negative myasthenia gravis, the method comprising a step of administering an engineered T cell (e.g., any engineered T cell described herein). In some embodiments, the present disclosure provides for a method of treating Class IV refractory anti-AChR negative myasthenia gravis, the method comprising a step of administering an engineered T cell, wherein the engineered T cell is an anti-CD19 CAR T cell. In some embodiments, an anti-CD19 CAR T cell comprises a nucleic acid encoding an amino acid sequence as set forth in SEQ ID NO: 13. In some embodiments, an anti-CD19 CAR T cell comprises a nucleic acid as set forth in SEQ ID NO: 14.

In some embodiments, provided engineered T cells are administered using parenteral administration (e.g., intravenous administration). Methods and compositions of the present disclosure may be administered using any suitable method.

In some embodiments, administering of methods and compositions provided herein to a subject with myasthenia gravis results in improvement of the MG-Activity of Daily Living (MG-ADL), Quantitative MG (QMG), Myasthenia Gravis Composite (MGC), and/or Besinger scores, improvement in muscular strength as demonstrated by improved Forced Vital Capacity (FVC) and/or overall mobility (capacity to walk with limited to no assistance), and/or improvement of one or more patient-reported outcomes such as Myasthenia Gravis Quality of Life 15-item Scale—Revised (MGQOL15r), Myasthenia Gravis Foundation of America Post-Intervention Status (MGFA-PIS), Quality of Life in Neurological Disorders (Neuro-QOL) Fatigue Scale, and EuroQol Health 5-Dimensions (EQ-5D).

The MG-ADL focuses on relevant symptoms and performance of ADL in subjects with myasthenia gravis. The MG-ADL consists of 8 items, derived from symptom-based components of the original 13-item QMG, to assess disability secondary to ocular (2 items), bulbar (3 items), respiratory (1 item), and gross motor or limb (2 items) impairment related to the effects of myasthenia gravis. In the MG-ADL functional status instrument, each response is graded 0 (normal) to 3 (most severe), and the range of total MG-ADL score is 0 to 24. In some embodiments, the MG-ADL score of a subject is not increased 24 weeks after receiving the present method of treatment. In some embodiments, 24 weeks after receiving the present method of treatment, the MG-ADL score of a subject is decreased by at least 1, at least 2, at least 3, at least 4, at least 5, at least 6, at least 7, at least 8, at least 9, at least 10, at least 11, at least 12, at least 13, at least 14, at least 15, at least 16, at least 17, or at least 18. In some embodiments, 24 weeks after receiving the present method of treatment, the MG-ADL score of a subject is decreased by at least 40%, at least 50%, at least 60%, at least 70%, at least 80%, or at least 90% relative to the initial MG-ADL score of the subject.

The QMG scoring system is considered to be an objective evaluation of myasthenia gravis therapy and is based on a quantitative testing of muscle strength and endurance/fatigability of sentinel muscle groups. The QMG consists of 13 items: ocular (2 items), facial (1 item), bulbar (2 items), gross motor (6 items), axial (1 item) and respiratory (1 item); each graded 0 to 3, with 3 being the most severe. The range of total QMG score is 0-39. In some embodiments, the QMG score of a subject is not increased 12 weeks, 24 weeks, or 52 weeks after receiving the present method of treatment. In some embodiments, 12 weeks, 24 weeks, or 52 weeks after receiving the present method of treatment, the QMG score of a subject is decreased by at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, or at least 29. In some embodiments, 12 weeks, 24 weeks, or 52 weeks after receiving the present method of treatment, the QMG score of a subject is decreased by at least 40%, at least 50%, at least 60%, at least 70%, at least 80%, or at least 90% relative to the initial QMG score of the subject.

The MGC assesses 10 important functional areas most frequently affected by MG: ocular (2 items), axial (1 item), bulbar (3 items), respiratory (1 item), axial (1 item) and gross motor (2 items). The MGC scales are weighted for clinical significance that incorporates subject-reported outcomes, where higher scores indicate more functional impairment. The range of total MGC score is 0-50. In some embodiments, the MGC score of a subject is not increased 12 weeks, 24 weeks, or 52 weeks after receiving the present method of treatment. In some embodiments, 12 weeks, 24 weeks, or 52 weeks after receiving the present method of treatment, the MGC score of a subject is decreased by at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, or at least 37. In some embodiments, 12 weeks, 24 weeks, or 52 weeks after receiving the present method of treatment, the MGC score of a subject is decreased by at least 40%, at least 50%, at least 60%, at least 70%, at least 80%, or at least 90% relative to the initial QMG score of the subject.

The MG-QOL 15 is a health-related quality of life evaluative instrument specific to subjects with myasthenia gravis, completed by the subject. MG-QOL 15 is designed to provide information about subjects' perception of impairment and disability and the degree to which disease manifestations are tolerated and to be easy to administer and interpret (see Burns et al., Muscle Nerve. (2011) 43(1):14-8). Higher MG-QOL 15 scores indicate greater extent of and dissatisfaction with MG-related dysfunction. The Neuro-QOL Fatigue is a reliable and validated brief 19-item survey of fatigue, completed by the subject (Cella et al., Neurology. (2012) 78(23):1860-67). Higher Neuro-QOL Fatigue scores indicate greater fatigue and greater impact of MG on activities. The Besinger score (also referred to as Besinger-Toyka score) is a clinical score used to assess the severity of myasthenia gravis, and accordingly, may be used to assess clinical progression. The Besinger score is calculated using eight easy-to-collect criteria, including arm holding test, leg retention test, head holding test, vital capacity, a chewing/swallowing, mimic musculature, double vision, and ptosis. Exemplary descriptions of calculating the Besinger score can be found in Besinger, K. V. et al. (1983) Neurology 33(10): 1316-1321 and Jaretzki, R. J. et al. (2000) Neurology 55(1): 16-23.

In some embodiments, administering of methods and compositions provided herein to a subject with myasthenia gravis results in amelioration of one or more symptoms of myasthenia gravis in the subject. In some embodiments, administering of methods and compositions provided herein to a subject with myasthenia gravis results in a reduction in the number, severity, or frequency of one or more symptoms of myasthenia gravis in the subject (e.g., as compared to the number, severity, or frequency of the one or more symptoms of myasthenia gravis in the subject prior to receiving treatment with provided methods or compositions). In some embodiments, administering of methods and compositions provided herein to a subject with myasthenia gravis results in correction of the Trendelenburg sign. In some embodiments, a subject having myasthenia gravis having been administered an engineered T cell as described here can experience a reduction in inflammation and/or auto-antibody production.

A pharmaceutical composition useful in the method disclosed herein can contain an engineered T cell and a pharmaceutically acceptable carrier or buffer. In some embodiments, the pharmaceutical composition can be formulated in an injectable form (e.g., as solution and/or suspension). In some embodiments, a pharmaceutical composition comprising an engineered T cell as provided herein can further include phosphate buffered saline. Pharmaceutically acceptable carriers, fillers, and vehicles that can be used in a pharmaceutical composition described herein can include, without limitation, ion exchangers, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, and sodium chloride.

An effective dosage (e.g., for a provided T cell composition) to administer to a patient intravenously can vary depending on the severity of myasthenia gravis, the age and general health condition of a subject, excipient usage, the possibility of co-usage with other therapeutic treatments, and the judgment of the treating physician. An effective amount of an engineered T cell can be any amount that reduces inflammation and auto-antibody production within a subject having myasthenia gravis (e.g., via deletion or reduction of autoreactive B cells) without producing significant toxicity to the subject. In many embodiments, an effective dosage may also be dependent on the level of CAR expression in the provided engineered T cells and/or the percentage of engineered T cells within a provided composition. In some cases, engineered T cells can be a purified population of engineered T cells generated as described herein. In some cases, the purity of a population of engineered T cells can be assessed using any appropriate method, including, without limitation, flow cytometry. In some embodiments, purity of a population of engineered T cells can be assessed by quantifying the amount of T cells expressing the CAR relative to all the T cells in the population. In some cases, a population of engineered T cells to be administered to a subject can include a range of purities from about 5% to about 80%, about 10% to about 80%, about 15% to about 80%, about 20% to about 80%, about 30% to about 80%, about 40% to about 80%, about 50% to about 80%, about 60% to about 80%, about 5% to about 70%, about 5% to about 60%, about 5% to about 50%, about 5% to about 40%, about 5% to about 30%, about 5% to about 20%, about 5% to about 10%, about 70% to about 100%, from about 70% to about 90%, from about 70% to about 80%, from about 80% to about 90%, from about 90% to about 100%, from about 80% to about 100%, from about 80% to about 90%, or from about 90% to 100%. In some cases, a dosage of a provided therapy (e.g., number of engineered T cells to be administered) can adjusted based on the level of purity of the therapy.

In some embodiments, provided compositions (e.g., pharmaceutical compositions) of engineered T cells are administered at a dose of about 0.1×10⁸, about 0.2×10⁸, about 0.3×10⁸, about 0.4×10⁸, about 0.5×10⁸, about 0.6×10⁸, about 0.7×10⁸, about 0.8×10⁸, about 0.9×10⁸, about 1.0×10⁸, about 1.1×10⁸, about 1.2×10⁸, about 1.3×10⁸, about 1.4×10⁸, or about 1.5×10⁸ engineered cells. In some embodiments, provided compositions (e.g., pharmaceutical compositions) of engineered T cells are administered at a dose of about 0.5×10⁸ engineered cells. In some embodiments, provided compositions (e.g., pharmaceutical compositions) of engineered T cells are administered at a dose of about 1.0×10⁸ engineered cells.

In some embodiments, provided compositions (e.g., pharmaceutical compositions) of engineered T cells are administered at a dose of about 1×10⁵, about 1.5×10⁵, about 2×10⁵, about 2.5×10⁵, about 3×10⁵, about 3.5×10⁵, about 4×10⁵, about 4.5×10⁵, about 5×10⁵, about 5.5×10⁵, about 6×10⁵, about 6.5×10⁵, about 7×10⁵, about 7.5×10⁵, about 8×10⁵, about 8.5×10⁵, about 9×10⁵, about 9.5×10⁵, about 1×10⁶, about 1.2×10⁶, about 1.4×10⁶, about 1.6×10⁶, about 1.8×10⁶, about 2×10⁶, about 2.2×10⁶, or about 2.4×10⁶ engineered cells per kg of body weight. In some embodiments, provided compositions (e.g., pharmaceutical compositions) of engineered T cells are administered at a dose of about 7×10⁵ engineered cells per kg of body weight. In some embodiments, provided compositions (e.g., pharmaceutical compositions) of engineered T cells are administered at a dose of about 1.4×10⁶ engineered cells per kg of body weight.

The frequency of administration of an engineered T cell can be any frequency that reduces inflammation or auto-antibody production within a subject having myasthenia gravis (e.g., via deletion or reduction of autoreactive B cells) without producing toxicity to the subject. In some embodiments, the actual frequency of administration can vary depending on various factors including, without limitation, the effective amount, duration of treatment, use of multiple treatment agents, and severity of the condition may require an increase or decrease in frequency of administration.

An effective duration for administering a composition containing an anti-CD19 CAR T cell or a nucleic acid encoding the same can be any duration that reduces inflammation or auto-antibody production within the subject having myasthenia gravis (e.g., via deletion or reduction of autoreactive B cells) without producing toxicity to the subject. In some embodiments, the effective duration can vary from several days to several months. In some embodiments, the effective treatment duration for administering a composition containing an engineered T cell to treat myasthenia gravis can range in duration from about one month to about five years (e.g., from about two months to about five years, from about three months to about five years, from about six months to about five years, from about eight months to about five years, from about one year to about five years, from about one month to about four years, from about one month to about three years, from about one month to about two years, from about six months to about four years, from about six months to about three years, or from about six months to about two years). In some embodiments, the effective treatment duration is at least one year, two years, three years, or more. In some embodiments, a subject receives an infusion of a provided treatment and is cured (e.g., via initiation of an immune reset).

In some embodiments, a course of treatment and/or the severity of one or more symptoms related to myasthenia gravis can be monitored. Any appropriate method can be used to determine whether myasthenia gravis is being treated. For example, immunological techniques (e.g., ELISA) can be performed to determine if the level of auto-antibodies present within the subject being treated as described herein is reduced following the administration of an engineered T cell. Remission and relapse can be monitored by testing for one or more markers of myasthenia gravis, including but not limited to autoantibody titers and clinical signs of disease activity such as but not limited to Timed 25 Foot Walk, the Besinger score, the Quantitative MG score, the MGC score, and the MG Activities of Daily living score.

For continued efficacy, a subject can receive 1, 2, 3, 4, 5, 6, 7, 8, 9, 10, 11, 12, 13, 14, 15, 16, 17, 18, 19, 20, 30, 40, 50, or more doses of any engineered T cell described herein. In some embodiments, a subject receives at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 doses of an engineered T cell (e.g., a T cell expressing a CAR, e.g., a CAR comprising an amino acid sequence as set forth in SEQ ID NO: 13).

In some embodiments, administration of a provided method and composition results in a reduction (e.g., at least a 1% reduction, at least a 5% reduction, at least a 10% reduction, at least a 15% reduction, at least a 20% reduction, at least a 25% reduction, at least a 30% reduction, at least a 35% reduction, at least a 40% reduction, at least a 45% reduction, at least a 50% reduction, at least a 55% reduction, at least a 60% reduction, at least a 65% reduction, at least a 70% reduction, at least a 75% reduction, at least a 80% reduction, at least a 85% reduction, at least a 90% reduction, at least a 95% reduction, or at least a 99% reduction, or about a 1% reduction to about a 99% reduction, about a 1% reduction to about a 90% reduction, about a 1% reduction to about a 80% reduction, about a 1% reduction to about a 70% reduction, about a 1% reduction to about a 60% reduction, about a 1% reduction to about a 50% reduction, about a 1% reduction to about a 45% reduction, about a 1% reduction to about a 40% reduction, about a 1% reduction to about a 35% reduction, about a 1% reduction to about a 30% reduction, about a 1% reduction to about a 25% reduction, about a 1% reduction to about a 20% reduction, about a 1% reduction to about a 15% reduction, about a 1% reduction to about a 10% reduction, about a 1% reduction to about a 5% reduction, about a 5% reduction to about a 99% reduction, about a 5% reduction to about a 90% reduction, about a 5% reduction to about a 80% reduction, about a 5% reduction to about a 70% reduction, about a 5% reduction to about a 60% reduction, about a 5% reduction to about a 50% reduction, about a 5% reduction to about a 45% reduction, about a 5% reduction to about a 40% reduction, about a 5% reduction to about a 35% reduction, about a 5% reduction to about a 30% reduction, about a 5% reduction to about a 25% reduction, about a 5% reduction to about a 20% reduction, about a 5% reduction to about a 15% reduction, about a 5% reduction to about a 10% reduction, about a 10% reduction to about a 99% reduction, about a 10% reduction to about a 90% reduction, about a 10% reduction to about a 80% reduction, about a 10% reduction to about a 70% reduction, about a 10% reduction to about a 60% reduction, about a 10% reduction to about a 50% reduction, about a 10% reduction to about a 45% reduction, about a 10% reduction to about a 40% reduction, about a 10% reduction to about a 35% reduction, about a 10% reduction to about a 30% reduction, about a 10% reduction to about a 25% reduction, about a 10% reduction to about a 20% reduction, about a 10% reduction to about a 15% reduction, about a 15% reduction to about a 99% reduction, about a 15% reduction to about a 90% reduction, about a 15% reduction to about a 80% reduction, about a 15% reduction to about a 70% reduction, about a 15% reduction to about a 60% reduction, about a 15% reduction to about a 50% reduction, about a 15% reduction to about a 45% reduction, about a 15% reduction to about a 40% reduction, about a 15% reduction to about a 35% reduction, about a 15% reduction to about a 30% reduction, about a 15% reduction to about a 25% reduction, about a 15% reduction to about a 20% reduction, about a 20% reduction to about a 99% reduction, about a 20% reduction to about a 90% reduction, about a 20% reduction to about a 80% reduction, about a 20% reduction to about a 70% reduction, about a 20% reduction to about a 60% reduction, about a 20% reduction to about a 50% reduction, about a 20% reduction to about a 45% reduction, about a 20% reduction to about a 40% reduction, about a 20% reduction to about a 35% reduction, about a 20% reduction to about a 30% reduction, about a 20% reduction to about a 25% reduction, about a 25% reduction to about a 99% reduction, about a 25% reduction to about a 90% reduction, about a 25% reduction to about a 80% reduction, about a 25% reduction to about a 70% reduction, about a 25% reduction to about a 60% reduction, about a 25% reduction to about a 50% reduction, about a 25% reduction to about a 45% reduction, about a 25% reduction to about a 40% reduction, about a 25% reduction to about a 35% reduction, about a 25% reduction to about a 30% reduction, about a 30% reduction to about a 99% reduction, about a 30% reduction to about a 90% reduction, about a 30% reduction to about a 80% reduction, about a 30% reduction to about a 70% reduction, about a 30% reduction to about a 60% reduction, about a 30% reduction to about a 50% reduction, about a 30% reduction to about a 45% reduction, about a 30% reduction to about a 40% reduction, about a 30% reduction to about a 35% reduction, about a 35% reduction to about a 99% reduction, about a 35% reduction to about a 90% reduction, about a 35% reduction to about a 80% reduction, about a 35% reduction to about a 70% reduction, about a 35% reduction to about a 60% reduction, about a 35% reduction to about a 50% reduction, about a 35% reduction to about a 45% reduction, about a 35% reduction to about a 40% reduction, about a 40% reduction to about a 99% reduction, about a 40% reduction to about a 90% reduction, about a 40% reduction to about a 80% reduction, about a 40% reduction to about a 70% reduction, about a 40% reduction to about a 60% reduction, about a 40% reduction to about a 50% reduction, about a 40% reduction to about a 45% reduction, about a 45% reduction to about a 99% reduction, about a 45% reduction to about a 90% reduction, about a 45% reduction to about a 80% reduction, about a 45% reduction to about a 70% reduction, about a 45% reduction to about a 60% reduction, about a 45% reduction to about a 50% reduction, about a 50% reduction to about a 99% reduction, about a 50% reduction to about a 90% reduction, about a 50% reduction to about a 80% reduction, about a 50% reduction to about a 70% reduction, about a 50% reduction to about a 60% reduction, about a 60% reduction to about a 99% reduction, about a 60% reduction to about a 90% reduction, about a 60% reduction to about a 80% reduction, about a 60% reduction to about a 70% reduction, about a 70% reduction to about a 99% reduction, about a 70% reduction to about a 90% reduction, about a 70% reduction to about a 80% reduction, about a 80% reduction to about a 99% reduction, about a 80% reduction to about a 90% reduction, or about a 90% reduction to about a 99% reduction) in the number of B cells in a tissue of the subject (e.g., in peripheral blood) having myasthenia gravis, e.g., as compared to the levels in the subject prior to treatment or the levels in a similar subject not treated or receiving a different treatment. In some embodiments, after deep measured depletion of B cells in a tissue of a subject (e.g., in peripheral blood), B cell counts may increase and substantially recover to normal levels, e.g., as compared to levels in the subject prior to treatment or the levels in a similar subject not treated or receiving a different treatment or any other suitable control. In some embodiments, B cell counts will substantially recover to normal levels after about 3, 4, 5, 6, 7, 8, 9, 10, 11, or 12 months. In some embodiment, recovered B cells will show a sustained naive phenotype profile, indicating the potential for an immune reset and a long-term functional cure of the subject. In some embodiments, the amount of anti-AChR antibodies produced by the recovered B cells and measured in serum is reduced by at least 20%, 30%, 40%, 50%, 60%, 70%, 80%, 90%, 95%, or 99% relative to the amount of anti-AChR antibodies prior to the provided treatment. In some embodiments, the amount of anti-VGCC N type antibodies produced by the recovered B cells and measured in serum is reduced by at least 20%, 30%, 40%, 50%, 60%, 70%, 80%, 90%, 95%, or 99% relative to the amount of anti-VGCC N type antibodies prior to the provided treatment. In some embodiments, the amount of anti-MuSK antibodies produced by the recovered B cells and measured in serum is reduced by at least 20%, 30%, 40%, 50%, 60%, 70%, 80%, 90%, 95%, or 99% relative to the amount of anti-MuSK antibodies prior to the provided treatment. In some embodiments, the amount of anti-LRP4 antibodies produced by the recovered B cells and measured in serum is reduced by at least 20%, 30%, 40%, 50%, 60%, 70%, 80%, 90%, 95%, or 99% relative to the amount of anti-LRP4 antibodies prior to the provided treatment. Without wishing to be bound by theory, it is contemplated that the B cell depletion caused by the CAR T cells may lead to an immune reset, e.g., as evidenced by the durable absence of symptoms of the disease even in the presence of reconstituted B cell numbers.

In some embodiments, administration of methods and compositions described herein result in a reduction (e.g., at least a 1% reduction, at least a 5% reduction, at least a 10% reduction, at least a 15% reduction, at least a 20% reduction, at least a 25% reduction, at least a 30% reduction, at least a 35% reduction, at least a 40% reduction, at least a 45% reduction, at least a 50% reduction, at least a 55% reduction, at least a 60% reduction, at least a 65% reduction, at least a 70% reduction, at least a 75% reduction, at least a 80% reduction, at least a 85% reduction, at least a 90% reduction, at least a 95% reduction, or at least a 99% reduction, or about a 1% reduction to about a 99% reduction (or any of the subranges of this range described herein) in the level of auto-antibodies in the subject having myasthenia gravis, e.g., as compared to the levels in the subject prior to treatment or the levels in a similar subject not treated or receiving a different treatment.

### Lambert-Eaton Myasthenic Syndrome (LEMS)

Provided herein are methods and compositions for reducing the number of B cells in a subject having Lambert-Eaton myasthenic syndrome (LEMS). In some embodiments, the present disclosure provides a method of reducing the number of B cells producing autoantibodies that mediate autoimmunity in a tissue in a subject having LEMS, the method comprising administering a therapeutically effective amount of any engineered T cell described herein to the subject. Also provided herein are methods of treating a subject having LEMS, the method comprising administering a therapeutically effective amount of an engineered T cell (e.g., any engineered T cell described herein, e.g., an anti-CD19 CAR T cell) to the subject.

Provided methods and compositions for reducing the number of B cells may be used in a subject with myasthenia gravis and LEMS (e.g., the subject has been diagnosed with concomitant myasthenia gravis and LEMS).

In some embodiments, a subject receiving a provided treatment has previously received a standard of care or approved treatment for LEMS that was ineffective and/or caused one or more adverse side effects. For example, in some embodiments, a subject receiving a presently provided treatment has previously been treated with IVIG, prednisone, plasma exchange, azathioprine, mycophenolate mofetil, rituximab, or cyclosporine.

### Stiff Person Syndrome (SPS)

Provided herein are methods and compositions for reducing the number of B cells in a subject having stiff person syndrome (SPS). Stiff-person syndrome (SPS) is characterized by progressive rigidity and muscle spasms, which usually affect axial and limb muscles. In some embodiments, provided herein are methods and compositions for reducing the number of B cells in a subject having refractory SPS. In some embodiments, the present disclosure provides a method of reducing the number of B cells producing autoantibodies that mediate autoimmunity in a tissue in a subject having SPS (e.g., refractory SPS), the method comprising administering a therapeutically effective amount of any engineered T cell described herein to the subject. In some embodiments, the present disclosure provides methods and compositions for reducing pathogenic autoantibodies associated with SPS, such as anti-amphiphysin autoantibodies, anti-GAD autoantibodies (e.g., anti-GAD65 autoantibodies), anti-GABARAP autoantibodies, anti-GlyR autoantibodies, etc. Also provided herein are methods of treating a subject having SPS, the method comprising administering a therapeutically effective amount of an engineered T cell (e.g., any engineered T cell described herein, e.g., an anti-CD19 CAR T cell) to the subject. In some embodiments, the SPS is classic SPS. In some embodiments, the SPS is partial SPS (e.g., any partial SPS variant). In some embodiments, the SPS is progressive encephalomyelitis with rigidity and myoclonus (PERM). In some embodiments, the SPS is SPS Plus. In some embodiments, the SPS is anti-GAD65 antibody positive.

In some embodiments, a course of treatment and/or the severity of one or more symptoms related to SPS can be monitored. Any appropriate method can be used to determine whether SPS is being treated. For example, immunological techniques (e.g., ELISA) can be performed to determine if the level of auto-antibodies present within the subject being treated as described herein is reduced following the administration of an engineered T cell. Remission and relapse can be monitored by testing for one or more markers of SPS, including but not limited to autoantibody titers and clinical signs of disease activity such as but not limited to the Modified Ashworth Scale (MAS), numeric rating scale (NRS), fatigue severity scale, stiffness index, and electrophysical assessment.

In some embodiments, a subject receiving a provided treatment (e.g., any engineered nucleic acid, engineered T cell, or CAR provided herein) has previously been treated with a lymphodepletion agent (e.g., cyclophosphamide and/or fludarabine). In some embodiments, a subject receiving a provided treatment has previously received a standard of care or approved treatment for SPS that was ineffective and/or caused one or more adverse side effects. For example, in some embodiments, a subject receiving a presently provided treatment has previously been treated with a benzodiazepine (e.g., diazepam, clonazepam, alprazolam, lorazepam, temazepam, etc.). In some embodiments, a subject receiving a presently provided treatment has previously been treated with an anti-spasticity agent (e.g., baclofen, etc.). In some embodiments, a subject receiving a presently provided treatment has previously been treated with an anti-epileptic (e.g., gabapentin, vigabatrin, tiagabine, tizanidine, dantrolene, etc.). In some embodiments, a subject receiving a presently provided treatment has previously been treated with botulinum toxin. In some embodiments, a subject receiving a presently provided treatment has previously been treated with IVIg. In some embodiments, a subject receiving a presently provided treatment has previously been treated with an anti-CD20 antibody (e.g., rituximab). In some embodiments, a subject receiving a presently provided treatment has previously been treated with autologous hematopoietic stem cell transplantation (auto-HSCT). In some embodiments, a subject receiving a presently provided treatment has previously been treated with plasmapheresis. In some embodiments, a subject receiving a presently provided treatment has previously been treated with corticosteroids. In some embodiments, a subject receiving a presently provided treatment has previously been treated with an immunosuppressant.

In some embodiments, a subject receiving a presently provided treatment has previously received a standard of care or approved treatment for SPS (e.g., any common treatment described herein, e.g., those discussed above), and the standard of care or approved treatment dose is reduced (e.g., by tapering) or the standard of care or approved treatment is terminated (e.g., by washing out) before T cells are obtained from the subject for making engineered T cells (e.g., via any method described herein). In some embodiments, a standard of care or approved treatment dose is reduced or the standard of care or approved treatment is terminated at least 1 week, at least 2 weeks, at least 3 weeks, at least 4 weeks, at least 5 weeks, at least 6 weeks, at least 7 weeks, or at least 8 weeks before T cells are obtained from the subject for making engineered T cells (e.g., by apheresis). In some embodiments, a standard of care or approved treatment dose is reduced at least 6 weeks prior to a subject receiving a provided treatment. In some embodiments, a subject continues to receive a standard of care or approved treatment (e.g., at a standard dose, at a reduced dose, and/or keeping only a limited number of individual therapeutic components from the original SOC treatment) while also receiving the presently provided treatment. In some embodiments, a subject does not continue to receive a standard of care or approved treatment while receiving the presently provided treatment.

In some embodiments, a subject to receive a provided treatment will undergo a treatment scheme that comprises steps of: (1) reducing and/or washing out one or more previous SPS medications (e.g., as described herein); (2) obtaining T cells from the subject for making engineered T cells (e.g., by apheresis); (3) optionally resuming one or more of the previous medications from step (1) if required (e.g., as determined by a clinician to maintain disease stability) for a duration of time; (4) optional lymphodepletion (e.g., via any method described herein); and (5) administration of the engineered T cells. In some embodiments, the time between step (1) and step (2) is about 7 days, about 8 days, about 9 days, about 10 days, about 11 days, about 12 days, about 13 days, about 14 days, about 3 weeks, about 4 weeks, about 8 weeks, about 12 weeks, or more. It will be understood by a skilled artisan that the time to effectively reduce and/or wash out one or more previous medications (e.g., the time between step (1) and step (2)) may vary, e.g., may be longer or shorter, or fall between values provided herein, depending on many factors including the type or class of previous medication and its half-life, among other things. In some embodiments, the time between step (2) and step (4) is about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, about 6 weeks, about 7 weeks, about 8 weeks, or more. It will be understood by a skilled artisan that the time between obtaining T cells from a subject for making engineered T cells (e.g., by apheresis) and lymphodepletion (e.g., the time between step (2) and step (4)) may vary, e.g., may be longer or shorter, or fall between values provided herein, depending on many factors including the health of the subject and the quality of the cells recovered for engineering, among other things. In some embodiments, the time between step (4) and step (5) is about 5 days, about 6 days, about 7 days, or more. It will be understood by a skilled artisan that the time between lymphodepletion and administration of the engineered T cells (e.g., the time between step (4) and step (5)) may vary, e.g., may be longer or shorter, or fall between values provided herein, depending on many factors including the method of lymphodepletion and the health of the subject, among other things. In some embodiments, a treatment scheme does not comprise a lymphodepletion step (e.g., a step (4)).

In some embodiments, the present disclosure provides for a method of reducing the number of B cells in a tissue in a subject having SPS, the method comprising a step of administering an engineered T cell (e.g., any engineered T cell described herein). In some embodiments, the present disclosure provides for a method of reducing the number of B cells in a tissue in a subject having SPS, the method comprising a step of administering an engineered T cell, wherein the engineered T cell is an anti-CD19 CAR T cell. In some embodiments, an anti-CD19 CAR T cell comprises a nucleic acid encoding an amino acid sequence as set forth in SEQ ID NO: 13. In some embodiments, an anti-CD19 CAR T cell comprises a nucleic acid as set forth in SEQ ID NO: 14.

In some embodiments, the present disclosure provides for a method of treating SPS (e.g., refractory SPS), the method comprising a step of administering an engineered T cell (e.g., any engineered T cell described herein). In some embodiments, the present disclosure provides for a method of treating SPS (e.g., refractory SPS), the method comprising a step of administering an engineered T cell, wherein the engineered T cell is an anti-CD19 CAR T cell. In some embodiments, an anti-CD19 CAR T cell comprises a nucleic acid encoding an amino acid sequence as set forth in SEQ ID NO: 13. In some embodiments, an anti-CD19 CAR T cell comprises a nucleic acid as set forth in SEQ ID NO: 14.

In some embodiments, the present disclosure provides for a method of treating anti-glutamic acid decarboxylase (GAD) (e.g., anti-GAD65) positive SPS, the method comprising a step of administering an engineered T cell (e.g., any engineered T cell described herein). In some embodiments, the present disclosure provides for a method of treating anti-GAD (e.g., anti-GAD65) positive SPS, the method comprising a step of administering an engineered T cell, wherein the engineered T cell is an anti-CD19 CAR T cell. In some embodiments, an anti-CD19 CAR T cell comprises a nucleic acid encoding an amino acid sequence as set forth in SEQ ID NO: 13. In some embodiments, an anti-CD19 CAR T cell comprises a nucleic acid as set forth in SEQ ID NO: 14.

In some embodiments, the titer of anti-GAD65 antibodies in a subject prior to receiving a treatment of the present disclosure is about 15 nmole/L, about 16 nmole/L, about 17 nmole/L, about 18 nmole/L, about 19 nmole/L, about 20 nmole/L, about 21 nmole/L, about 22 nmole/L, about 25 nmole/L, or greater. In some embodiments, the titer of anti-GAD65 antibodies is about 20 nmole/L

In some embodiments, provided compositions (e.g., pharmaceutical compositions) of engineered T cells are administered at a dose of about 0.1×10⁸, about 0.2×10⁸, about 0.3×10⁸, about 0.4×10⁸, about 0.5×10⁸, about 0.6×10⁸, about 0.7×10⁸, about 0.8×10⁸, about 0.9×10⁸, about 1.0×10⁸, about 1.1×10⁸, about 1.2×10⁸, about 1.3×10⁸, about 1.4×10⁸, or about 1.5×10⁸ engineered cells. In some embodiments, provided compositions (e.g., pharmaceutical compositions) of engineered T cells are administered at a dose of about 0.5×10⁸ engineered cells. In some embodiments, provided compositions (e.g., pharmaceutical compositions) of engineered T cells are administered at a dose of about 1.0×10⁸ engineered cells.

In some embodiments, provided compositions (e.g., pharmaceutical compositions) of engineered T cells are administered at a dose of about 1×10⁵, about 1.5×10⁵, about 2×10⁵, about 2.5×10⁵, about 3×10⁵, about 3.5×10⁵, about 4×10⁵, about 4.5×10⁵, about 5×10⁵, about 5.5×10⁵, about 6×10⁵, about 6.5×10⁵, about 7×10⁵, about 7.5×10⁵, about 8×10⁵, about 8.5×10⁵, about 9×10⁵, about 9.5×10⁵, about 1×10⁶, about 1.2×10⁶, about 1.4×10⁶, about 1.6×10⁶, about 1.8×10⁶, about 2×10⁶, about 2.2×10⁶, or about 2.4×10⁶ engineered cells per kg of body weight. In some embodiments, provided compositions (e.g., pharmaceutical compositions) of engineered T cells are administered at a dose of about 7×10⁵ engineered cells per kg of body weight. In some embodiments, provided compositions (e.g., pharmaceutical compositions) of engineered T cells are administered at a dose of about 1.4×10⁶ engineered cells per kg of body weight.

The frequency of administration of an engineered T cell can be any frequency that reduces inflammation or auto-antibody production within a subject having SPS (e.g., via deletion or reduction of autoreactive B cells) without producing toxicity to the subject. In some embodiments, the actual frequency of administration can vary depending on various factors including, without limitation, the effective amount, duration of treatment, use of multiple treatment agents, and severity of the condition may require an increase or decrease in frequency of administration.

An effective duration for administering a composition containing an anti-CD19 CAR T cell or a nucleic acid encoding the same can be any duration that reduces inflammation or auto-antibody production within the subject having SPS (e.g., via deletion or reduction of autoreactive B cells) without producing toxicity to the subject. In some embodiments, the effective duration can vary from several days to several months. In some embodiments, the effective treatment duration for administering a composition containing an engineered T cell to treat SPS can range in duration from about one month to about five years (e.g., from about two months to about five years, from about three months to about five years, from about six months to about five years, from about eight months to about five years, from about one year to about five years, from about one month to about four years, from about one month to about three years, from about one month to about two years, from about six months to about four years, from about six months to about three years, or from about six months to about two years). In some embodiments, the effective treatment duration is at least one year, two years, three years, or more. In some embodiments, a subject receives an infusion of a provided treatment and is cured (e.g., via initiation of an immune reset).

In some embodiments, administering of methods and compositions provided herein to a subject with SPS results in improvement as determined by modified Ashworth scale (MAS), numeric rating scale (NRS), fatigue severity scale (FSS), stiffness index, T25-FW, Modified Rankin Scale, Hauser Ambulation Index, Heightened Sensitivity Scale, 6-minute walk test, 36 item short form survey (SF-36), and/or electrophysical assessment.

The modified Ashworth scale (MAS) purpose is to grade muscle spasticity. The MAS is a 6-point scale, with scores ranging from 0 to 4, where lower scores represent normal muscle tone and higher scores represent spasticity or increased resistance to passive movement. The scale is as follows: 0: No increase in muscle tone; 1: slight increase in muscle tone, with a catch and release or minimal resistance at the end of the range of motion when an affected part(s) is moved in flexion or extension; 1+: slight increase in muscle tone, manifested as a catch, followed by minimal resistance through the remainder (less than half) of the range of motion; 2: a marked increase in muscle tone throughout most of the range of motion, but affected part(s) are still easily moved; 3: considerable increase in muscle tone, passive movement difficult; and 4: affected part(s) rigid in flexion or extension. In some embodiments, the MAS score of a subject is not increased after receiving the present method of treatment. In some embodiments, about 14 days or more after receiving the present method of treatment, the MAS score of a subject is decreased by at least 1, at least 2, at least 3, at least 4, or at least 5 points.

The numeric rating scale (NRS) is a scale used to rate pain. The scale ranges from 0 to 10, with 0 being no pain and 10 being the worst pain imaginable. In some embodiments, the NRS score of a subject is not increased after receiving the present method of treatment. In some embodiments, about 1 month, about 2 month, or more after receiving the present method of treatment, the NRS score of a subject is decreased by at least 1, at least 2, at least 3, at least 4, at least 5 points, at least 6, at least 7, at least 8, at least 9, or at least 10 points.

The fatigue severity score (FSS) is a nine-item instrument designed to assess fatigue as a symptom of a variety of different chronic conditions and disorders. The scale addresses fatigue's effects on daily functioning, querying its relationship to motivation, physical activity, work, family, and social life, and asking respondents to rate the ease with which they are fatigued and the degree to which the symptom poses a problem for them. Respondents use a scale ranging from 1 (“completely disagree”) to 7 (“completely agree”) to indicate their agreement with nine statements about fatigue. The minimum score is 9 and maximum score possible is 63. Higher scores on the scale are indicative of more severe fatigue. In some embodiments, the FSS of a subject is not increased after receiving the present method of treatment. In some embodiments, about 1 month, about 2 months, about 3 months, about 4 months, or more after receiving the present method of treatment, the FSS of a subject is decreased by at least 1, at least 2, at least 3, at least 4, at least 5, at least 10, at least 15, at least 20, at least 25, at least 30, at least 35, at least 40, at least 45, at least 50, or more points.

The T25-FW is a quantitative mobility and leg function performance test based on a timed 25-foot walk. In an exemplary T25-FW test, a patient may be directed to one end of a clearly marked 25-foot course and is instructed to walk 25 feet as quickly as possible, but safely. The time is calculated from the initiation of the instruction and ends when the patient has reached the 25-foot mark. The task is immediately administered again by having the patient walk back the same distance. In some cases, a patient may use assistive devices when doing this task. In some embodiments, the T25-FW time is not increased after receiving the present method of treatment. In some embodiments, the T25-FW time is decreased by at least 5%, at least 12%, at least 15%, at least 18%, at least 20%, at least 22%, at least 25%, at least 28%, at least 30%, or more after receiving the present method of treatment, as compared to the T25-FW time measured prior to receiving the treatment.

The modified Rankin Scale (mRS) is a commonly used scale for measuring the degree of disability or dependence in the daily activities of people who have suffered a stroke or other causes of neurological disability. The scale runs from 0-6, running from perfect health without symptoms to death: 0—No symptoms; 1—No significant disability, able to carry out all usual activities, despite some symptoms; 2—Slight disability, able to look after own affairs without assistance, but unable to carry out all previous activities; 3—Moderate disability, requires some help, but able to walk unassisted; 4—Moderately severe disability, unable to attend to own bodily needs without assistance, and unable to walk unassisted; 5—Severe disability, requires constant nursing care and attention, bedridden, incontinent; and 6—Dead. In some embodiments, the mRS score is not increased after receiving the present method of treatment. In some embodiments, the mRS score is decreased by 1, 2, 3, 4, or 5 points after receiving the present method of treatment, as compared to the mRS score measured prior to receiving the treatment.

The Hauser Ambulation Index is used to assess mobility by evaluating the time and degree of assistance required to walk 25 feet. Scores range from 0 (asymptomatic and fully active) to 10 (bedridden). The patient is asked to walk a marked 25-foot course as quickly and safely as possible. The examiner records the time and type of assistance (e.g., cane, walker, crutches) needed. The Hauser Ambulation Index scores include: 0=Asymptomatic; fully active; 1=Walks normally, but reports fatigue that interferes with athletic or other demanding activities; 2=Abnormal gait or episodic imbalance; gait disorder is noticed by family and friends; able to walk 25 feet (8 meters) in 10 seconds or less; 3=Walks independently; able to walk 25 feet in 20 seconds or less; 4=Requires unilateral support (cane or single crutch) to walk; walks 25 feet in 20 seconds or less; 5=Requires bilateral support (canes, crutches, or walker) and walks 25 feet in 20 seconds or less; or requires unilateral support but needs more than 20 seconds to walk 25 feet; 6=Requires bilateral support and more than 20 seconds to walk 25 feet; may use wheelchair* on occasion; 7=Walking limited to several steps with bilateral support; unable to walk 25 feet; may use wheelchair* for most activities; 8=Restricted to wheelchair; able to transfer self independently; and 9=Restricted to wheelchair; unable to transfer self independently. In some embodiments, the Hauser Ambulation Index is not increased after receiving the present method of treatment. In some embodiments, the Hauser Ambulation Index is decreased by 1, 2, 3, 4, 5, 6, 7, 8, or 9 points after receiving the present method of treatment, as compared to the Hauser Ambulation Index measured prior to receiving the treatment.

The 6-minute walk test is a sub-maximal exercise test used to assess aerobic capacity and endurance. In this test, the distance covered over a time of 6 minutes is used as the outcome by which to compare changes in performance capacity. As an example, a 6-minute walk test may be carried out by asking a patient or subject to walk as far as possible for 6 minutes. The patient or subject is permitted to slow down, stop, and/or rest as necessary during the test. In some embodiments, the 6-minute walk test measurement is not increased after receiving the present method of treatment. In some embodiments, the 6-minute walk test measurement is decreased by at least 5%, at least 12%, at least 15%, at least 18%, at least 20%, at least 22%, at least 25%, at least 28%, at least 30%, or more after receiving the present method of treatment, as compared to the 6-minute walk test measurement taken prior to receiving the treatment.

The stiffness index score determines and quantifies stiffness as follows: 0: Absent stiffness; 1: Stiffness in face; 2: Stiffness in arms; 3: Stiffness in upper trunk; 4: Stiffness in abdomen; 5: Stiffness in lower trunk; 6: Stiffness in legs. The presence of each item adds one point (maximum score 6). In some embodiments, the stiffness index score is not increased after receiving the present method of treatment. In some embodiments, the stiffness index score is decreased by 1, 2, 3, 4, 5, or 6 points after receiving the present method of treatment, as compared to the stiffness index score measured prior to receiving the treatment.

The heightened sensitivity scale determines and quantifies the events that trigger stiffness and spasms as follows: 1: Noise-induced stiffness and cramps; 2: Visually induced stiffness and cramps; 3: Somato-sensory-induced stiffness and cramps; 4: Voluntary activity induced spasms; 5: Emotional upset and “stress”-induced spasms; 6: Awakening due to nocturnal spasms; 7: Untriggered cramps and spasms. The presence of each item adds one point (maximum score 7). In some embodiments, the heightened sensitivity scale score is not increased after receiving the present method of treatment. In some embodiments, the heightened sensitivity scale score is decreased by 1, 2, 3, 4, 5, 6, or 7 points after receiving the present method of treatment, as compared to the heightened sensitivity scale score measured prior to receiving the treatment.

Use of the Stiffness Index, the T25-FW test, and the Heightened Sensitivity Scale to diagnose and assess severity of diseases and to deduce clinical efficiency is understood by those skilled in the art. Some examples of using the Stiffness Index and Heightened Sensitivity Scale in a clinical setting can be found in Celli S. I., et al. Neurol Neuroimmunol Neuroinflamm. 2024 March; 11(2):e200197. doi: 10.1212/NXI.0000000000200197; Dalakas M. C., et al. Ann Neurol. 2017 August; 82(2):271-277. doi: 10.1002/ana.25002; and Dalakas M. C., et al. N Engl J Med. 2001 Dec. 27; 345(26):1870-6. doi: 10.1056/NEJMoa01167.

In some embodiments, administration of a provided method and composition results in a reduction in the number of B cells in a tissue of the subject (e.g., in peripheral blood) having SPS, e.g., as compared to the levels in the subject prior to treatment or the levels in a similar subject not treated or receiving a different treatment. The level of reduction can be at least a 1% reduction, at least a 5% reduction, at least a 10% reduction, at least a 15% reduction, at least a 20% reduction, at least a 25% reduction, at least a 30% reduction, at least a 35% reduction, at least a 40% reduction, at least a 45% reduction, at least a 50% reduction, at least a 55% reduction, at least a 60% reduction, at least a 65% reduction, at least a 70% reduction, at least a 75% reduction, at least a 80% reduction, at least a 85% reduction, at least a 90% reduction, at least a 95% reduction, or at least a 99% reduction, or about a 1% reduction to about a 99% reduction, about a 1% reduction to about a 90% reduction, about a 1% reduction to about a 80% reduction, about a 1% reduction to about a 70% reduction, about a 1% reduction to about a 60% reduction, about a 1% reduction to about a 50% reduction, about a 1% reduction to about a 45% reduction, about a 1% reduction to about a 40% reduction, about a 1% reduction to about a 35% reduction, about a 1% reduction to about a 30% reduction, about a 1% reduction to about a 25% reduction, about a 1% reduction to about a 20% reduction, about a 1% reduction to about a 15% reduction, about a 1% reduction to about a 10% reduction, about a 1% reduction to about a 5% reduction, about a 5% reduction to about a 99% reduction, about a 5% reduction to about a 90% reduction, about a 5% reduction to about a 80% reduction, about a 5% reduction to about a 70% reduction, about a 5% reduction to about a 60% reduction, about a 5% reduction to about a 50% reduction, about a 5% reduction to about a 45% reduction, about a 5% reduction to about a 40% reduction, about a 5% reduction to about a 35% reduction, about a 5% reduction to about a 30% reduction, about a 5% reduction to about a 25% reduction, about a 5% reduction to about a 20% reduction, about a 5% reduction to about a 15% reduction, about a 5% reduction to about a 10% reduction, about a 10% reduction to about a 99% reduction, about a 10% reduction to about a 90% reduction, about a 10% reduction to about a 80% reduction, about a 10% reduction to about a 70% reduction, about a 10% reduction to about a 60% reduction, about a 10% reduction to about a 50% reduction, about a 10% reduction to about a 45% reduction, about a 10% reduction to about a 40% reduction, about a 10% reduction to about a 35% reduction, about a 10% reduction to about a 30% reduction, about a 10% reduction to about a 25% reduction, about a 10% reduction to about a 20% reduction, about a 10% reduction to about a 15% reduction, about a 15% reduction to about a 99% reduction, about a 15% reduction to about a 90% reduction, about a 15% reduction to about a 80% reduction, about a 15% reduction to about a 70% reduction, about a 15% reduction to about a 60% reduction, about a 15% reduction to about a 50% reduction, about a 15% reduction to about a 45% reduction, about a 15% reduction to about a 40% reduction, about a 15% reduction to about a 35% reduction, about a 15% reduction to about a 30% reduction, about a 15% reduction to about a 25% reduction, about a 15% reduction to about a 20% reduction, about a 20% reduction to about a 99% reduction, about a 20% reduction to about a 90% reduction, about a 20% reduction to about a 80% reduction, about a 20% reduction to about a 70% reduction, about a 20% reduction to about a 60% reduction, about a 20% reduction to about a 50% reduction, about a 20% reduction to about a 45% reduction, about a 20% reduction to about a 40% reduction, about a 20% reduction to about a 35% reduction, about a 20% reduction to about a 30% reduction, about a 20% reduction to about a 25% reduction, about a 25% reduction to about a 99% reduction, about a 25% reduction to about a 90% reduction, about a 25% reduction to about a 80% reduction, about a 25% reduction to about a 70% reduction, about a 25% reduction to about a 60% reduction, about a 25% reduction to about a 50% reduction, about a 25% reduction to about a 45% reduction, about a 25% reduction to about a 40% reduction, about a 25% reduction to about a 35% reduction, about a 25% reduction to about a 30% reduction, about a 30% reduction to about a 99% reduction, about a 30% reduction to about a 90% reduction, about a 30% reduction to about a 80% reduction, about a 30% reduction to about a 70% reduction, about a 30% reduction to about a 60% reduction, about a 30% reduction to about a 50% reduction, about a 30% reduction to about a 45% reduction, about a 30% reduction to about a 40% reduction, about a 30% reduction to about a 35% reduction, about a 35% reduction to about a 99% reduction, about a 35% reduction to about a 90% reduction, about a 35% reduction to about a 80% reduction, about a 35% reduction to about a 70% reduction, about a 35% reduction to about a 60% reduction, about a 35% reduction to about a 50% reduction, about a 35% reduction to about a 45% reduction, about a 35% reduction to about a 40% reduction, about a 40% reduction to about a 99% reduction, about a 40% reduction to about a 90% reduction, about a 40% reduction to about a 80% reduction, about a 40% reduction to about a 70% reduction, about a 40% reduction to about a 60% reduction, about a 40% reduction to about a 50% reduction, about a 40% reduction to about a 45% reduction, about a 45% reduction to about a 99% reduction, about a 45% reduction to about a 90% reduction, about a 45% reduction to about a 80% reduction, about a 45% reduction to about a 70% reduction, about a 45% reduction to about a 60% reduction, about a 45% reduction to about a 50% reduction, about a 50% reduction to about a 99% reduction, about a 50% reduction to about a 90% reduction, about a 50% reduction to about a 80% reduction, about a 50% reduction to about a 70% reduction, about a 50% reduction to about a 60% reduction, about a 60% reduction to about a 99% reduction, about a 60% reduction to about a 90% reduction, about a 60% reduction to about a 80% reduction, about a 60% reduction to about a 70% reduction, about a 70% reduction to about a 99% reduction, about a 70% reduction to about a 90% reduction, about a 70% reduction to about a 80% reduction, about a 80% reduction to about a 99% reduction, about a 80% reduction to about a 90% reduction, or about a 90% reduction to about a 99% reduction. In some embodiments, after deep measured depletion of B cells in a tissue of a subject (e.g., in peripheral blood), B cell counts may increase and substantially recover to normal levels, e.g., as compared to levels in the subject prior to treatment or the levels in a similar subject not treated or receiving a different treatment or any other suitable control. In some embodiments, B cell counts will substantially recover to normal levels after about 3, 4, 5, 6, 7, 8, 9, 10, 11, or 12 months. In some embodiment, recovered B cells will show a sustained naive phenotype profile, indicating the potential for an immune reset and a long-term functional cure of the subject. In some embodiments, the amount of anti-GAD65 antibodies produced by the recovered B cells and measured in serum is reduced by at least 20%, 30%, 40%, 50%, 60%, 70%, 80%, 90%, 95%, or 99% relative to the amount of anti-GAD65 antibodies prior to the provided treatment. In some embodiments, the amount of anti-GlyR antibodies produced by the recovered B cells and measured in serum is reduced by at least 20%, 30%, 40%, 50%, 60%, 70%, 80%, 90%, 95%, or 99% relative to the amount of anti-GlyR antibodies prior to the provided treatment. In some embodiments, the amount of anti-amphiphysin antibodies produced by the recovered B cells and measured in serum is reduced by at least 20%, 30%, 40%, 50%, 60%, 70%, 80%, 90%, 95%, or 99% relative to the amount of anti-amphiphysin antibodies prior to the provided treatment. In some embodiments, the amount of anti-GABARAP antibodies produced by the recovered B cells and measured in serum is reduced by at least 20%, 30%, 40%, 50%, 60%, 70%, 80%, 90%, 95%, or 99% relative to the amount of anti-GABARAP antibodies prior to the provided treatment. Without wishing to be bound by theory, it is contemplated that the B cell depletion caused by the CAR T cells may lead to an immune reset, e.g., as evidenced by the durable absence of symptoms of the disease even in the presence of reconstituted B cell numbers. In some embodiments, administration of methods and compositions described herein result in a reduction (e.g., at least a 1% reduction, at least a 5% reduction, at least a 10% reduction, at least a 15% reduction, at least a 20% reduction, at least a 25% reduction, at least a 30% reduction, at least a 35% reduction, at least a 40% reduction, at least a 45% reduction, at least a 50% reduction, at least a 55% reduction, at least a 60% reduction, at least a 65% reduction, at least a 70% reduction, at least a 75% reduction, at least a 80% reduction, at least a 85% reduction, at least a 90% reduction, at least a 95% reduction, or at least a 99% reduction, or about a 1% reduction to about a 99% reduction (or any of the subranges of this range described herein) in the level of auto-antibodies in the subject having SPS, e.g., as compared to the levels in the subject prior to treatment or the levels in a similar subject not treated or receiving a different treatment.

Technologies of the present disclosure will be further described in the following examples, which do not limit the scope of the disclosure described in the claims.

### EXAMPLES

The following examples are put forth to provide those of ordinary skill in the art with a description of how the compositions and methods described herein may be used, made, and evaluated, and are intended to be purely exemplary of the disclosure and are not intended to limit the scope of what the inventors regard as their invention.

### Example 1. Preparation of Engineered T Cells Comprising a Lentivirus Vector Encoding Hu19-CD828Z

This example describes a method of producing a fully human autologous anti-CD19 chimeric antigen receptor (CAR) T cell therapy called KYV-101.

A Hu19-CD828Z may be prepared as described in U.S. Pat. No. 10,287,350. Briefly, fully human anti-CD19 CARs were generated by utilizing sequences of the fully human 47G4 monoclonal antibody (described in U.S. Patent Application Publication No. 2010/0104509). The 47G4 antibody was generated by vaccinating mice of the KM strain, which carry a human kappa light chain transgene and a human heavy chain transchromosome. The sequences of the 47G4 antibody light chain and heavy chain variable regions were obtained from U.S. Patent Application Publication No. 2010/0104509. A 47G4 scFv was designed comprising the following elements from 5′ to 3′: a CD8 signal sequence, the 47G4 antibody light chain variable region, a linker sequence (encoding a peptide comprising the amino acid sequence GSTSGSGKPGSGEGSTKG (SEQ ID NO: 9)) (see Cooper et al., Blood, 101(4): 1637-1644 (2003)), and the 47G4 antibody heavy chain variable region. A DNA sequence encoding a CAR was then designed comprising the following components from 5′ to 3′: the 47G4 scFv described above, part of the extracellular region and all of the transmembrane region of the human CD8 molecule, and the cytoplasmic (or intracellular) portions of the human CD28 molecule and the human CD3 zeta. molecule. This CAR was designated 47G4-CD828Z (SEQ ID NO: 13), and the sequence was synthesized by Invitrogen (Carlsbad, Calif.).

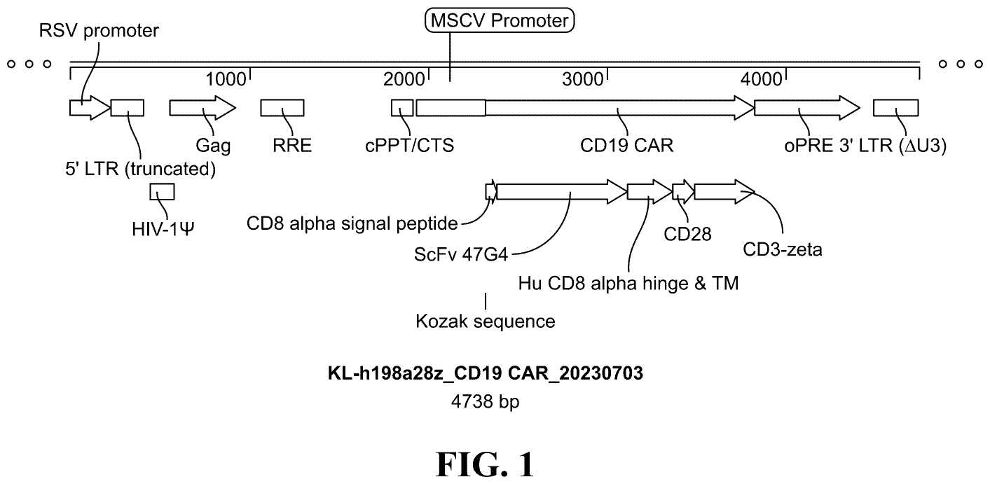

Expression of a Hu19-CD828Z polypeptide in a cell can be performed using any suitable method. In these experiments, T cells (CD4+ T cells, CD8+ T cells, or an cell mixture enriched for CD4+ and CD8+ T cells) are transduced with a lentivirus where the lentiviral vector includes a nucleic acid sequence encoding Hu19-CD828Z polypeptide. The lentiviral vector includes an MSCV promoter among other regulatory factors (see, e.g., FIG. 1). Lentivirus is produced in HEK293 cells according to standard protocols. A KL-h198a28z lentiviral vector system may be used to transduce T cells. KL-h198a28z is a self-inactivating (SIN) vesicular stomatitis virus (VSV)-G pseudotyped 3rd generation lentiviral vector expressing human anti-CD19 chimeric antigen receptor (CAR). The lentiviral vector KL-h198a28z is manufactured using a HEK 293T cell line transiently transfected with a state-of-the-art four-plasmid system. The envelope protein encoding plasmid (pLTG1292) expresses a heterologous spike protein, the VSV-G protein, under control of the cytomegalovirus (CMV) promoter. The VSV-G envelope protein provides broad cell tropism for transduction of a wide variety of mammalian cell types. KL-h198a28z encodes the CAR construct Hu19-CD828Z, and can be used to manufacture CAR T cells for treating patients with B cell-associated diseases.

Engineered T cells can be produced from blood cells using various known methods, e.g., as described in Ghassemi et al., (2018) Cancer Immunol. Res. 6(9) and Mackensen et al., (2022) Nat. Med. 28:2124-32. In one example, white blood cells are collected from a patient by apheresis. The cells are enriched for CD4+ and CD8+ T cells and are then activated with CD3 and CD28 agonistic agents, e.g., TransAct™, a polymeric nanomatrix coated. Following activation, the cells are transduced with lentiviral vector KL-h198a28z vector encoding Hu19-CD828Z and expanded in culture. The cells are then harvested and assessed for viability, Hu19-CD828Z expression, T cell phenotype, and potency (e.g., cytotoxicity and cytokine release).

In one example of preparing engineered T cells, autologous CD4+ and CD8+ T cells were enriched from MG patients. KYV-101 was generated by transducing T cells with a lentiviral vector encoding the Hu19-CD828Z polypeptide. Expansion and functional potency of CAR-T cells from a clinical cohort of KYV-101 treated patients were assessed. It was observed that KYV-101 manufactured from MG (n=6) patients displayed CD19-specific functional activity. The CAR-T cells expanded by 15.5-66.5 fold on Day 8, prior to harvest, indicating successful expansion of the cells for therapeutic use.

### Example 2. Treatment of Refractory Myasthenia Gravis With Autologous Anti-CD19 CAR T Cell Therapy

The present example describes the treatment of a patient with severe, treatment-refractory, anti-acetylcholine receptor (anti-AchR) positive generalized myasthenia gravis using KYV-101. Additional information may be found in Haghikia, A., et al. Anti-CD19 CAR T cells for refractory myasthenia gravis. Lancet Neurol. 2023 December; 22(12):1104-1105, the contents of which are hereby incorporated by reference in their entirety.

As discussed herein, myasthenia gravis is caused, at least in part, by a B-cell-driven dysfunction of neuromuscular transmission, mediated by certain auto-antibodies, e.g., anti-AchR antibodies. The disorder clinically manifests as muscle weakness and fatigue, and poses substantial challenges in terms of effective therapy.

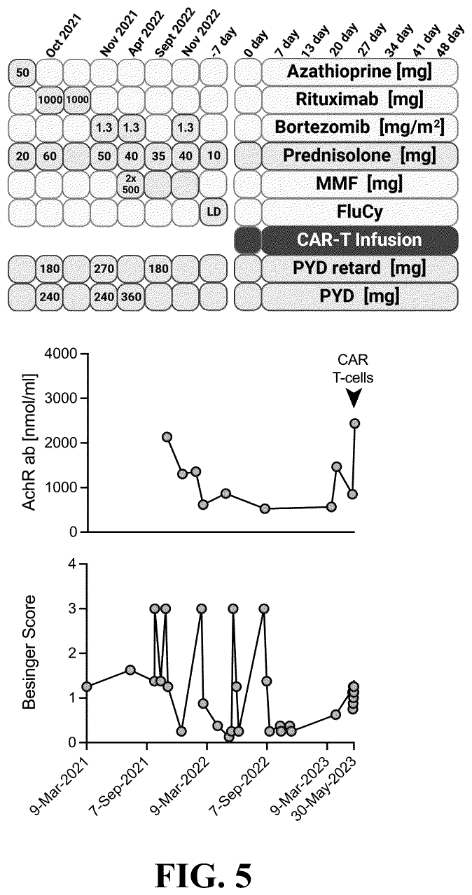

In this study, the subject treated with anti-CD19 CAR T cell therapy was a 33-year-old woman that was diagnosed with anti-AchR positive generalized myasthenia gravis about 11 years ago. In the 18 months leading up to the study, the subject had difficulties in swallowing and breathing, became unable to walk without assisting devices, and had several myasthenic crises resulting in five ICU admissions ultimately requiring invasive ventilation. Prior treatment attempts, including thymectomy, acetylcholinesterase-inhibitors, and B-cell-depleting antibodies, including rituximab, did not stabilize the disease course, which was classified as MGFA class V (defined as intubation, with or without mechanical ventilation, except when employed during routine postoperative management). Moreover, a proteasome-inhibitor (bortezomib), immunosuppressive drugs (mycophenolate mofetil), and immunoglobulin therapy were proven futile in providing long-term relief (FIG. 5). Immediately prior to the initiation of the instant study, the patient showed clinical progression despite being on glucocorticoids, mycophenolate mofetil, and bortezomib.

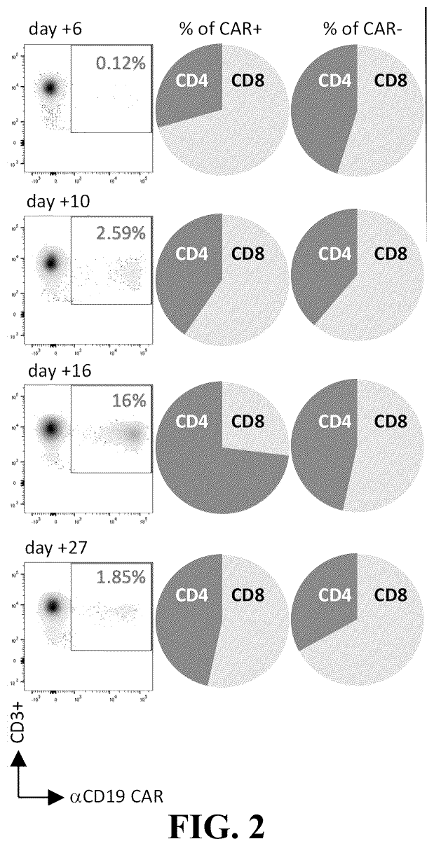

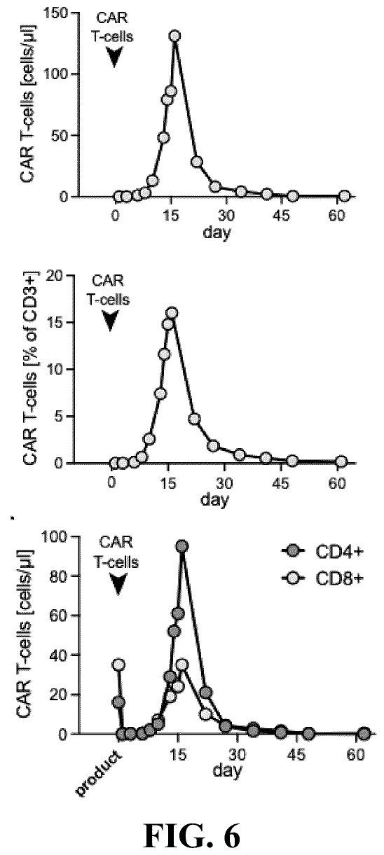

The patient was prepared for treatment with an anti-CD19 CAR T cell therapy as summarized in the following. Ongoing immunosuppression in the patient was tapered to low-dose glucocorticoids before leukapheresis and subsequent CAR T cell infusion. Mycophenolate mofetil was resumed over the manufacturing time of the autologous CAR T cell therapy, and then withdrawn 2 days before initiation of a lymphodepleting regimen. Autologous T cells obtained from the patient were transduced with a second generation anti-CD19 CAR T construct (see, e.g., Example 1, FIG. 1, and Materials and Methods section) comprising a fully human CD19 binding domain, a CD8α hinge and transmembrane domain, a CD28 co-stimulatory domain, and a CD3ζ activation domain. Following successful in vitro expansion and lymphodepletion with fludarabine (30 mg/m²; on days −6, −5, and −4) and cyclophosphamide (300 mg/m²; on days −6, −5, and −4), the patient received a single infusion of 1×10⁸ anti-CD19 CAR T cells (on day 0). CAR T cells reached their peak expansion on day 16 (131/μL; 16% of total CD3+ T cells). Notably, CD4+ cells mainly drove CAR T cell expansion. By day 62, CAR T cells were still detectable (0.5/μL; 0.19% of total CD3+ T cells) (FIGS. 2 and 6).

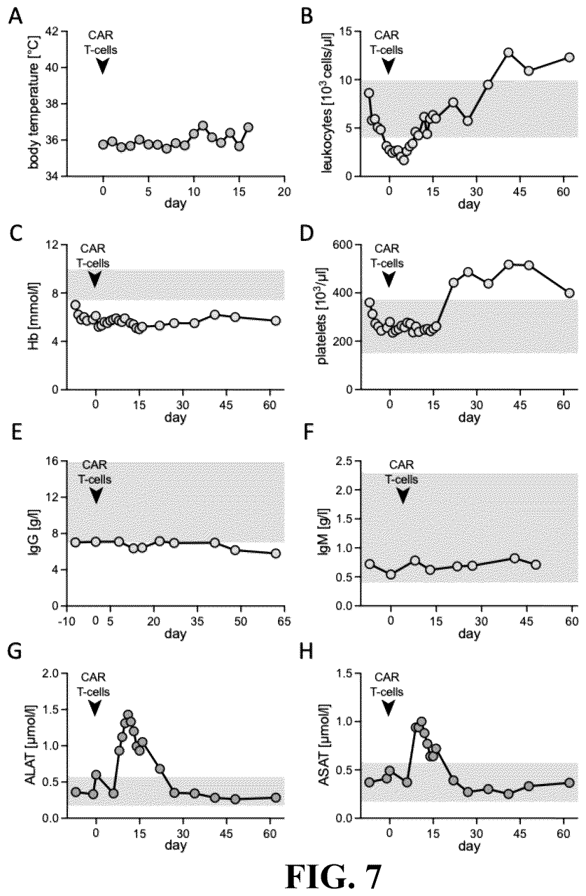

The patient had no adverse events related to CAR T cell therapy, such as cytokine release syndrome, immune effector cell-associated neurotoxicity syndrome, insufficient haematopoietic reconstitution (except a pre-existing sideropenic anaemia), nor hypogammaglobulinemia <5 g/dL. We observed a self-limiting and resolving, non-treatment-requiring grade 1 (Common Terminology for Adverse Events Version 5) transaminitis (FIG. 7).

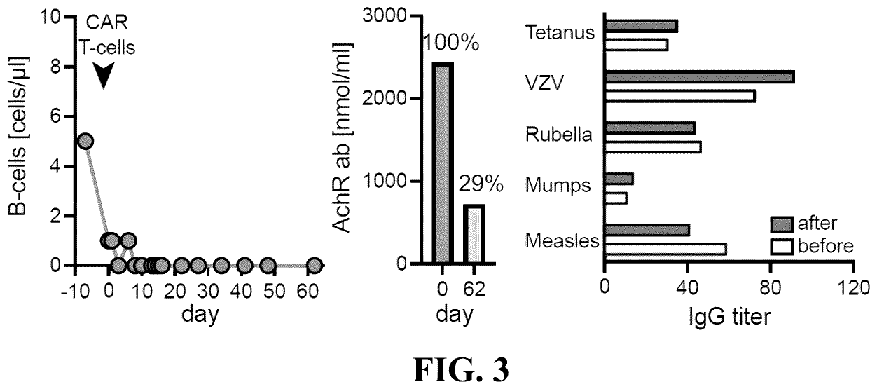

Circulating CD19+ B cells, already reduced due to prior treatments, were eliminated by day 8 and were not reconstituted at day 62 (FIG. 3). A 70% reduction in pathogenic anti-AchR antibodies from 2434 nmol/mL at day 0 to 718 nmol/mL was measured at day 62, while protective vaccination IgG titers were maintained (FIG. 3). These observations indicate that a significant fraction of pathogenic anti-AchR autoantibodies are produced by plasmablasts and short-lived plasma cells, which do express CD19 and therefore are susceptible to being depleted by KYV-101 while protective autoantibodies, produced by bone marrow long lived plasma cells that do not express CD19 are shielded from the impact of CD19 CAR T cells.

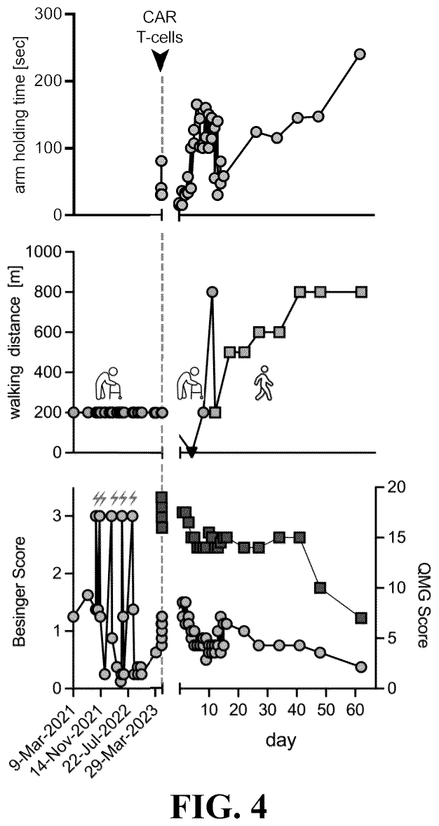

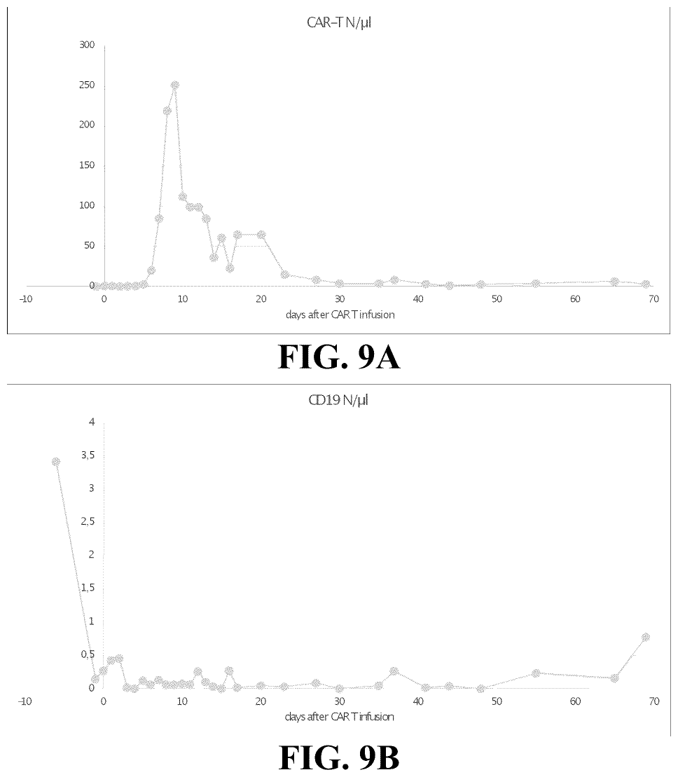

The serological findings were paralleled by the patient's improved muscle strength and fatigue over the first 2 months after CD19 CAR T infusion, evidenced by the steady increase of the arm-holding time, her enhanced walking ability without any supportive measures, and reduction of the clinical multiparameter Besinger disease activity and the quantitative myasthenia gravis scores (FIG. 4, also see FIG. 10, Patient #1, for further QMG score data points). These results were observed despite very limited exposure to prednisolone (10 mg/day) and pyridostigmine (360 mg/day of its short and 180 mg/day of its long-acting form).

The present study demonstrates, among other things, that a CAR T-cell approach that targets CD19 with a stably-expressed CAR, delivered following a conventional lymphodepleting regimen is effective in severe and refractory myasthenia gravis.

### Materials and Methods

### Anti-CD19 CAR T Cell Manufacturing

The fully human autologous anti-CD19 CAR T cell therapy, KYV-101, used in this example was composed of enriched and expanded autologous patient-derived total CD3+ T cells that were genetically modified to express a CAR that targets CD19. The fully human CAR construct contained in KYV-101 comprises a fully human anti-CD19 scFv (47G4), CD8α hinge, and transmembrane domains, a CD28 costimulatory domain, and a CD3ζ activation domain as previously described.

Following leukapheresis, manufacturing of autologous KYV-101 CAR T cells was performed. The cells were enriched for CD4+ and CD8+ cells, then activated and transduced with a third-generation lentiviral vector encoding the anti-CD19 CAR. The cells were then expanded before harvest, formulation, and cryopreservation. The final KYV-101 product was tested for identity, potency (percent CAR-positive cells and cell viability) and safety (RCL, sterility, mycoplasma, and endotoxin). After meeting acceptance criteria, the KYV-101 product was shipped back to the clinical site using a validated cryo-shipper.

The apheresis product of the patient contained 58% CD3+ T cells with 23% CD3+CD4+, 31% CD3+CD8+ and 0.6% CD3+CD4+CD8+. The cells were enriched to 83% CD3+(34% CD3+CD4+, 39% CD3+CD8+ T cells, and 1.25% CD3+CD4+CD8+) before transduction. During culture, total cells expanded 59-fold and transduction rate was 67%, yielding 2.2×109 total viable CAR+ T cells. Final product consisted of 92% CD3+ T cells: 27% CD4+, 55% CD8+, and 3% CD4+CD8+ T cells. The final dose was 0.96×10⁸ viable CAR+ T cells, which was 96% of the target dose of 1×10⁸ CAR+ cells and met the acceptance criteria.

### Previous MG Treatment

In November 2021, the patient in this example was admitted due to a myasthenic crisis. The anti-acetylcholine receptor (AChR) antibody titer was measured at above 2000 nmol/ml. Prior to this admission, the patient had undergone B-cell-depleting therapy using 2 ×1 g monoclonal anti-CD20 antibodies (i.e., rituximab). In addition, the patient was receiving prednisolone 60 mg/d orally (p.o.) and high-dose acetylcholinesterase inhibitors as part of her treatment. Due to the patient's delicate condition, experiencing severe myasthenic crises requiring intubation and ventilation, off-label treatment with the proteasome inhibitor bortezomib (1.3 mg/m2 subcutaneously [s.c.]) was initiated. Additionally, a thymectomy was performed in April 2022. However, during the first two cycles of bortezomib treatment another myasthenic crisis occurred. As a result, immunosuppression was expanded by adding mycophenolate mofetil (MMF, 1 g/d p.o.).

Prior to the present study, the patient was under treatment with bortezomib (at 4-6-month intervals), prednisolone up to 40 mg/d p.o. and MMF 1 g/d p.o., while the clinical condition worsened as underlined by an increasing Besinger score, and the anti-AChR titer 10 increased to 1465 nmol/1 (FIG. 5). One week before apheresis, the immunosuppressive therapy was reduced to a low-dose steroid (prednisolone, 10 mg/d p.o.). After apheresis, MMF (1 g/d p.o.) treatment was resumed in combination with low-dose steroid until the patient's admission for the scheduled anti-CD19 CAR T-cell infusion. From admission (d −8) through the time of collection of data for this example, the patient had been receiving 10 mg/d prednisolone as an immunosuppressive regimen.

The anti-AChR titer continued to rise within the interval between apheresis and admission and reached 2434 ng/ml after administration of lymphodepletion with cyclophosphamide and fludarabine and immediately before administering anti-CD19 CAR T cells, further highlighting the refractory nature of the disease.

### Flow Cytometry

The following anti-human antibodies were used for flow cytometry: anti-CD3 (clone SK7), anti-CD4 (clone SK3), anti-CD8 (clone RPA-T8), anti-CD45 (clone H130; all BD Biosciences, Heidelberg, Germany), CD19 CAR Detection Reagent, and Biotin antibody (clone REA746; both Miltenyi Biotec, Bergisch-Gladbach, Germany). 7AAD (BD Biosciences) was used for dead cell exclusion. Absolute cell counts were determined with BD Trucount tubes (BD Biosciences) according to manufacturer's instructions. For monitoring of CAR T-cells, patient's whole blood was stained with CD19 CAR detection reagent, washed once, and stained with a cocktail of the Biotin antibody, 7-AAD and a standardized panel of antibodies against CD45, CD3, CD4, and CD8. Data was acquired on a FACSLyric™ (BD Biosciences) and analyzed by FlowJo v10 software (BD, Franklin Lakes, NJ, USA).

In another example, a subject with concomitant myasthenia gravis and rheumatoid arthritis was treated with KYV-101. It was observed that the patient regained muscle strength, experienced no joint pain or swelling and was generally symptom-free, now capable of jogging for over 1 hour (see Haghikia, A., et al. Annals of the Rheumatic Diseases. 27 Jun. 2024. doi: 10.1136/ard-2024-226017), which is hereby incorporated by reference in its entirety.

### Example 3. Treatment of Concomitant Refractory Myasthenia Gravis and Lambert-Eaton Myasthenic Syndrome with Autologous Anti-CD19 CAR T Cell Therapy

The present example describes the treatment of a patient with severe, treatment-refractory concomitant myasthenia gravis and Lambert-Eaton myasthenic syndrome (LEMS) using a fully human autologous anti-CD19 CAR T cell therapy, KYV-101.

Myasthenia gravis (MG) and Lambert-Eaton myasthenic syndrome (LEMS) are disorders of neuromuscular transmission mediated by autoantibodies directed against functional pre- and postsynaptic components of the neuromuscular junction (NMJ). Clinically MG and LEMS are characterized by fluctuating weakness affecting ocular, faciobulbar, and extremity muscles in MG and predominantly proximal leg muscles together with additional autonomic dysfunction in LEMS. Despite therapeutic advances in recent years, some patients still are difficult to treat and experience only partial or transient responses to currently available treatments. Also, concomitant MG and LEMS pose distinctive therapeutic challenges.

Recently, CAR-T-lymphocytes directed against CD19 have been reported as a promising treatment option in systemic lupus erythematosus (SLE), another autoimmune disease mediated by pathogenic autoantibodies. This study describes the successful immunotherapy of a patient with severe MG and LEMS using autologous, fully human anti-CD19 CAR-T lymphocytes.

In this study, the subject treated with anti-CD19 CAR T cell therapy was a 33-year-old patient who was diagnosed with concomitant MG and LEMS and underwent early thymectomy. Anti-acetylcholine receptor (AChR) and anti-calcium channel autoantibodies were detected in the patient's serum. Characteristic of LEMS, the patient presented with a Trendelenburg sign during walking. The patient was receiving 100 g of intravenous immunoglobulin (IVIG) at a 2-3 week interval, required ventilatory support, and was dependent on an electric wheelchair for mobility. At the time of the present study, her condition deteriorated to MGFA class IV B status.

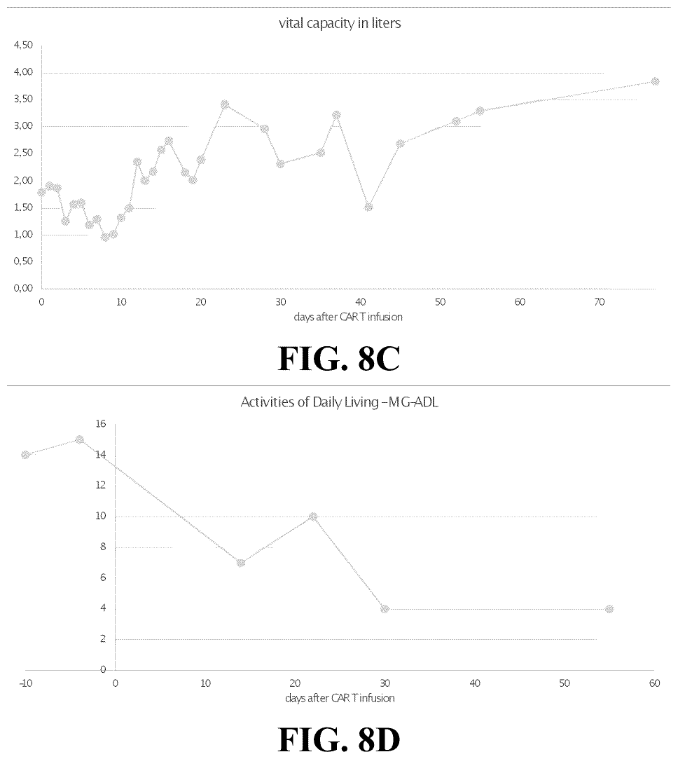

The patient was prepared for treatment with an anti-CD19 CAR T cell therapy as summarized in the following. In brief, autologous lymphocytes were collected by leukapheresis. CD3+ selected T cells were transduced with a second-generation anti-CD19 CAR-T construct (see, e.g., Example 1 and FIG. 1). The anti-CD19 CAR T cell therapy, KYV-101, incorporates a fully human CD19-binding domain, a CD8α hinge with transmembrane domain, a CD28 costimulatory domain, and a CD3ζ activation region. This construct has demonstrated lower cytokine production and reduced toxicity in comparison to preceding CARs. CAR-T cells were generated by in vitro expansion and lentiviral transduction resulting in CAR/KYV-101 expression in 74% of CD3+ T-lymphocytes. Following lymphodepletion (fludarabine, 30 mg/m2; cyclophosphamide, 300 mg/m2; days −5 to −3) and 2 days of rest, the patient was infused with 1×10⁸ anti-CD19 CAR-T lymphocytes (day 0). A measurable expansion of CAR-T cells was observed starting on day +4 after infusion. Peak expansion of CAR-T cells was observed on day +9 (251/μL; 56% of total CD3+ T cells). Notably, CAR-T cells were still detectable on day +69 (3/μL; 0.25% of CD3+; FIG. 9A). In summary, generation of functional autologous CAR-T cells was feasible, although the patient had previously received T cell-targeted immunosuppressive drugs, such as MMF and cyclophosphamide.

Following CAR-T cell administration, the patient experienced flu-like symptoms (fever, muscle and joint pain) and mild arterial hypotension (no vasopressors required), consistent with a cytokine release syndrome (CRS) maximum grade 2. Minor neurological side effects were mild fatigue, dysarthria, and dysgraphia, in summary interpreted as immune effector cell associated neurological syndrome (ICANS) grade 1. Importantly, all side effects were successfully treated with tocilizumab, dexamethasone, and anakinra. At day +10 all symptoms of CRS and ICANS had resolved completely. On day +16 an emerging local maxillary inflammation due to a previously unrecognized pulpitis of a tooth was immediately treated by a dental specialist. Subsequently, the patient was discharged after sustained clinical recovery including early gain of muscle strength.

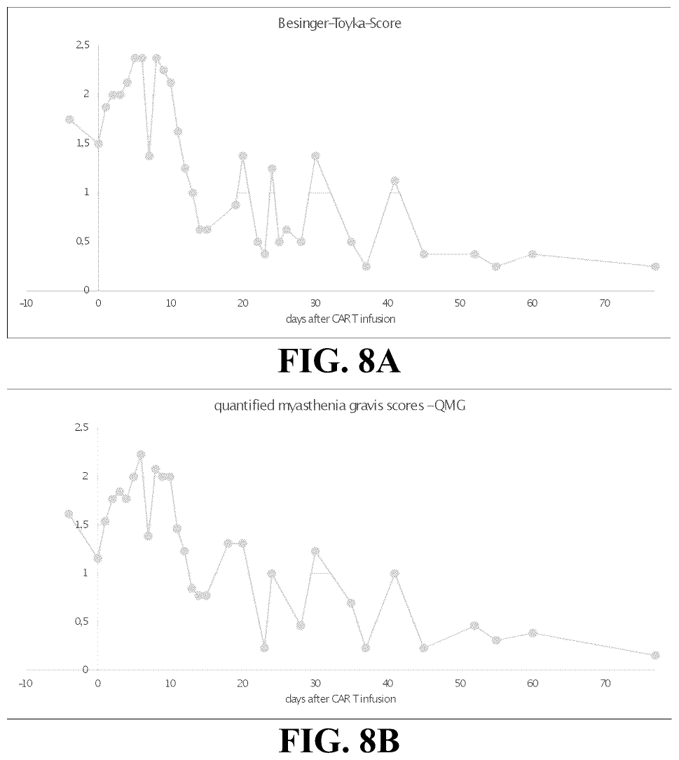

Within the next weeks, the patient achieved full mobility with complete regression of the Trendelenburg sign by day +60. The Besinger-Toyka score decreased from a maximum of 2.38 (19/8 points; before CAR-T) to 0.25 (2/8 points) on day +77 (FIG. 8A), and the QMG score fell from 2.23 (29/13 points; before CAR-T) to 0.15 (2/13 points; FIG. 8B, and also see, FIG. 10, Patient #2, for further QMG score data points). Of note, the QMG scores in FIG. 8B are shown on a 39/13 scale. They can be converted to the 0-39 scale (as in FIG. 4, the bottom panel, and FIG. 10) by multiplying the numbers by 13. In parallel, significant improvements in quality of life and activities of daily living were noticed as assessed by MG-ADL scorings (FIG. 8D). Finally, the patient was able to walk 5 km without assistance, and she resumed e-bicycling with routes up to 26 km. Her pulmonary vital capacity improved to 3.91 (98% of age-adjusted normal; FIG. 8C) at day +77. Also, the strength of her lid and small ocular muscles continued to show major improvements. This overall restitution of muscle functions was observed irrespective of continuous tapering of prednisolone to 5 mg/d and IVIG to 10-20 g every 2-3 weeks.

Consistent with the mechanism of action and anticipated depletion of a broad range of CD19-expressing B cell subpopulations, circulating CD19+ B cells were effectively depleted (FIG. 9B) as well as decreasing levels of the pathogenic AChR (A-53%) and VGCC (A-94%) autoantibodies were detected in parallel to clinical improvements (FIGS. 9C-9D). Of note, the IgG vaccine titers for tetanus, VZV, rubella, and measles remained high and at protective levels (data not shown).

Here, we present evidence that this approach of resetting autoimmunity by broad and profound B cell depletion is effective in severe autoantibody mediated neuromuscular diseases. Anti-CD19 KYV-101/CAR-T lymphocytes were safe and induced a deep clinical and serological remission in a patient severely affected by combined MG and LEMS. Without wishing to be bound by theory, the possibility to achieve immune reset by CD19-targeted CAR-T cells in autoantibody associated disease, e.g., MG and LEMS, may be enabled by the broad and deep depletion of autoreactive B lymphocytes and plasmablasts. Although effective in B cell lymphoma and a subgroup of patients with neuromuscular disease including MG and LEMS, the CD20-directed antibody rituximab does not eliminate B cells in lymphatic tissues. Accordingly, resetting of autoimmunity cannot be expected after rituximab, even in those patients achieving a profound remission.

Successful treatment of another patient diagnosed with concomitant myasthenia gravis and LEMS has also been reported (see Motte, J., et al. Treatment of concomitant myasthenia gravis and Lambert-Eaton myasthenic syndrome with autologous CD19-targeted CAR T cells. Neuron. 2024 Jun. 5; 112(11):1757-1763.e2; doi: 10.1016/j.neuron.2024.04.014), the contents of which are hereby incorporated by reference in their entirety. In summary, we present the case of a patient with concomitant MG and LEMS, safely and successfully treated with autologous anti-CD19 CAR-T cells.

### Example 4. Use of KYV-101 to Treat Myasthenia Gravis

This example describes a phase 2 clinical study to assess the safety, tolerability, and clinical activity of KYV-101 (a fully human anti-CD19 CAR T-cell therapy) in adult subjects with refractory generalized myasthenia gravis (MG).

MG is a chronic autoimmune disease mediated by autoantibodies affecting the neuromuscular junction and characterized by muscle weakness. The symptoms fluctuate, worsen with activity and improve with rest, and progress over time, typically worsening and becoming more persistent. There are at least two clinical forms of MG. In ocular MG, weakness is limited to the eyelids and extraocular muscles whereas in generalized MG, weakness involves a combination of ocular, bulbar, limb, and respiratory muscles. The clinical manifestations of MG can vary from mild focal weakness in some patients to severe paresis, bulbar weakness and respiratory failure in others. Symptom severity can vary substantially in an individual over the course of a day and over time. Acute exacerbations of myasthenic symptoms can occur spontaneously or be precipitated by infection, surgery, pregnancy, childbirth, and certain medications and when severe, can lead to life-threatening neuromuscular respiratory failure known as myasthenic crisis needing intensive care treatment.

MG is a rare disease with an incidence of approximately 7-23 cases per million and prevalence of approximately 70-320 per million. The prevalence of the disease has been increasing since mid-20^{th} century. There is a bimodal distribution with an early peak in the second and third decades of life with a female predominance, and a late peak in the sixth to eighth decade with a male predominance.

Patients with MG are often treated symptomatically with acetylcholinesterase inhibitors to increase the amount of acetylcholine available at the neuromuscular junction. Immunosuppressants, including glucocorticoids and non-steroidal immunosuppressants, are used to target the underlying immune dysregulation. In some cases, intravenous immune globulin (IVIg) and therapeutic plasma exchange (PLEX) can have rapid effects (over days) but with short term benefit (e.g., weeks) which can be used for acute exacerbations or in transitioning to slower-acting immunosuppressants such as azathioprine or mycophenolate mofetil (MMF). Thymectomy is considered for patients with a thymoma (approximately 10-15 percent of patients) in whom a complete resection is feasible, and for patients with generalized disease without a thymoma but with AChR antibodies who are ≤60 years of age. Efgartigimod, a human IgG1 antibody Fc-fragment which binds to FcRn with a higher affinity than endogenous IgG, has been approved for generalized MG. Treatment with efgartigimod leads to reduced concentrations of all IgG subtypes and was shown to be well tolerated and efficacious in patients with generalized MG. Recently, rozanolixizumab, a humanized and chimeric monoclonal antibody was approved for generalized MG in patients who are anti-AChR or anti-MuSK antibody positive. Eculizumab, a monoclonal antibody that binds to complement protein C5 and inhibits the generation of the terminal complement complex has also been approved for use in generalized MG. Its benefit is believed to be associated with a reduction of terminal complement complex C5b-9 deposition at the neuromuscular junction. Ravalizumab and zilucoplan are also inhibitors of the complement pathway that have shown efficacy in MG. Nevertheless, at least approximately 10 percent of patients with generalized MG are refractory to, or are limited in, their treatment by toxicities associated with conventional immunosuppressive and immunomodulatory therapies, such as those described here, and new treatment options are still needed.

KYV-101 consists of autologous CD4+ and CD8+ enriched and expanded T-cells genetically engineered to express a chimeric antigen receptor that targets CD19, an antigen expressed on the surface of both normal and autoreactive B-cells in patients with autoimmune diseases. CD19-targeted CAR T-cells harness the ability of cytotoxic T-cells to directly and specifically lyse target cells, leading to effective depletion of B-cells in the circulation and in lymphoid and potentially non-lymphoid tissues. The CAR expressed by the KYV-101 cells is Hu19-CD828Z, see, e.g., Example 1.

Prior to this study, a 33-year-old female with treatment-refractory MG, was treated with KYV-101 (see, Example 2). Another patient, a 33-year-old female with coexisting Lambert-Eaton myasthenic syndrome, was also treated with KYV-101 (see, Example 3). As described herein, both patients demonstrated remarkable improvements in clinical symptoms associated with MG.

The above-mentioned evidence of efficacy of CD19-targeting CAR T cells in patients with myasthenia gravis, the unmet medical need in refractory forms of myasthenia gravis, and the anticipated manageable risks of KYV-101, support proceeding with the investigation of KYV-101 in treatment refractory myasthenia gravis.

### Patient Selection

Representative Inclusion Criteria include:
≥18 years to ≤75 years of age with a diagnosis of generalized MG;
Presence of autoantibodies to AChR, MuSK, or LRP4;
Myasthenia Gravis Foundation of America (MGFA) Class III to Class IV. Patients with disease classified as MGFA Class IIB may be included if the patient requires continuous treatment with IVIG or PLEX to be maintained at MGFA Class IIB;
MG-Activities of Daily Living (MG-ADL) total score of ≥6 at screening and at predose baseline. At least one or more of the sub-components for talking, chewing, swallowing, or breathing should include a score of ≥2 on the individual subcomponent;
In the preceding 2 years failed two monoclonal antibodies with different mechanisms of action (e.g., two antibodies that bind different antigens, or bind the same antigen but one having an anti-drug antibody issue); OR, failed at least one monoclonal antibody and required chronic plasmapheresis or IVIg to control symptoms;
Failed two different monoclonal antibodies as treatment for the preceding 1 to 2 years.
On a stable dose of glucocorticoids and/or other immunotherapies for ≥1 month prior to screening. For azathioprine, being on a stable dose for ≥2 months prior to screening is required;
No change in dose of acetylcholinesterase inhibitors for ≥2 weeks prior to screening; and
No use of IVIg or plasma exchange (PLEX) within 4 weeks of pre-dose baseline

### Dosage and Administration

KYV-101 is a cell suspension formulated in a chemically defined freezing medium (50% Plasma-Lyte A™ containing 2% human serum albumin [for a final human serum albumin concentration of 1%]+50% CryoStor10 containing dimethyl sulfoxide (DMSO) to a final concentration of 5%). The finished product is filled in a freezing bag and stored at ≤−150° C. in the vapor phase of liquid nitrogen. Lymphodepletion (LD) chemotherapy consisting of cyclophosphamide (CYC) 300 mg/m² and fludarabine (FLU) 30 mg/m² is administered intravenously daily for 3 days, 5 to 7 days prior to administration of KYV-101. KYV-101 is then administered intravenously as a single infusion.

In this study, twenty subjects will be enrolled to evaluate the safety, tolerability, and efficacy of KYV-101 in MG at the dose level of 1×10⁸ CAR+ T cells. Key objectives and endpoints are detailed in Table 1.

TABLE 1
Objectives and Endpoints

| Primary Objectives | Primary Endpoints |
|---|---|
| To characterize the safety | Incidence and severity of adverse |
| and tolerability of KYV-101 | events (AEs) and laboratory |
| | abnormalities |
| To evaluate the efficacy of | MG-ADL at 24 weeks |
| KYV-101 | |
| Secondary Objectives | Secondary Endpoints |
| To further evaluate the | QMG score at 12, 24, and 52 weeks |
| efficacy of KYV-101 | MGC score at 12, 24, and 52 weeks |
| To evaluate disease-related | Change in anti-AChR, anti-MuSK, |
| antibodies | and anti-LRP4 antibodies over time |
| To characterize the | CAR-positive T-cell counts, CAR |
| pharmacokinetics (PK) and | transgene level, B-cell counts over |
| pharmacodynamics (PD) of | time, systemic cytokine |
| KYV-101 in blood | concentrations |
| To evaluate the immunogenicity | Presence of anti-KYV-101 antibodies |
| (humoral response) of KYV-101 | |
| To assess PRO after infusion | Change from baseline in: |
| of KYV-101 | MGQOL15r, MGFA-PIS, Neuro- |
| | QOL Fatigue Scale, and EQ-5D |

### Example 5. Use of KYV-101 to Treat Stiff Person Syndrome

This example describes a phase 2 clinical study to assess the safety, tolerability, and clinical activity of KYV-101 (a fully human anti-CD19 CAR T-cell therapy) in adult subjects with refractory stiff person syndrome (SPS).

SPS is a rare progressive immune-mediated disorder of the central nervous system (CNS) that is characterized by progressive rigidity and painful spasms of predominantly axial and proximal limb muscles. The condition gradually worsens over time and left untreated, it can lead to permanent disability and in some cases, mortality. Classic SPS, associated with anti-glutamic acid decarboxylase (anti-GAD) antibodies, is the most common clinical form of SPS and is present in 70 to 80% of SPS patients. SPS is estimated to occur in 1 to 2 per million people in the United States, although large, population-based epidemiologic studies are lacking.

Treatment strategies for SPS can be broadly categorized into symptomatic or immunologic, independently or in combination, depending on symptom severity. Symptomatic therapies including agents that enhance GABAergic neurotransmission (e.g., benzodiazepines, anti-spasticity agents such as baclofen, antiepileptics) are considered in the first-line. Immunotherapy with intravenous immunoglobulin (IVIG) has shown proven benefit, while evaluation of rituximab has shown benefit in a subset of patients. Plasmapheresis has shown benefit in some patients, but may have limited and transient benefits. Treatment of SPS with agents targeting GABAergic tone combined with immunotherapy can improve the neurological manifestations of SPS patients; however, prognosis is variable and spontaneous remissions are uncommon. For patients with SPS who remain refractory to conventional immunotherapies or exhibit a rapidly progressive disease, currently auto-hematopoietic stem cell transplantation (HSCT) is considered an option of last resort, considering variable responses and the potential serious complications with HSCT.

B cells contribute to systemic autoimmunity and development of disease in several ways, most notably via cytokine production, antigen presentation and complement activation (via autoantibody production). In SPS, B cell involvement is supported by the presence of antibodies against glutamic acid decarboxylase (GAD), which is widely expressed within the CNS, catalyzing the conversion of the excitatory neurotransmitter 1-glutamate to the inhibitory GABA.

CAR-T therapy such as KYV-101 may be an effective treatment for SPS, by targeting these autoreactive B cells. Using chimeric antigen receptor (CAR) T-cell technology, engineered T cells with receptors are designed to recognize and eliminate B cells, including those that produce GAD autoantibodies. This approach aims to intervene at the root of the autoimmune response, offering a precise and potentially transformative treatment for SPS. CAR-T cell therapy holds promise as a targeted and effective intervention, addressing the autoimmune component directly and potentially halting disease progression (Dalakas 2023).

Efficacy data from therapies targeting autoantibodies support the disease-driving role of B cells in SPS and suggest that a deeper, more sustained depletion of B cells may lead to meaningfully greater efficacy.

KYV-101 consists of autologous CD4+ and CD8+ enriched and expanded T-cells genetically engineered to express a chimeric antigen receptor that targets CD19, an antigen expressed on the surface of both normal and autoreactive B-cells in patients with autoimmune diseases. CD19-targeted CAR T-cells harness the ability of cytotoxic T-cells to directly and specifically lyse target cells, leading to effective depletion of B-cells in the circulation and in lymphoid and potentially non-lymphoid tissues. The CAR expressed by the KYV-101 cells is Hu19-CD828Z, see, e.g., Example 1.

### Patient Selection

Representative inclusion criteria include:
Subject must be 18 to 75 years of age (inclusive).
Subject must have diagnosed SPS per the following criteria:
   i. Rigidity of limb and axial (trunk) muscles prominent in the abdominal and thoracolumbar paraspinal areas and making bending difficult
   ii. Clinical or electrophysiological evidence (accompanied by clinical evidence) of continuous contraction of agonist and antagonist muscles
   iii. Episodic spasms precipitated by unexpected noises, tactile stimuli, or emotional upset
   iv. Absence of any other neurologic disease that could explain the stiffness and rigidity
   v. High titer serum anti-GAD65 antibodies at screening (e.g., about 20 nmole/L or greater)—OR—seropositive for anti-glycine receptor antibodies. If anti-GAD65 antibodies are lower than the high titer threshold peripherally but positive in the cerebrospinal fluid (CSF), the subject can be included. A prior documented high titer anti-GAD65 antibody level may be acceptable subject to sponsor review.
Active symptoms (e.g., items (i)-(iii) listed above) with inadequate response to at least one immunomodulatory therapy (IVIG, rituximab, or plasmapheresis).
Stiffness index ≥2.
At least 20 of the 25 enrolled subjects should be ambulatory.

### Dosage and Administration

KYV-101 is a cell suspension formulated in a chemically defined freezing medium (50% Plasma-Lyte AM containing 2% human serum albumin [for a final human serum albumin concentration of 1%]+50% CryoStor10 containing dimethyl sulfoxide (DMSO) to a final concentration of 5%). The finished product is filled in a freezing bag and stored at ≤−150° C. in the vapor phase of liquid nitrogen. Lymphodepletion (LD) chemotherapy consisting of cyclophosphamide (CYC) 300 mg/m² and fludarabine (FLU) 30 mg/m² is administered intravenously daily for 3 days, starting 5 to 7 days prior to administration of KYV-101. KYV-101 is then administered intravenously as a single infusion.

In this study, 25 subjects will be enrolled to evaluate the safety, tolerability, and efficacy of KYV-101 in SPS at the dose level of 1×10⁸ CAR+ T cells. Key objectives and endpoints are detailed in Table 2.

TABLE 2
Objectives and Endpoints

| Primary Objectives | Primary Endpoints |
|---|---|
| To evaluate the efficacy of | Change in the timed 25-foot walk (T25- |
| KYV-101 | FW) from baseline to Week 16 |
| To evaluate the safety of | Incidence and severity of adverse events |
| KYV-101 | (AEs), adverse event of special interests |
| | (AESIs), and serious adverse events |
| | (SAEs) |
| Secondary Objectives | Secondary Endpoints |
| Key secondary objective to | Change in Modified Rankin Scale from |
| evaluate efficacy of KYV- | baseline to Week 16 |
| 101 | |
| To further evaluate the | Change in the scores of the distribution- |
| efficacy of KYV-101 | of-stiffness index from baseline to |
| | Week 16 |
| | Change in Hauser Ambulation Index |
| | Change in Heightened sensitivity scale |
| | 6-minute walk test |
| | Change in anti-GAD65 antibody levels |
| | Change in anti-glycine receptor |
| | antibodies |
| | 36 item short form survey (SF-36) |
| To characterize the PK | CAR-positive T-cell counts, CAR |
| and PD of KYV-101 | transgene level, B cell counts over |
| | time, systemic cytokine concentrations |
| To evaluate the | Presence of anti-KYV-101 antibodies |
| immunogenicity (humoral) | |
| of KYV-101. | |

### Example 6. Treatment of Stiff Person Syndrome with Autologous Anti-CD19 CAR T Cell Therapy

This example describes the treatment of a patient with treatment-refractory SPS using a fully human autologous anti-CD19 CAR T cell therapy, KYV-101. This is the first case of successful use of anti-CD19 CAR T-cells in treatment-refractory SPS.

Stiff-person syndrome (SPS) is characterized by progressive rigidity and muscle spasms, which usually affect axial and limb muscles. SPS can be classified into classic SPS or variants such as focal or segmental SPS, among others. In many patients, antibodies against amphiphysin or glutamic acid decarboxylase (GAD) can be detected. Without wishing to be bound by theory, the antineuronal immunopathology including autoantibodies and cellular mechanisms specifically targeting GABAergic inhibitory pathways and synaptic signaling machinery, are believed to contribute to disease pathogenesis. SPS associated with antibodies against amphiphysin is also often accompanied by the occurrence of neoplastic disease. Standard or approved treatments for SPS include plasma exchange, intravenous immunoglobulin, anti-CD20 directed approaches, immunosuppressants, and other treatment described herein, and have met variable success.

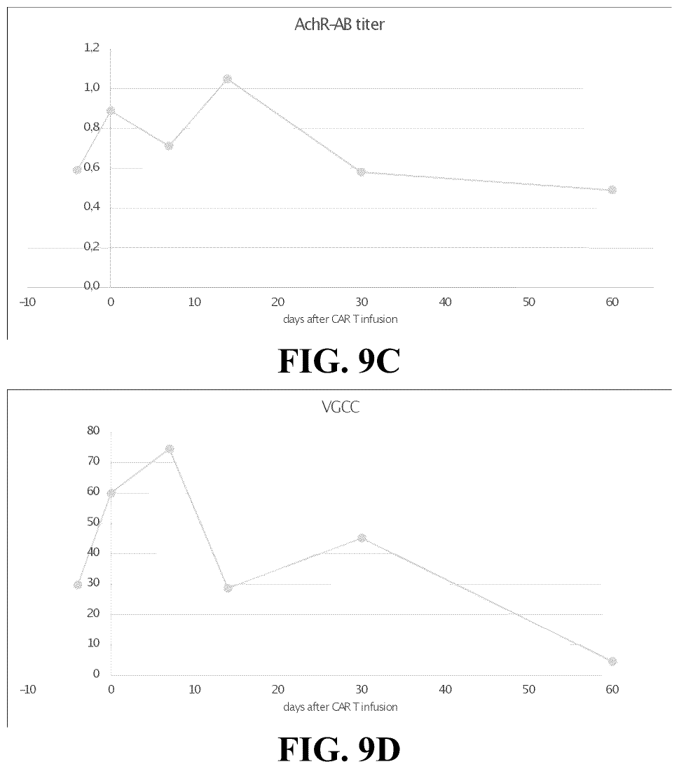

In this study, the subject treated with anti-CD19 CAR T cell therapy was a female patent that first presented with symptoms at the age of 59. The patient reported sudden loss of leg control, leading to repeated falls. Anti-GAD65 immunoglobulin (Ig)G was detected in the cerebrospinal fluid (CSF) (titer 1:10) and serum (titer 1:320). The patient was negative for onconeuronal antibodies, including anti-glycine receptor antibodies, and malignancy diagnosis including PET-CT. Treatment with corticosteroids and intravenous Ig (IVIg) resulted in only minor improvement of rigidity, and therefore a series of plasma exchanges was performed. Treatment with azathioprine, prednisolone, and diazepam (FIG. 11) led to disease stabilization for three years. Subsequent exacerbations were treated with a combination of plasma exchanges and immunoadsorptions. The patient's walking distance, however, decreased progressively to 500 meters and she required wheeled walker support. About seven years after presenting with symptoms, an increase of serum anti-GAD65 antibody-titers (1:3200) associated with gait imbalance that restricted walking ability to a few steps, was observed. Immunotherapy was escalated to rituximab (three courses), but severe leg stiffness remained and resulted in a bed-bound state. Therapy was eventually escalated using the proteasome inhibitor bortezomib with only transient stabilization.

B-cell targeting therapeutic strategy was escalated to use of an anti-CD19 CAR T-cell approach. Briefly, CAR T-cells were engineered using an anti-CD19 CAR construct (KYV-101) comprising a fully human CD19 binding domain, a CD8α hinge and transmembrane domain, a CD28 co-stimulatory domain, and a CD3ζ activation domain (see Example 1). Clinical evaluations were performed at regular intervals following administration of KYV-101. Anti-GAD65 titers were quantified using an immunofluorescence test of transfected cells.

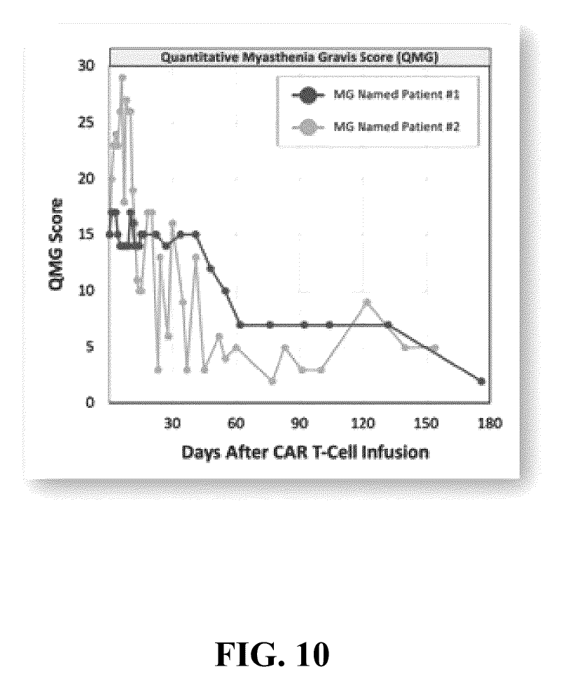

Autologous T cells were collected from the patient's peripheral blood and transduced with KYV-101. Following successful in vitro expansion and lymphodepletion with fludarabine (30 mg/m²; days −6, −5, and −4) and cyclophosphamide (300 mg/m²; days −6, −5, and −4), the patient received a single infusion of 1×10⁸ anti-CD19 CAR T cells (day 0). CAR T cells expanded beginning on day +5 to a maximum of 56.7% of all CD3+ T cells at day +14 (171 cells/μl) (FIG. 12A). B cells count remained low (FIG. 12B). Frequencies of CD8+/CD3+ T cells increased until onset of CAR T-cell expansion with subsequent decline. Thereafter, CD8+/CD3+ T cells increased again from day 42 onwards, resulting in an altered CD4/CD8 ratio (FIGS. 12C-12D).

Following CAR T-cell administration, the patient developed fever (maximum of 38.3° C.) and transient hypotension corresponding to grade 2 cytokine release syndrome (CRS) by day +9, which was successfully treated with paracetamol, dexamethasone, and tocilizumab. Indicative of tissue based expansion of anti-CD19 CAR-T cells, concurrent sore throat and cervical lymph node swelling were observed on day +9 and promptly resolved upon CRS treatment. Transient and limited (˜4-fold) increases in liver transaminases (maximum at day +15) were spontaneously reversible (day +45). No severe safety signals such as CRS grade ≥3 or immune effector-cell associated neurotoxicity syndrome grade ≥3 were observed.

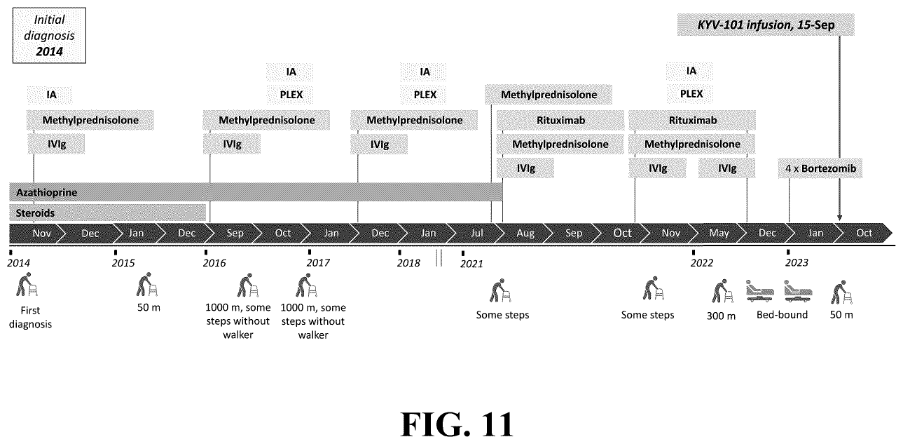

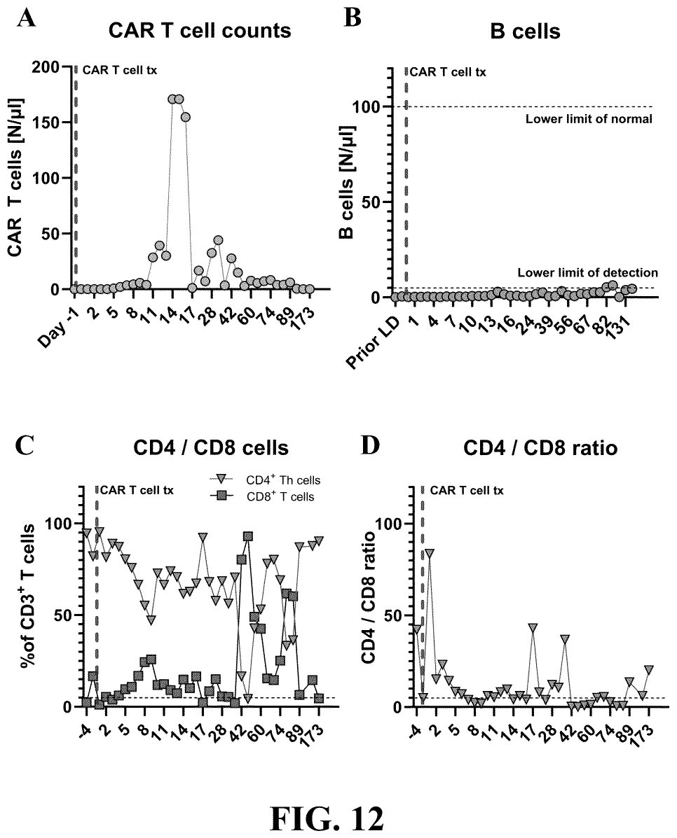

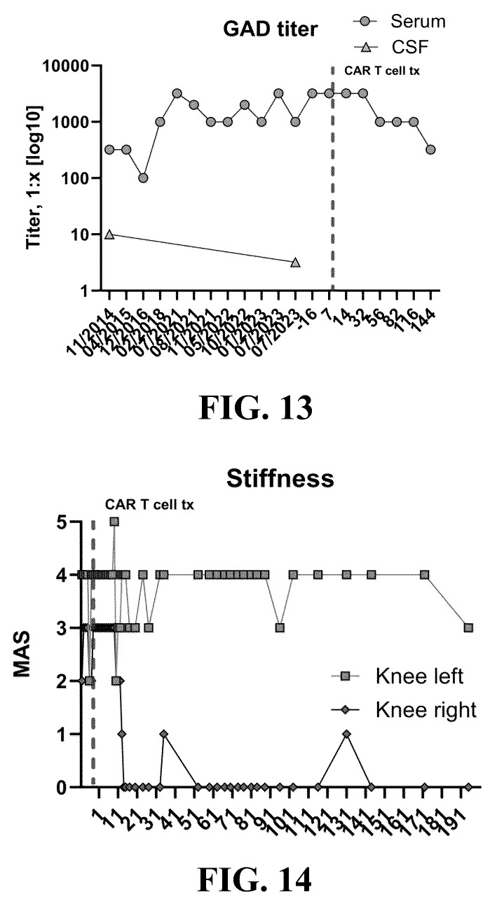

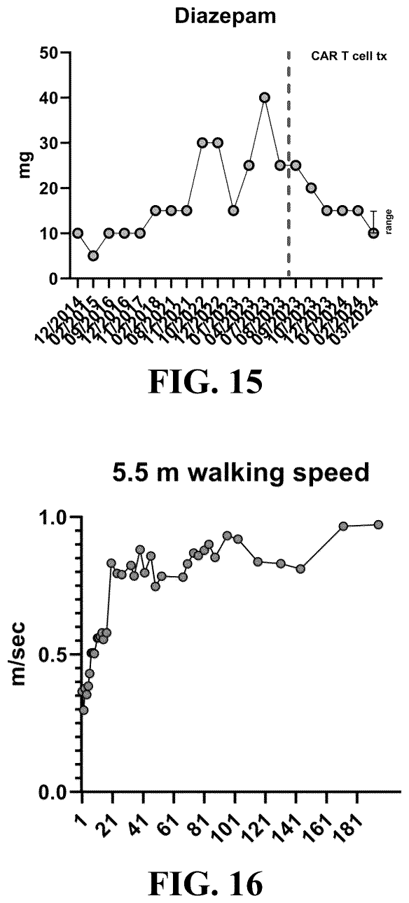

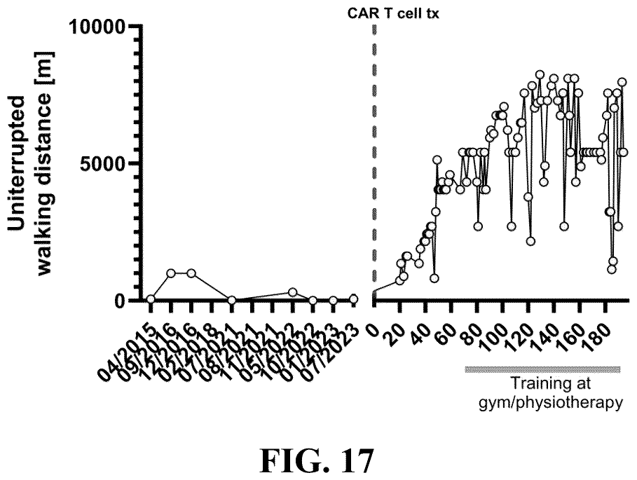

Following administration of anti-CD19 CAR T-cells, anti-GAD65 titers decreased from 1:3200 at baseline to 1:1000 at day +56, remained stable on days +82 and +116 before dropping further to 1:320 by day +144 (FIG. 13). Clinically, there was marked improvement of stiffness. Modified Ashworth scale (MAS) for the right knee decreased from 2-3 at baseline to 0 beginning at day +14. MAS for the left knee fluctuated around 4 and improved modestly over time, especially during the afternoon (FIG. 14). Pain improved from a numeric rating scale (NRS) score of 4 at baseline to several assessments of an NRS score of 0 beginning at day +50. Diazepam, a GABAergic medication given to the patient, was reduced stepwise from 25 to 10-15 mg within 5 months (FIG. 15). Fatigue, assessed using the fatigue severity scale (range 9-63), improved modestly from 48 points prior to CAR T cells to 40 points at day +112. Most impressively, walking ability improved substantially. A 5.5-meter walking test using a wheeled walker demonstrated a walking speed increase of more than 100% from approximately 0.37 m/sec at day +1 to 0.83 m/sec at day +20 (FIG. 16). Uninterrupted walking distance at home increased from several meters at baseline to more than 4 kilometers after day 50 and more than 6 kilometers after day 90 (FIG. 17). Importantly, no further immunotherapy such as IVIg was administered post CAR T-cell infusion. More details of this patient's response to the treatment can be found in Faissner, S., et al. Proc Natl Acad Sci USA. 2024 Jun. 25; 121(26): e2403227121; doi: 10.1073/pnas.2403227121, the contents of which are hereby incorporated by reference in their entirety.

In another example, a patient with SPS refractory to available treatments and co-existing MG was treated with KYV-101 at a dose of 1×10⁸ CAR T cells. KYV-101 had a favorable safety profile, with Grade 1 CRS (fever, nausea and swelling of cervical lymph nodes, on Day 7) resolved with routine standard-of-care management (acetaminophen, tocilizumab, and dexamethasone). No immune effector cell associated neurotoxicity syndrome (ICANS) has been reported in this patient. Clinical improvement of SPS was observed after KYV-101 treatment. For example, walking speed improved from 5.5 m/14.3 seconds on Day 0 to 5.5 m/6.8 seconds on Day 22; the patient's pain severity decreased, and left arm spasticity was somewhat improved. In this patient, anti-GAD65 immunoglobulin G titer decreased from 320 at baseline to 100 (Day 43) and were stable at that level subsequently (Day 65, Day 72). Similarly, anti-acetylcholine receptor autoantibodies were reduced from 0.67 nmol/L at baseline to undetectable levels on Day 36 and remained undetectable subsequently (Day 65, Day 72).

### Other Embodiments

It is to be understood that while the provided technologies have been described in conjunction with the detailed description thereof, the foregoing description is intended to illustrate and not limit the scope of the disclosure, which is defined by the scope of the appended claims. Other aspects, advantages, and modifications are within the scope of the following claims.

### SEQUENCE APPENDIX

SEQ ID NO: 1 Hu19-CD828Z Heavy Chain CDR1 (Kabat)
SYAIS
SEQ ID NO: 2 Hu19-CD828Z Heavy Chain CDR2 (Kabat)
GIIPIFGTTNYAQQFQG
SEQ ID NO: 3 Hu19-CD828Z Heavy Chain CDR3 (Kabat, Chothia)
EAVAADWLDP
SEQ ID NO: 4 Hu19-CD828Z Light Chain CDR1 (Kabat, Chothia)
RASQSVSSSYLA
SEQ ID NO: 5 Hu19-CD828Z Light Chain CDR2 (Kabat, Chothia)
GASSRAT
SEQ ID NO: 6 Hu19-CD828Z Light Chain CDR3 (Kabat, Chothia)
QQYGSSRFT
SEQ ID NO: 7 Hu19-CD828Z Heavy Chain Variable Region
QVQLVQSGAEVKKPGSSVKVSCKDSGGTFSSYAISWVRQAPGQGLEWMGGIIPIFGT
TNYAQQFQGRVTITADESTSTAYMELSSLRSEDTAVYYCAREAVAADWLDPWGQG
TLVTVSS
SEQ ID NO: 8 Hu19-CD828Z Light Chain Variable Region
EIVLTQSPGTLSLSPGERATLSCRASQSVSSSYLAWYQQKPGQAPRLLIYGASSRATGI
PDRFSGSGSGTDFTLTISRLEPEDFAVYYCQQYGSSRFTFGPGTKVDIK
SEQ ID NO: 9 Hu19-CD828Z Peptide Linker
GSTSGSGKPGSGEGSTKG
SEQ ID NO: 10 Full-length Hu19-CD828Z Chimeric Antigen Receptor without signal
peptide
EIVLTQSPGTLSLSPGERATLSCRASQSVSSSYLAWYQQKPGQAPRLLIYGASSRATGI
PDRFSGSGSGTDFTLTISRLEPEDFAVYYCQQYGSSRFTFGPGTKVDIKGSTSGSGKPG
SGEGSTKGQVQLVQSGAEVKKPGSSVKVSCKDSGGTFSSYAISWVRQAPGQGLEW
MGGIIPIFGTTNYAQQFQGRVTITADESTSTAYMELSSLRSEDTAVYYCAREAVAAD
WLDPWGQGTLVTVSSFVPVFLPAKPTTTPAPRPPTPAPTIASQPLSLRPEACRPAAGG
AVHTRGLDFACDIYIWAPLAGTCGVLLLSLVITLYCNHRNRSKRSRLLHSDYMNMTP
RRPGPTRKHYQPYAPPRDFAAYRSRVKFSRSADAPAYQQGQNQLYNELNLGRREEY
DVLDKRRGRDPEMGGKPRRKNPQEGLYNELQKDKMAEAYSEIGMKGERRRGKGH
DGLYQGLSTATKDTYDALHMQALPPR
SEQ ID NO: 11 CD8a transmembrane domain-amino acid sequence
IYIWAPLAGTCGVLLLSLVIT
SEQ ID NO: 12 CD28 transmembrane domain
FWVLVVVGGVLACYSLLVTVAFIIFWV
SEQ ID NO: 13 Full-length Hu19-CD828Z Chimeric Antigen Receptor-amino acid
sequence
MALPVTALLLPLALLLHAARPEIVLTQSPGTLSLSPGERATLSCRASQSVSSSYLAWY
QQKPGQAPRLLIYGASSRATGIPDRESGSGSGTDFTLTISRLEPEDFAVYYCQQYGSSR
FTFGPGTKVDIKGSTSGSGKPGSGEGSTKGQVQLVQSGAEVKKPGSSVKVSCKDSGG
TFSSYAISWVRQAPGQGLEWMGGIIPIFGTTNYAQQFQGRVTITADESTSTAYMELSS
LRSEDTAVYYCAREAVAADWLDPWGQGTLVTVSSFVPVFLPAKPTTTPAPRPPTPAP
TIASQPLSLRPEACRPAAGGAVHTRGLDFACDIYIWAPLAGTCGVLLLSLVITLYCNH
RNRSKRSRLLHSDYMNMTPRRPGPTRKHYQPYAPPRDFAAYRSRVKFSRSADAPAY
QQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR
SEQ ID NO: 14 Full-length Hu19-CD828Z Chimeric Antigen Receptor-nucleic acid
sequence
ATGGCCCTGCCTGTGACAGCTCTGCTGCTGCCCCTGGCCCTGCTGCTGCATGCCG
CCAGACCTGAGATCGTGCTGACCCAGTCTCCCGGCACCCTGTCTCTCAGCCCAGG
AGAGAGAGCCACCCTGAGCTGCAGAGCCAGCCAGAGCGTGTCCAGCAGCTACCT
GGCCTGGTATCAGCAGAAGCCCGGACAGGCCCCCAGACTGCTGATCTACGGCGC
CAGCTCTAGAGCCACCGGCATCCCCGACAGATTCAGCGGCAGCGGCAGTGGCAC
CGACTTCACCCTGACCATCAGCAGACTGGAACCCGAGGACTTCGCCGTGTACTAC
TGCCAGCAGTACGGCAGCAGCCGGTTCACCTTCGGCCCTGGCACCAAGGTGGAC
ATCAAGGGCAGCACCTCCGGCAGCGGCAAGCCTGGCTCTGGCGAGGGCTCTACC
AAGGGCCAGGTGCAGCTGGTGCAGTCTGGCGCCGAAGTGAAGAAACCCGGCTCT
AGCGTGAAGGTGTCCTGCAAGGACAGCGGCGGCACCTTCAGCAGCTACGCCATC
AGCTGGGTGCGCCAGGCCCCAGGACAGGGGCTGGAATGGATGGGCGGCATCATC
CCCATCTTCGGCACCACCAACTACGCCCAGCAGTTCCAGGGCAGAGTGACCATC
ACCGCCGACGAGAGCACCAGCACCGCCTACATGGAACTGAGCAGCCTGCGGAGC
GAGGACACAGCCGTGTATTACTGTGCCCGCGAGGCCGTGGCCGCCGACTGGCTG
GATCCTTGGGGACAGGGCACCCTGGTGACAGTGTCCAGCTTCGTGCCCGTGTTCC
TGCCCGCCAAGCCTACCACCACCCCTGCCCCTAGACCTCCCACCCCAGCCCCAAC
AATCGCCAGCCAGCCTCTGTCCCTGCGGCCCGAAGCCTGTAGACCTGCTGCCGGC
GGAGCCGTGCACACCAGAGGCCTGGACTTCGCCTGCGATATCTACATCTGGGCCC
CTCTGGCCGGCACCTGTGGCGTGCTGCTGCTGAGCCTGGTGATCACCCTGTACTG
CAACCACCGGAACAGAAGCAAGCGGAGCCGGCTGCTGCACAGCGACTACATGA
ACATGACCCCAAGACGGCCTGGCCCCACCCGGAAGCACTACCAGCCTTACGCCC
CTCCCAGAGACTTCGCCGCCTACCGGTCCAGAGTGAAGTTCAGCAGATCCGCCG
ACGCCCCTGCCTACCAGCAGGGACAGAACCAGCTGTACAACGAGCTGAACCTGG
GCAGACGGGAAGAGTACGACGTGCTGGACAAGCGGAGAGGCCGGGACCCCGAG
ATGGGCGGAAAGCCCAGACGGAAGAACCCCCAGGAAGGCCTGTATAACGAACT
GCAGAAAGACAAGATGGCCGAGGCCTACAGCGAGATCGGCATGAAGGGCGAGC
GGAGGCGCGGCAAGGGCCACGATGGCCTGTACCAGGGCCTGAGCACCGCCACCA
AGGACACCTACGACGCCCTGCACATGCAGGCCCTGCCCCCCAGATGA
SEQ ID NO: 15 CD8a signal peptide-amino acid sequence
MALPVTALLLPLALLLHAARP
SEQ ID NO: 16 CD8a signal peptide-nucleic acid sequence
ATGGCCCTGCCTGTGACAGCTCTGCTGCTGCCCCTGGCCCTGCTGCTGCATGCCG
CCAGACCT
SEQ ID NO: 17 Antigen binding domain with scFv 47G4-amino acid sequence
EIVLTQSPGTLSLSPGERATLSCRASQSVSSSYLAWYQQKPGQAPRLLIYGASSRATGI
PDRFSGSGSGTDFTLTISRLEPEDFAVYYCQQYGSSRFTFGPGTKVDIKGSTSGSGKPG
SGEGSTKGQVQLVQSGAEVKKPGSSVKVSCKDSGGTFSSYAISWVRQAPGQGLEW
MGGIIPIFGTTNYAQQFQGRVTITADESTSTAYMELSSLRSEDTAVYYCAREAVAAD
WLDPWGQGTLVTVSS
SEQ ID NO: 18 Antigen binding domain with scFv 47G4-nucleic acid sequence
GAGATCGTGCTGACCCAGTCTCCCGGCACCCTGTCTCTCAGCCCAGGAGAGAGA
GCCACCCTGAGCTGCAGAGCCAGCCAGAGCGTGTCCAGCAGCTACCTGGCCTGG
TATCAGCAGAAGCCCGGACAGGCCCCCAGACTGCTGATCTACGGCGCCAGCTCT
AGAGCCACCGGCATCCCCGACAGATTCAGCGGCAGCGGCAGTGGCACCGACTTC
ACCCTGACCATCAGCAGACTGGAACCCGAGGACTTCGCCGTGTACTACTGCCAG
CAGTACGGCAGCAGCCGGTTCACCTTCGGCCCTGGCACCAAGGTGGACATCAAG
GGCAGCACCTCCGGCAGCGGCAAGCCTGGCTCTGGCGAGGGCTCTACCAAGGGC
CAGGTGCAGCTGGTGCAGTCTGGCGCCGAAGTGAAGAAACCCGGCTCTAGCGTG
AAGGTGTCCTGCAAGGACAGCGGCGGCACCTTCAGCAGCTACGCCATCAGCTGG
GTGCGCCAGGCCCCAGGACAGGGGCTGGAATGGATGGGCGGCATCATCCCCATC
TTCGGCACCACCAACTACGCCCAGCAGTTCCAGGGCAGAGTGACCATCACCGCC
GACGAGAGCACCAGCACCGCCTACATGGAACTGAGCAGCCTGCGGAGCGAGGA
CACAGCCGTGTATTACTGTGCCCGCGAGGCCGTGGCCGCCGACTGGCTGGATCCT
TGGGGACAGGGCACCCTGGTGACAGTGTCCAGC
SEQ ID NO: 19 CD8a hinge and transmembrane domain-amino acid sequence
FVPVFLPAKPTTTPAPRPPTPAPTIASQPLSLRPEACRPAAGGAVHTRGLDFACDIYIW
APLAGTCGVLLLSLVITLYCNHRN
SEQ ID NO: 20 CD8a hinge and transmembrane domain-nucleic acid sequence
TTCGTGCCCGTGTTCCTGCCCGCCAAGCCTACCACCACCCCTGCCCCTAGACCTC
CCACCCCAGCCCCAACAATCGCCAGCCAGCCTCTGTCCCTGCGGCCCGAAGCCTG
TAGACCTGCTGCCGGCGGAGCCGTGCACACCAGAGGCCTGGACTTCGCCTGCGA
TATCTACATCTGGGCCCCTCTGGCCGGCACCTGTGGCGTGCTGCTGCTGAGCCTG
GTGATCACCCTGTACTGCAACCACCGGAAC
SEQ ID NO: 21 CD28 intracellular domain fragment-amino acid sequence
RSKRSRLLHSDYMNMTPRRPGPTRKHYQPYAPPRDFAAYRS
SEQ ID NO: 22 CD28 intracellular domain fragment-nucleic acid sequence
AGAAGCAAGCGGAGCCGGCTGCTGCACAGCGACTACATGAACATGACCCCAAGA
CGGCCTGGCCCCACCCGGAAGCACTACCAGCCTTACGCCCCTCCCAGAGACTTCG
CCGCCTACCGGTCC
SEQ ID NO: 23 CD3 zeta intracellular domain fragment-amino acid sequence
RVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQAL
PPR
SEQ ID NO: 24 CD3 zeta intracellular domain fragment-amino acid sequence
AGAGTGAAGTTCAGCAGATCCGCCGACGCCCCTGCCTACCAGCAGGGACAGAAC
CAGCTGTACAACGAGCTGAACCTGGGCAGACGGGAAGAGTACGACGTGCTGGAC
AAGCGGAGAGGCCGGGACCCCGAGATGGGCGGAAAGCCCAGACGGAAGAACCC
CCAGGAAGGCCTGTATAACGAACTGCAGAAAGACAAGATGGCCGAGGCCTACA
GCGAGATCGGCATGAAGGGCGAGCGGAGGCGCGGCAAGGGCCACGATGGCCTG
TACCAGGGCCTGAGCACCGCCACCAAGGACACCTACGACGCCCTGCACATGCAG
GCCCTGCCCCCCAGA
SEQ ID NO: 25 Hu19-CD828Z Heavy Chain CDR1 (Chothia)
GGTFSSY
SEQ ID NO: 26 Hu19-CD828Z Heavy Chain CDR2 (Chothia)
IPIFGT
SEQ ID NO: 27 Hinge-Transmembrane-Intracellular portion of Hu19-CD828Z Chimeric
Antigen Receptor
FVPVFLPAKPTTTPAPRPPTPAPTIASQPLSLRPEACRPAAGGAVHTRGLDFACDIYIW
APLAGTCGVLLLSLVITLYCNHRNRSKRSRLLHSDYMNMTPRRPGPTRKHYQPYAPP
RDFAAYRSRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTATKDTYD
ALHMQALPPR
SEQ ID NO: 28 CD8a hinge domain-amino acid sequence
FVPVFLPAKPTTTPAPRPPTPAPTIASQPLSLRPEACRPAAGGAVHTRGLDFACD
SEQ ID NO: 29 anti-CD19 Antibody 1 VH
EVQLVQSGAEVKKPGESLKISCKGSGYSFTNSWIGWVRQMPGKGLEWMGLIYPDDS
DTRYSPSFQGQVTISADSAINTAYLQWSSLKASDTAMYYCARQSTYIYGGYYDTWG
QGTLVTVSS
SEQ ID NO: 30 anti-CD19 Antibody 1 VL
QSVVTQPPSVSGAPGQRVTISCTGSSSNIGAGYDVHWYQQLPGTAPKLLIYENTNRPS
GVPDRFSGSKSGTSASLAITGLQAEDEADYYCQSYDSSLSGWRVFGGGTKLTVLG
SEQ ID NO: 31 anti-CD19 Antibody 2 VH
EVQLVQSGAEVKKPGESLKISCKGSGYSFTSYWIGWVRQMPGKGLEWMGIIYPGDS
DTRYSPSFQGQVTISADKSISTAYLQWSSLKASDTAMYYCARLSYSWSSWYWDFWG
QGTLVTVSS
SEQ ID NO: 32 anti-CD19 Antibody 2 VL
QAVLTQPPSVSEAPRQRVTISCSGSSSNIGNNAVSWYQQLPGKAPKLLIYYDDLLPSG
VSDRFSGSKSGTSASLAISGLQSEDEADYYCAAWDDSLNGWVFGGGTKVTVLG
SEQ ID NO: 33 anti-CD19 FMC63 VH
EVKLQESGPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAMDYWGQ
GTSVTVSS
SEQ ID NO: 34 anti-CD19 FMC63 VL
DIQMTQTTSSLSASLGDRVTISCRASQDISKYLNWYQQKPDGTVKLLIYHTSRLHSGV
PSRFSGSGSGTDYSLTISNLEQEDIATYFCQQGNTLPYTFGGGTKLEIT
SEQ ID NO: 35 anti-CD19 Antibody 1 Heavy Chain CDR1
GYSFTNSW
SEQ ID NO: 36 anti-CD19 Antibody 1 Heavy Chain CDR2
IYPDDSDT
SEQ ID NO: 37 anti-CD19 Antibody 1 Heavy Chain CDR3
ARQSTYIYGGYYDT
SEQ ID NO: 38 anti-CD19 Antibody 1 Light Chain CDR1
SSNIGAGYD
SEQ ID NO: 39 anti-CD19 Antibody 1 Light Chain CDR2
ENT
SEQ ID NO: 40 anti-CD19 Antibody 1 Light Chain CDR3
QSYDSSLSGWRV
SEQ ID NO: 41 anti-CD19 Antibody 2 Heavy Chain CDR1
GYSFTSYW
SEQ ID NO: 42 anti-CD19 Antibody 2 Heavy Chain CDR2
IYPGDSDT
SEQ ID NO: 43 anti-CD19 Antibody 2 Heavy Chain CDR3
ARLSYSWSSWYWDF
SEQ ID NO: 44 anti-CD19 Antibody 2 Light Chain CDR1
SSNIGNNA
SEQ ID NO: 45 anti-CD19 Antibody 2 Light Chain CDR2
YDD
SEQ ID NO: 46 anti-CD19 Antibody 2 Light Chain CDR3
AAWDDSLNGWV
SEQ ID NO: 47 anti-CD19 FMC63 Heavy Chain CDR1
GVSLPDY
SEQ ID NO: 48 anti-CD19 FMC63 Heavy Chain CDR2
WGSET
SEQ ID NO: 49 anti-CD19 FMC63 Heavy Chain CDR3
HYYYGGSYAMDY
SEQ ID NO: 50 anti-CD19 FMC63 Light Chain CDR1
RASQDISKYLN
SEQ ID NO: 51 anti-CD19 FMC63 Light Chain CDR2
HTSRLHS
SEQ ID NO: 52 anti-CD19 FMC63 Light Chain CDR3
QQGNTLPYT
SEQ ID NO: 53 signaling sequence
YNMN
SEQ ID NO: 54: consensus signaling sequence
YXXL/IX₍₆₋₈₎YXXL, where Y is a tyrosine residue, L/I is a leucine or isoleucine residue,
and X is any amino acid residue

What is claimed is:

1. A method of treating stiff person syndrome (SPS), the method comprising administering to a subject in need thereof a therapeutically effective amount of T cells that comprises a vector comprising a nucleic acid encoding a chimeric antigen receptor (CAR), wherein the CAR comprises, from N-terminus to C-terminus:
(a) an antigen-binding fragment of an anti-CD19 antibody;
(b) a transmembrane domain; and
(c) an intracellular T cell signaling domain from human CD3ζ.

2. The method of claim 1, wherein the subject has a high titer of anti-GAD65 antibodies in the serum.

3. The method of claim 1, wherein the subject is positive for anti-GAD65 antibodies in the cerebrospinal fluid (CSF).

4. The method of claim 1, wherein the subject is positive for anti-glycine receptor antibodies in the serum.

5. The method of any one of claims 1-4, wherein the subject has a stiffness index greater than or equal to 2.

6. The method of any one of claims 1-5, wherein the subject has:
(a) rigidity of limb and axial (trunk) muscles prominent in the abdominal and thoracolumbar paraspinal areas and making bending difficult;
(b) continuous contraction of agonist and antagonist muscles;
(c) episodic spasms precipitated by unexpected noises, tactile stimuli, or emotional upset; and
(d) no other neurologic disease that could explain the stiffness and rigidity.

7. The method of any one of claims 1-6, wherein the subject has active symptoms with inadequate response to at least one immunomodulatory therapy selected from intravenous immunoglobulin (IVIG) therapy, rituximab, or plasmapheresis.

8. The method of any one of claims 1-7, wherein the subject is ambulatory.

9. A method of treating myasthenia gravis (MG), the method comprising administering to a subject in need thereof a therapeutically effective amount of T cells that comprises a vector comprising a nucleic acid encoding a chimeric antigen receptor (CAR), wherein the CAR comprises, from N-terminus to C-terminus:
(a) an antigen-binding fragment of an anti-CD19 antibody;
(b) a transmembrane domain; and
(c) an intracellular T cell signaling domain from human CD3ζ.

10. The method of claim 9, wherein the myasthenia gravis is generalized myasthenia gravis.

11. The method of claim 9 or 10, wherein the myasthenia gravis is Class IV according to the MGFA clinical classification.

12. The method of claim 9 or 10, wherein the myasthenia gravis is Class III according to the MGFA clinical classification.

13. The method of claim 9 or 10, wherein the myasthenia gravis is Class IIB according to the MGFA clinical classification.

14. The method of claim 13, wherein the subject is maintained at Class IIB disease by continuous treatment with intravenous immunoglobulin (IVIG) therapy or plasma exchange (PLEX).

15. The method of any one of claims 9-14, wherein the subject is positive for an autoantibody that binds acetylcholine receptor (AChR), muscle-specific kinase (MuSK), or lipoprotein-related protein 4 (LRP4).

16. The method of any one of claims 9-15, wherein the subject has a MG-Activity of Daily Living (MG-ADL) total score greater than or equal to 6.

17. The method of claim 16, wherein the subject has a score greater than or equal to 2 for at least one of the sub-components in the MG-ADL score, for talking, chewing, swallowing, or breathing.

18. The method of any one of claims 9-17, wherein the subject has failed at least one monoclonal antibody treatment.

19. The method of claim 18, wherein the subject has failed, in the preceding 2 years, treatment with two or more monoclonal antibodies with different mechanisms of action, or has failed at least one monoclonal antibody and requires chronic plasmapheresis or IVIG therapy to control symptoms.

20. The method of claim 18, wherein the subject has failed two different monoclonal antibodies in the preceding 1 to 2 years.

21. The method of any one of claims 9-20, wherein the subject is on a stable dose of one or more glucocorticoids and/or other immunotherapies for at least 1 month or on a stable dose of azathioprine for at least 2 months.

22. The method of any one of claims 9-21, wherein the subject has received no change in dose of acetylcholinesterase inhibitors for at least 2 weeks.

23. The method of any one of claims 9-22, wherein the subject has not received IVIg or plasma exchange within the preceding 4 weeks.

24. The method of any one of claims 1-23, wherein the anti-CD19 antibody is a human antibody.

25. The method of any one of claims 1-24, wherein the antigen-binding fragment of the anti-CD19 antibody comprises a heavy chain variable domain and a light chain variable domain, the heavy chain variable domain comprising CDR1, CDR2, and CDR3 amino acid sequences of SEQ ID NOs: 25, 26, and 3, respectively, and the light chain variable domain comprising CDR1, CDR2, and CDR3 amino acid sequences of SEQ ID NOs: 4, 5, and 6, respectively.

26. The method of any one of claims 1-24, wherein the antigen-binding fragment of the anti-CD19 antibody comprises a heavy chain variable domain and a light chain variable domain, the heavy chain variable domain comprising CDR1, CDR2, and CDR3 amino acid sequences of SEQ ID NOs: 1, 2, and 3, respectively, and the light chain variable domain comprising CDR1, CDR2, and CDR3 amino acid sequences of SEQ ID NOs: 4, 5, and 6, respectively.

27. The method of claim 25 or 26, wherein the heavy chain variable domain comprises an amino acid sequence at least 90% identical to SEQ ID NO: 7, and the light chain variable domain comprises an amino acid sequence at least 90% identical to SEQ ID NO: 8.

28. The method of claim 27, wherein the heavy chain variable domain comprises the amino acid sequence of SEQ ID NO: 7, and the light chain variable domain comprises the amino acid sequence of SEQ ID NO: 8.

29. The method of claim 28, wherein the antigen-binding fragment of the anti-CD19 antibody comprises the amino acid sequence of SEQ ID NO: 17.

30. The method of any one of claims 1-29, wherein the transmembrane domain is from human CD8.

31. The method of any one of claims 1-30, wherein the transmembrane domain comprises an amino acid sequence at least 90% identical to SEQ ID NO: 11.

32. The method of claim 31, wherein the transmembrane domain comprises the amino acid sequence of SEQ ID NO: 11.

33. The method of any one of claims 1-32, wherein the intracellular T cell signaling domain from human CD3ζ comprises an amino acid sequence at least 90% identical to SEQ ID NO: 23.

34. The method of claim 33, wherein the intracellular T cell signaling domain from human CD3ζ comprises the amino acid sequence of SEQ ID NO: 23.

35. The method of any one of claims 1-34, wherein the CAR further comprises an intracellular T cell signaling domain from human CD28.

36. The method of claim 35, wherein the intracellular T cell signaling domain from human CD28 comprises the amino acid sequence of SEQ ID NO: 21.

37. The method of any one of claims 1-34, wherein the CAR does not comprise an intracellular T cell signaling domain from 4-1BB.

38. The method of any one of claims 1-37, wherein the CAR comprises an amino acid sequence of SEQ ID NO: 10 or 13.

39. The method of any one of claims 1-38, wherein the vector is a lentivirus vector.

40. The method of any one of claims 1-39, wherein the vector further comprises a murine stem cell virus (MSCV) U3 promoter operably linked to the nucleic acid.

41. The method of any one of claims 1-40, wherein the T cells comprise at least 10% of CD8⁺ cytotoxic T cells.

42. The method of any one of claims 1-41, wherein the T cells comprise at least 10% of CD4⁺ helper T cells.

43. The method of any one of claims 1-42, wherein the therapeutically effective dose is about 1×10⁸ of the T cells.

44. The method of any one of claims 1-43, wherein the T cells are administered by intravenous infusion.

45. The method of any one of claims 1-44, wherein the subject receives a single dose of the T cells.

46. The method of any one of claims 1-45, wherein the subject has received a lymphodepletion treatment.

47. The method of any one of claims 1-45, wherein the subject has received a minimal lymphodepletion treatment resulting in about 50% reduction of lymphocytes in the subject as compared to the amount of lymphocytes in the subject prior to the minimal lymphodepletion treatment.


SEQUENCE LISTING (54 sequences)

SEQ ID NO: 1 - protein, 5 aa, synthetic construct
SYAIS

SEQ ID NO: 2 - protein, 17 aa, synthetic construct
GIIPIFGTTNYAQQFQG

SEQ ID NO: 3 - protein, 10 aa, synthetic construct
EAVAADWLDP

SEQ ID NO: 4 - protein, 12 aa, synthetic construct
RASQSVSSSYLA

SEQ ID NO: 5 - protein, 7 aa, synthetic construct
GASSRAT

SEQ ID NO: 6 - protein, 9 aa, synthetic construct
QQYGSSRFT

SEQ ID NO: 7 - protein, 119 aa, synthetic construct
QVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSS

SEQ ID NO: 8 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIK

SEQ ID NO: 9 - protein, 18 aa, synthetic construct
GSTSGSGKPGSGEGSTKG

SEQ ID NO: 10 - protein, 481 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 21 aa, synthetic construct
IYIWAPLAGTCGVLLLSLVIT

SEQ ID NO: 12 - protein, 27 aa, synthetic construct
FWVLVVVGGVLACYSLLVTVAFIIFWV

SEQ ID NO: 13 - protein, 502 aa, synthetic construct
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - DNA, 1509 bp, synthetic construct
  source 1..1509: mol_type = other DNA
atggccctgcctgtgacagctctgctgctgcccctggccctgctgctgcatgccgccagacctgagatcgtgctgacccagtctcccggcaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggcaccgacttcaccctgaccatcagcagactggaacccgaggacttcgccgtgtactactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtgaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcttcgtgcccgtgttcctgcccgccaagcctaccaccacccctgcccctagacctcccaccccagccccaacaatcgccagccagcctctgtccctgcggcccgaagcctgtagacctgctgccggcggagccgtgcacaccagaggcctggacttcgcctgcgatatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagatga

SEQ ID NO: 15 - protein, 21 aa, synthetic construct
MALPVTALLLPLALLLHAARP

SEQ ID NO: 16 - DNA, 63 bp, synthetic construct
  source 1..63: mol_type = other DNA
atggccctgcctgtgacagctctgctgctgcccctggccctgctgctgcatgccgccagacct

SEQ ID NO: 17 - protein, 245 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSS

SEQ ID NO: 18 - DNA, 735 bp, synthetic construct
  source 1..735: mol_type = other DNA
gagatcgtgctgacccagtctcccggcaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggcaccgacttcaccctgaccatcagcagactggaacccgaggacttcgccgtgtactactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtgaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagc

SEQ ID NO: 19 - protein, 83 aa, synthetic construct
FVPVFLPAKPTTTPAPRPPTPAPTIASQPLSLRPEACRPAAGGAVHTRGLDFACDIYIWAPLAGTCGVLLLSLVITLYCNHRN

SEQ ID NO: 20 - DNA, 249 bp, synthetic construct
  source 1..249: mol_type = other DNA
ttcgtgcccgtgttcctgcccgccaagcctaccaccacccctgcccctagacctcccaccccagccccaacaatcgccagccagcctctgtccctgcggcccgaagcctgtagacctgctgccggcggagccgtgcacaccagaggcctggacttcgcctgcgatatctacatctgggcccctctggccggcacctgtggcgtgctgctgctgagcctggtgatcaccctgtactgcaaccaccggaac

SEQ ID NO: 21 - protein, 41 aa, synthetic construct
RSKRSRLLHSDYMNMTPRRPGPTRKHYQPYAPPRDFAAYRS

SEQ ID NO: 22 - DNA, 123 bp, synthetic construct
  source 1..123: mol_type = other DNA
agaagcaagcggagccggctgctgcacagcgactacatgaacatgaccccaagacggcctggccccacccggaagcactaccagccttacgcccctcccagagacttcgccgcctaccggtcc

SEQ ID NO: 23 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - DNA, 336 bp, synthetic construct
  source 1..336: mol_type = other DNA
agagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 25 - protein, 7 aa, synthetic construct
GGTFSSY

SEQ ID NO: 26 - protein, 6 aa, synthetic construct
IPIFGT

SEQ ID NO: 27 - protein, 236 aa, synthetic construct
FVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 55 aa, synthetic construct
FVPVFLPAKPTTTPAPRPPTPAPTIASQPLSLRPEACRPAAGGAVHTRGLDFACD

SEQ ID NO: 29 - protein, 121 aa, synthetic construct
EVQLVQSGAEVKKPGESLKISCKGSGYSFTNSWIGWVRQMPGKGLEWMGLIYPDDSDTRYSPSFQGQVTISADSAINTAYLQWSSLKASDTAMYYCARQSTYIYGGYYDTWGQGTLVTVSS

SEQ ID NO: 30 - protein, 113 aa, synthetic construct
QSVVTQPPSVSGAPGQRVTISCTGSSSNIGAGYDVHWYQQLPGTAPKLLIYENTNRPSGVPDRFSGSKSGTSASLAITGLQAEDEADYYCQSYDSSLSGWRVFGGGTKLTVLG

SEQ ID NO: 31 - protein, 121 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLSYSWSSWYWDFWGQGTLVTVSS

SEQ ID NO: 32 - protein, 111 aa, synthetic construct
QAVLTQPPSVSEAPRQRVTISCSGSSSNIGNNAVSWYQQLPGKAPKLLIYYDDLLPSGVSDRFSGSKSGTSASLAISGLQSEDEADYYCAAWDDSLNGWVFGGGTKVTVLG

SEQ ID NO: 33 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 34 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 35 - protein, 8 aa, synthetic construct
GYSFTNSW

SEQ ID NO: 36 - protein, 8 aa, synthetic construct
IYPDDSDT

SEQ ID NO: 37 - protein, 14 aa, synthetic construct
ARQSTYIYGGYYDT

SEQ ID NO: 38 - protein, 9 aa, synthetic construct
SSNIGAGYD

SEQ ID NO: 39 - skipped

SEQ ID NO: 40 - protein, 12 aa, synthetic construct
QSYDSSLSGWRV

SEQ ID NO: 41 - protein, 8 aa, synthetic construct
GYSFTSYW

SEQ ID NO: 42 - protein, 8 aa, synthetic construct
IYPGDSDT

SEQ ID NO: 43 - protein, 14 aa, synthetic construct
ARLSYSWSSWYWDF

SEQ ID NO: 44 - protein, 8 aa, synthetic construct
SSNIGNNA

SEQ ID NO: 45 - skipped

SEQ ID NO: 46 - protein, 11 aa, synthetic construct
AAWDDSLNGWV

SEQ ID NO: 47 - protein, 7 aa, synthetic construct
GVSLPDY

SEQ ID NO: 48 - protein, 5 aa, synthetic construct
WGSET

SEQ ID NO: 49 - protein, 12 aa, synthetic construct
HYYYGGSYAMDY

SEQ ID NO: 50 - protein, 11 aa, synthetic construct
RASQDISKYLN

SEQ ID NO: 51 - protein, 7 aa, synthetic construct
HTSRLHS

SEQ ID NO: 52 - protein, 9 aa, synthetic construct
QQGNTLPYT

SEQ ID NO: 53 - protein, 4 aa, synthetic construct
YNMN

SEQ ID NO: 54 - protein, 16 aa, synthetic construct
  VARIANT 2..3: note = X can be any amino acid
  VARIANT 5..10: note = X can be any amino acid
  VARIANT 11..12: note = X can be any amino acid or absent
  VARIANT 14..15: note = X can be any amino acid
YXXJXXXXXXXXYXXL